(19) United States
(12) Patent Application Publication
(10) Pub. No.: US 2026/0060948 A1
(43) Pub. Date: Mar. 5, 2026

(54) METHODS FOR TREATING LIVER DISEASE

(71) Applicant: CymaBay Therapeutics, Inc., Foster City, CA (US)

(72) Inventors: Susheela Y. Carroll, San Carlos, CA (US); Yun-Jung Choi, Fremont, CA (US); Daria B. Crittenden, San Francisco, CA (US); Robert L. Martin, San Ramon, CA (US); Charles A. McWherter, Oakland, CA (US); Sarah C. Proehl, San Francisco, CA (US); Alexandra V. Steinberg, Alameda, CA (US)

(21) Appl. No.: 19/286,679

(22) Filed: Jul. 31, 2025

Related U.S. Application Data
(60) Provisional application No. 63/677,952, filed on Jul. 31, 2024.
(60) Provisional application No. 63/683,179, filed on Aug. 14, 2024.
(60) Provisional application No. 63/827,704, filed on Jun. 20, 2025.

(51) Int. Cl. A61K 31/192; A61K 9/16; A61K 31/235; A61K 31/366; A61K 31/40; A61K 31/505; A61K 31/575; A61P 1/16; A61P 17/04

(52) U.S. Cl. CPC A61K 31/192; A61K 9/1611; A61K 9/1617; A61K 9/1623; A61K 9/1652; A61K 31/235; A61K 31/366; A61K 31/40; A61K 31/505; A61K 31/575; A61P 1/16; A61P 17/04

(57) ABSTRACT

The present disclosure relates generally to treating liver diseases, such as primary biliary cholangitis (PBC), comprising administering a therapeutically effective amount of seladelpar or a pharmaceutically acceptable salt thereof

### CROSS-REFERENCE TO RELATED APPLICATIONS

This application claims the benefit under 35 U.S.C. 119(e) of United States Provisional Application Nos. 63/677,952, filed Jul. 31, 2024, 63/683,179, filed Aug. 14, 2024, and 63/827,704, filed Jun. 20, 2025, each of which is hereby incorporated by reference in its entirety.

### BACKGROUND

Liver disease is generally classified as acute or chronic based upon the duration of the disease. Liver disease may be caused by infection, injury, exposure to drugs or toxic compounds, alcohol, impurities in foods, and the abnormal build-up of normal substances in the blood, an autoimmune process, a genetic defect (such as haemochromatosis), or unknown cause(s). Liver disease is a leading cause of death worldwide. In particular, it has been seen that a diet high in fat damages the liver in ways that are surprisingly similar to hepatitis.

Primary biliary cholangitis (PBC), a chronic liver disease, is an autoimmune condition of the liver marked by the slow progressive destruction of the small bile ducts of the liver, with the intralobular ducts affected early in the condition. When these ducts are damaged, bile builds up in the liver (cholestasis) and over time damages the tissue, which can lead to scarring, fibrosis, and cirrhosis. There is no cure for PBC, and liver transplantation often becomes necessary; but medication such as ursodeoxycholic acid (UDCA, ursodiol) to reduce cholestasis and improve liver function may slow the progression to allow a normal lifespan and quality of life. UDCA is approved in the United States to treat PBC, but about 40% of patients are reported to have an inadequate response to UDCA and about 5% are reported to be intolerant to UDCA treatment. However, a Cochrane Review of UDCA in PBC in 2012 found that, although UDCA showed a reduction in biomarkers of liver pathology, jaundice, and ascites, there was no evidence in the medical literature for any benefit of UDCA on mortality or liver transplantation, while its use was associated with weight gain and costs.

Therefore, it would be desirable to develop more effective and safe pharmacological treatments for PBC in subjects who are intolerant of, or who have an inadequate response to UDCA.

### SUMMARY

The present disclosure, in one embodiment, provides a method of treating primary biliary cholangitis (PBC) in a patient in need thereof, the method comprising administering a therapeutically effective amount of seladelpar or a pharmaceutically acceptable salt thereof to the patient, wherein administration of seladelpar or a pharmaceutically acceptable salt thereof results in one or more of: (a) an improvement in cholestasis; (b) an improvement in liver injury; and (c) an improvement of pruritus.

In some embodiments, the administration results in: (a) an improvement in cholestasis; (b) an improvement in liver injury; and (c) an improvement of pruritus. In some embodiments, the administration results in one or more of: decrease and/or normalization in serum alkaline phosphatase (ALP) level, decrease and/or normalization in serum total bilirubin (TB) level, decrease in serum aspartate aminotransferase (AST) level, decrease in alanine aminotransferase (ALT) level, decrease in gamma-glutamyltransferase (GGT), and decrease in pruritus Numerical Rating Scale (NRS). In some embodiments, the seladelpar or a pharmaceutically acceptable salt thereof is administered for up to 12 months. In some embodiments, a therapeutically effective amount of seladelpar lysine is administered. Said seladelpar lysine may be seladelpar lysine dihydrate. In some embodiments, the amount of seladelpar is 5 mg/day or 10 mg/day.

The present disclosure, in one embodiment, provides a method of treating primary biliary cholangitis (PBC) in a patient in need thereof, the method comprising administering seladelpar or a pharmaceutically acceptable salt thereof to the patient in an amount equivalent to 5 mg/day or 10 mg/day of seladelpar, wherein administration of seladelpar or a pharmaceutically acceptable salt thereof results in one or more of: (a) an improvement in cholestasis; (b) an improvement in liver injury; and (c) an improvement of pruritus.

In some embodiments, the administration results in: (a) an improvement in cholestasis; (b) an improvement in liver injury; and (c) an improvement of pruritus. In some embodiments, the administration results in one or more of: decrease and/or normalization in serum alkaline phosphatase (ALP) level, decrease and/or normalization in serum total bilirubin (TB) level, decrease in serum aspartate aminotransferase (AST) level, decrease in alanine aminotransferase (ALT) level, decrease in gamma-glutamyltransferase (GGT), and decrease in pruritus Numerical Rating Scale (NRS). In some embodiments, the seladelpar or a pharmaceutically acceptable salt thereof is administered for up to 12 months. In some embodiments, 14.1 mg/day of seladelpar lysine dihydrate is administered.

The present disclosure, in one embodiment, provides a method of treating a liver disease in a patient in need thereof, comprising administering a therapeutically effective amount of seladelpar or a pharmaceutically acceptable salt thereof to the patient, wherein the patient does not have decompensated cirrhosis.

In some embodiments, the liver disease is primary biliary cholangitis (PBC). In some embodiments, seladelpar or a pharmaceutically acceptable salt thereof is administered orally. In some embodiments, the patient is intolerant of, or has an inadequate response to ursodeoxycholic acid (UDCA). In some embodiments, seladelpar or a pharmaceutically acceptable salt thereof is administered as a monotherapy. In some embodiments, the method further comprises administering a therapeutically effective amount of ursodeoxycholic acid (UDCA). Ursodeoxycholic acid (UDCA) may be administered in an amount of 13-15 mg/kg/day. In some embodiments, a therapeutically effective amount of seladelpar lysine is administered. Said seladelpar lysine may be seladelpar lysine dihydrate. In some embodiments, the amount of seladelpar is 5 mg/day or 10 mg/day. In some embodiments, the method further comprises monitoring liver function or hepatic impairment of the patient for decompensated cirrhosis.

The present disclosure, in one embodiment, provides a method of administering seladelpar therapy in a subject in need thereof, comprising administration of a therapeutically effective amount of seladelpar or a pharmaceutically acceptable salt thereof to a patient, wherein the patient does not have decompensated cirrhosis.

In some embodiments, seladelpar or a pharmaceutically acceptable salt thereof is seladelpar lysine. Said seladelpar lysine may be seladelpar lysine dihydrate. In some embodiments, the amount of seladelpar is 5 mg/day or 10 mg/day. In some embodiments, the method further comprises monitoring liver function or hepatic impairment of the patient for decompensated cirrhosis.

The present disclosure, in one embodiment, provides a method of treating a liver disease in a patient in need thereof, comprising administering a therapeutically effective amount of seladelpar or a pharmaceutically acceptable salt thereof to the patient and monitoring or testing the patient for signs of drug-induced liver injury or abnormalities.

In some embodiments, the liver disease is primary biliary cholangitis (PBC). In some embodiments, seladelpar or a pharmaceutically acceptable salt thereof is administered orally. In some embodiments, the patient is intolerant of, or has an inadequate response to ursodeoxycholic acid (UDCA). In some embodiments, seladelpar or a pharmaceutically acceptable salt thereof is administered as a monotherapy. In some embodiments, the method further comprises administering a therapeutically effective amount of ursodeoxycholic acid (UDCA). Ursodeoxycholic acid (UDCA) may be administered in an amount of 13-15 mg/kg/day. In some embodiments, a therapeutically effective amount of seladelpar lysine is administered. Said seladelpar lysine may be seladelpar lysine dihydrate. In some embodiments, the amount of seladelpar is 5 mg/day or 10 mg/day. In some embodiments, the method comprises monitoring one or more of ALT, AST, total bilirubin, ALP, jaundice, right upper quadrant pain, and eosinophilia.

The present disclosure, in one embodiment, provides a method of administering seladelpar therapy in a subject in need thereof, comprising administration of a therapeutically effective amount of seladelpar or a pharmaceutically acceptable salt thereof to a patient and monitoring or testing the patient for signs of drug-induced liver injury or abnormalities.

In some embodiments, the amount of seladelpar is 5 mg/day or 10 mg/day. In some embodiments, the method comprises monitoring one or more of ALT, AST, total bilirubin, ALP, jaundice, right upper quadrant pain, and eosinophilia.

The present disclosure, in one embodiment, provides a method of treating a liver disease in a patient in need thereof, comprising administering a therapeutically effective amount of seladelpar or a pharmaceutically acceptable salt thereof to the patient and monitoring and optionally treating the patient for bone density and/or bone health.

In some embodiments, the liver disease is primary biliary cholangitis (PBC). In some embodiments, seladelpar or a pharmaceutically acceptable salt thereof is administered orally. In some embodiments, the patient is intolerant of, or has an inadequate response to ursodeoxycholic acid (UDCA). In some embodiments, seladelpar or a pharmaceutically acceptable salt thereof is administered as a monotherapy. In some embodiments, the method further comprises administering a therapeutically effective amount of ursodeoxycholic acid (UDCA). Ursodeoxycholic acid (UDCA) may be administered in an amount of 13-15 mg/kg/day. In some embodiments, a therapeutically effective amount of seladelpar lysine is administered. Said seladelpar lysine may be seladelpar lysine dihydrate. In some embodiments, the amount of seladelpar is 5 mg/day or 10 mg/day. In some embodiments, the method further comprises stopping administration of seladelpar or a pharmaceutically acceptable salt thereof when a bone fracture or negative bone health is detected in the patient.

The present disclosure, in one embodiment, provides a method of providing seladelpar therapy to a patient in need thereof, comprising administration of a therapeutically effective amount of seladelpar, further comprising testing and optionally treating the patient for bone density and/or bone health.

In some embodiments, a therapeutically effective amount of seladelpar lysine is administered. Said seladelpar lysine may be seladelpar lysine dihydrate. In some embodiments, the amount of seladelpar is 5 mg/day or 10 mg/day. In some embodiments, the method further comprises stopping administration of seladelpar or a pharmaceutically acceptable salt thereof when a bone fracture or negative bone health is detected in the patient.

The present disclosure, in one embodiment, provides a method of treating a liver disease in a patient in need thereof, comprising administering a therapeutically effective amount of seladelpar or a pharmaceutically acceptable salt thereof to the patient, wherein the patient does not have complete biliary obstruction.

In some embodiments, the liver disease is primary biliary cholangitis (PBC). In some embodiments, seladelpar or a pharmaceutically acceptable salt thereof is administered orally. In some embodiments, the patient is intolerant of, or has an inadequate response to ursodeoxycholic acid (UDCA). In some embodiments, seladelpar or a pharmaceutically acceptable salt thereof is administered as a monotherapy. In some embodiments, the method further comprises administering a therapeutically effective amount of ursodeoxycholic acid (UDCA). Ursodeoxycholic acid (UDCA) may be administered in an amount of 13-15 mg/kg/day. In some embodiments, a therapeutically effective amount of seladelpar lysine is administered. Said seladelpar lysine may be seladelpar lysine dihydrate. In some embodiments, the amount of seladelpar is 5 mg/day or 10 mg/day. In some embodiments, the method further comprises testing the patient for biliary obstruction. In some embodiments, the method further comprises stopping the administration of seladelpar or a pharmaceutically acceptable salt thereof if biliary obstruction is suspected.

The present disclosure, in one embodiment, provides a method of providing seladelpar therapy to a patient in need thereof, comprising administering a therapeutically effective amount of seladelpar or a salt thereof, wherein the patient is not identified to have complete biliary obstruction.

In some embodiments, a therapeutically effective amount of seladelpar lysine is administered. Said seladelpar lysine may be seladelpar lysine dihydrate. In some embodiments, the amount of seladelpar is 5 mg/day or 10 mg/day. In some embodiments, the method further comprises testing the patient for biliary obstruction. In some embodiments, the method further comprises stopping the administration of seladelpar or a pharmaceutically acceptable salt thereof if biliary obstruction is suspected.

The present disclosure, in one embodiment, provides a method of treating a liver disease in a patient in need thereof, wherein the patient has renal impairment, the method comprising administering a therapeutically effective amount of seladelpar or a pharmaceutically acceptable salt thereof to the patient, wherein the amount of seladelpar is 5 mg/day or 10 mg/day.

In some embodiments, the liver disease is primary biliary cholangitis (PBC). In some embodiments, seladelpar or a pharmaceutically acceptable salt thereof is administered orally. In some embodiments, the patient is intolerant of, or has an inadequate response to ursodeoxycholic acid (UDCA). In some embodiments, seladelpar or a pharmaceutically acceptable salt thereof is administered as a monotherapy. In some embodiments, the method further comprises administering a therapeutically effective amount of ursodeoxycholic acid (UDCA). Ursodeoxycholic acid (UDCA) may be administered in an amount of 13-15 mg/kg/day. In some embodiments, a therapeutically effective amount of seladelpar lysine is administered. Said seladelpar lysine may be seladelpar lysine dihydrate. In some embodiments, the renal impairment is mild, moderate, or severe renal impairment.

The present disclosure, in one embodiment, provides a method of administering seladelpar therapy in a patient in need thereof, wherein the patient has renal impairment, the method comprising administering a therapeutically effective amount of seladelpar or a pharmaceutically acceptable salt thereof, wherein the amount of seladelpar is 5 mg/day or 10 mg/day.

In some embodiments, a therapeutically effective amount of seladelpar lysine is administered. Said seladelpar lysine may be seladelpar lysine dihydrate. In some embodiments, the renal impairment is mild, moderate, or severe renal impairment.

The present disclosure, in one embodiment, provides a method of treating a liver disease in a patient in need thereof, comprising administering a therapeutically effective amount of seladelpar or a pharmaceutically acceptable salt thereof to the patient, wherein the patient is induced emesis or performing gastric lavage if the patient is overdosed.

The present disclosure, in one embodiment, provides a method of treating patient on seladelpar therapy for overdose of seladelpar, comprising inducing emesis or performing gastric lavage.

The present disclosure, in one embodiment, provides a pharmaceutical composition comprising: seladelpar or a pharmaceutically acceptable salt thereof in an amount equivalent to 5 mg/day or 10 mg of seladelpar, butylated hydroxytoluene, colloidal silicon dioxide, croscarmellose sodium, magnesium stearate, mannitol, and microcrystalline cellulose.

In some embodiments, the pharmaceutical composition is substantially free of peroxides.

In some embodiments, the pharmaceutical composition comprises granules.

In some embodiments, the pharmaceutical composition comprises 14.1 mg of seladelpar lysine dihydrate.

The present disclosure, in one embodiment, provides a pharmaceutical dosage form comprising the pharmaceutical composition in preceding paragraphs in a capsule.

The present disclosure, in one embodiment, provides a method of increasing serum level of one or more metabolites in a patient in need thereof, the method comprising administering a therapeutically effective amount of seladelpar or a pharmaceutically acceptable salt thereof to the patient, wherein said one or more metabolites are selected from the group consisting of carnitine, acetylcarnitine, acylcarnitines, N,N,N-trimethyl-5-aminovalerate, and branched-chain amino acid catabolites.

The present disclosure, in one embodiment, provides a method of decreasing serum level of one or more metabolites in a patient in need thereof, the method comprising administering a therapeutically effective amount of seladelpar or a pharmaceutically acceptable salt thereof to the patient, wherein said one or more metabolites are selected from dicarboxylates, saturated and unsaturated fatty acids, and lipid-derived inflammatory mediators.

The present disclosure, in one embodiment, provides a method of increasing expression level of one or more genes selected from OCTN2, CPT1A, CPT2, CACT, and CRAT, comprising administering a therapeutically effective amount of seladelpar or a pharmaceutically acceptable salt thereof.

The present disclosure, in one embodiment, provides a method of determining activities of seladelpar in a subject, comprising measuring the serum level of one or more of metabolites selected from the group consisting of: carnitine, acetylcarnitine, other acylcarnitines (i.e., short-, medium- and long-chain acylcarnitines), N,N,N-trimethyl-5-aminovalerate (TMAVA), branched-chain amino acid catabolites, dicarboxylates, saturated and unsaturated fatty acids, and lipid-derived inflammatory mediators.

The present disclosure, in one embodiment, provides a method of determining activities of seladelpar in a subject, comprising measuring the serum level of the expression level of one or more genes selected from OCTN2, CPT1A, CPT2, CACT, and CRAT in the subject.

The present disclosure, in one embodiment, provides a method of treating primary biliary cholangitis (PBC) in a patient in need thereof, comprising administering a therapeutically effective amount of seladelpar or a pharmaceutically acceptable salt thereof to a subject, and measuring (a) the serum level of one or more of metabolites selected from the group consisting of: carnitine, acetylcarnitine, other acylcarnitines (i.e., short-, medium- and long-chain acylcarnitines), N,N,N-trimethyl-5-aminovalerate (TMAVA), branched-chain amino acid catabolites, dicarboxylates, saturated and unsaturated fatty acids, and lipid-derived inflammatory mediators; and/or (b) the expression level of one or more genes selected from OCTN2, CPT1A, CPT2, CACT, and CRAT in the subject.

The present disclosure, in one embodiments, provides a method of decreasing serum level of at least one cholesterol level in a patient in need thereof, the method comprising administering a therapeutically effective amount of seladelpar or a pharmaceutically acceptable salt thereof to the patient, wherein said cholesterol level is total cholesterol level, low-density lipoprotein (LDL) level, or triglyceride level.

The present disclosure, in one embodiments, provides a method of treating primary biliary cholangitis (PBC) in a patient in need thereof, the method comprising administering a therapeutically effective amount of seladelpar or a pharmaceutically acceptable salt thereof and a therapeutically effective amount of one or more statins, to the patient. The present disclosure, in one embodiments, provides a method of treating primary biliary cholangitis (PBC) in a patient in need thereof, the method comprising administering a therapeutically effective amount of seladelpar or a pharmaceutically acceptable salt thereof to the patient, wherein: (a) the patient is less than 65 years old, or 65 years old or older; (b) the patient was less than 50 years old at PBC diagnosis, or 50 years old or older; (c) the patient is white, Asian, black, Latino, or non-Latino; (d) the patient is male or female; (e) the patient is from North America, Europe, or rest-of-world; (f) the patient has serum ALP level of <350 U/L or 350 U/L at baseline; (g) the patient has Total Bilirubin (TB) level of <0.6×ULN or 0.6×ULN at baseline; (h) the patient has Total Bilirubin (TB) level of <1×ULN or 1×ULN at baseline; (i) the patient has pruritus NRS of <4 or 4 at baseline; or (j) the patient has cirrhosis or does not have cirrhosis.

### BRIEF DESCRIPTION OF THE DRAWINGS

FIG. 1 illustrates a graph showing mean levels of alkaline phosphatase (ALP) of subjects over 12 months.

FIG. 2 illustrates graphs showing changes in dicarboxylates, carnitine, butyryl carnitine (C4), N,N,N-trimethyl-5-aminovalerate and branched-chain amino acid catabolites levels from Day 1 to Week 12 after seladelpar treatments in individual PBC patients.

FIG. 3 illustrates a graph showing fold changes of dicarboxylate species with varying chain lengths detected by differential abundance from Day 1 to Week 12 after seladelpar treatment.

FIG. 4 illustrates a graph showing carnitine and acylcarnitine levels at Week 12 after seladelpar treatment.

FIG. 5 illustrates graphs showing changes in carnitine, acetyl carnitine and 3-hydroxybutyryl carnitine levels in individual PBC patients.

FIG. 6 illustrates a graph showing expression of genes in human primary hepatocytes presented as fold change over vehicle.

FIG. 7 illustrates graphs showing expression level of genes in a mouse model 12 weeks after seladelpar treatment.

FIG. 8 illustrates a graph showing mean pruritus NRS score for patient treated seladelpar 10 mg or placebo over 12 months.

FIG. 9 illustrates a graph showing percentage of patients with 3 or more point decline in pruritus NRS after seladelpar treatment.

FIG. 10 illustrates a graph showing percentage of patients with 4 or more point decline in pruritus NRS after seladelpar treatment.

FIG. 11 illustrates a graph showing percentage of patients having 0 or 1 NRS after seladelpar treatment for 3 months, 6 months, 9 months, and 12 months.

FIG. 12 illustrates a graph showing number of hours spending itching for patients treated with seladelpar and placebo at baseline, after 6 months, and after 12 months.

FIG. 13 illustrates a graph showing NRS change from baseline for patient treated with seladelpar or placebo for 12 months.

FIG. 14 illustrates a graph showing % of patients with NRS of 0 or 1 after treatment with seladelpar or placebo for 3, 6, 9, or 12 months.

FIG. 15 illustrates a graph showing PBC-40 itch domain change from baseline for patient treated with seladelpar or placebo for 12 months.

FIG. 16 illustrates a graph showing PBC-40 sleep disturbance change from baseline for patient treated with seladelpar or placebo for 12 months.

FIG. 17 illustrates a graph showing PBC-40 fatigue domain change from baseline for patient treated with seladelpar or placebo for 12 months.

FIG. 18 illustrates a graph showing PBC-40 itch domain change from baseline for patient treated with seladelpar or placebo for 12 months.

FIG. 19 illustrates a graph showing PBC-40 sleep disturbance change from baseline for patient treated with seladelpar or placebo for 12 months.

FIG. 20 illustrates a graph showing PBC-40 fatigue domain change from baseline for patient treated with seladelpar or placebo for 12 months.

FIG. 21 illustrates a graph showing % of patients with NRS>0 among patients treated with seladelpar or placebo for 3, 6, 9, or 12 months.

FIG. 22 illustrates graphs showing percent change in total cholesterol level from baseline for patient treated with seladelpar or placebo for 12 months with or without statin.

FIG. 23 illustrates graphs showing percent change in LDL-C level from baseline for patient treated with seladelpar or placebo for 12 months with or without statin.

FIG. 24 illustrates graphs showing percent change in triglycerides level from baseline for patient treated with seladelpar or placebo for 12 months with or without statin.

FIG. 25 graphs showing percent change in HDL-C level from baseline for patient treated with seladelpar or placebo for 12 months with or without statin.

It will be recognized that some or all of the figures are schematic representations for purpose of illustration.

### DETAILED DESCRIPTION

### Definitions

The following description sets forth exemplary embodiments of the present technology. It should be recognized, however, that such description is not intended as a limitation on the scope of the present disclosure but is instead provided as a description of exemplary embodiments.

As used in the present specification, the following words, phrases, and symbols are generally intended to have the meanings as set forth below, except to the extent that the context in which they are used indicates otherwise.

A dash (“-”) that is not between two letters or symbols is used to indicate a point of attachment for a substituent. For example, —C(O)NH₂ is attached through the carbon atom. A dash at the front or end of a chemical group is a matter of convenience; chemical groups may be depicted with or without one or more dashes without losing their ordinary meaning. A wavy line drawn through a line in a structure indicates a point of attachment of a group. Unless chemically or structurally required, no directionality is indicated or implied by the order in which a chemical group is written or named.

Reference to “about” a value or parameter herein includes (and describes) embodiments that are directed to that value or parameter per se. In certain embodiments, the term “about” includes the indicated amount±10%. In other embodiments, the term “about” includes the indicated amount±5%. In certain other embodiments, the term “about” includes the indicated amount±1%. Also, to the term “about X” includes description of “X”. Also, the singular forms “a” and “the” include plural references unless the context clearly dictates otherwise. Thus, e.g., reference to “the compound” includes a plurality of such compounds and reference to “the assay” includes reference to one or more assays and equivalents thereof known to those skilled in the art.

The terms “optional” or “optionally” means that the subsequently described event or circumstance may or may not occur, and that the description includes instances where said event or circumstance occurs and instances in which it does not. Also, the term “optionally substituted” refers to any one or more hydrogen atoms on the designated atom or group may or may not be replaced by a moiety other than hydrogen.

The term “pharmaceutically acceptable” indicates that the indicated material does not have properties that would cause a reasonably prudent medical practitioner to avoid administration of the material to a patient, taking into consideration the disease or conditions to be treated and the respective route of administration. For example, it is commonly required that such a material be essentially sterile, e.g., for injectables. The term “pharmaceutically acceptable salt” of a given compound refers to salts that retain the biological effectiveness and properties of the given compound, and which are not biologically or otherwise undesirable. Pharmaceutically acceptable base addition salts can be prepared from inorganic and organic bases. Salts derived from inorganic bases include, by way of example only, sodium, potassium, lithium, ammonium, calcium and magnesium salts. Salts derived from organic bases include, but are not limited to, salts of primary, secondary and tertiary amines. Specific examples of suitable amines include, by way of example only, isopropyl amine, trimethyl amine, diethyl amine, tri(iso-propyl) amine, tri(n-propyl) amine, ethanolamine, diethanolamine, 2-dimethylamino ethanol, lysine, arginine, histidine, caffeine, procaine, hydrabamine, choline, betaine, ethylenediamine, glucosamine, N-alkylglucamines, theobromine, purines, piperazine, piperidine, morpholine, N-ethylpiperidine, and the like. Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like.

As used herein, the term “hydrate” means a complex formed by combination of water molecules with molecules or ions of the solute, and “monohydrate” includes one water molecule, and “dihydrate” incudes two water molecules.

As used herein, “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

As used herein, “baseline” refers to initial set of values collected from a participant, subject, or patient in a clinical trial before the treatment begins, and may refer to the status of the subject or patient before treatment.

The “Numerical Rating Scale (NRS)” is a well-defined, reliable, and sensitive scale for evaluating the intensity of pruritus in PBC. Patients rate their worst itch in the past 24 hours on a scale of 0 (no itch) to 10 (worst itch imaginable). “Severe pruritus” or “severe itch” in this application may refer to NRS score ≥7. “Moderate pruritus” or “moderate itch” in this application may refer to NRS score <7-≥4. “Near no pruritus” in this application may refer to NRS score 0 or 1. “No pruritus” or “no itch” may refer to NRS score of 0.

The “5-D Itch Scale” measures the impact of itching on people's lives. This tool assesses the duration (hours per day), degree (severity), direction (improvement or worsening), disability (4 items addressing impact of itch on sleep, leisure/social, housework/errands, and work/school), and distribution (16 potential affected body regions; each scored with “absent” or “present”) of itch over the past 2 weeks. The duration, degree, direction, and disability domains are scored on a scale of 1 to 5, with higher scores indicating greater impairment.

The “PBC-40” is a patient-derived, disease-specific quality of life questionnaire used in clinical PBC studies that assesses 6 domains: itch (including a sleep disturbance question), fatigue, cognitive, and symptoms over the last 4 weeks, along with social and emotional overall/in general.

### Seladelpar

Seladelpar (International Nonproprietary Name-INN), having the following structure, has the chemical name [4-({(2R)-2-ethoxy-3-[4-(trifluoromethyl)phenoxy]propyl}sulfanyl)-2-methylphenoxy]acetic acid.

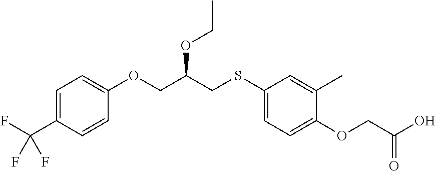 [SMILES: CCO[C@H](COc1ccc(C(F)(F)F)cc1)CSc1ccc(OCC(=O)O)c(C)c1]

Seladelpar and its synthesis, formulation, and use are disclosed in, for example, U.S. Pat. Nos. 7,301,050, 7,635,718, and 8,106,095 (compound 15 in Table 1, Example M, claim 14). Lysine (L-lysine) salts of seladelpar and related compounds are disclosed in U.S. Pat. No. 7,709,682.

Seladelpar is an orally active, potent agonist of peroxisome proliferator-activated receptor-δ (PPARδ). It is specific (>600-fold and >2500-fold compared with peroxisome proliferator-activated receptor-α and peroxisome proliferator-activated receptor-γ receptors) to PPARδ. PPARδ activation stimulates fatty acid oxidation and utilization, improves plasma lipid and lipoprotein metabolism, glucose utilization, and mitochondrial respiration, and preserves stem cell homeostasis. According to U.S. Pat. No. 7,301,050, PPARδ agonists, such as seladelpar, are suggested to treat PPARδ-mediated conditions, including “diabetes, cardiovascular diseases, Metabolic X syndrome, hypercholesterolemia, hypo-high density lipoprotein (HDL)-cholesterolemia, hyper-low density protein (LDL)-cholesterolemia, dyslipidemia, atherosclerosis, and obesity”, with dyslipidemia said to include hypertriglyceridemia and mixed hyperlipidemia.

Pharmacological activity leading to therapeutic effects includes inhibition of bile acid synthesis through activation of PPARδ, which is a nuclear receptor expressed in most tissues, including the liver. Published studies show that PPARδ activation by seladelpar reduces bile acid synthesis through Fibroblast Growth Factor 21 (FGF21)-dependent downregulation of CYP7A1, the key enzyme for the synthesis of bile acids from cholesterol, and is associated with decreases in the pruritogenic cytokine Interleukin-31 (IL-31).

Seladelpar, as a L-lysine salt dihydrate, has been studied at oral doses equivalent to 50 and 100 mg/day of seladelpar in mixed dyslipidemia; at doses equivalent to 10, 20, and 50 mg/day of seladelpar in non-alcoholic steatohepatitis; and at doses equivalent to 2, 5, 10, 50, and 200 mg/day of seladelpar in PBC.

US Application Publication No. 2019/0105291 and PCT International Publication No. WO 2019/067373 disclose the treatment of cholestatic pruritus with seladelpar and its salts.

Unless the context requires otherwise, reference to seladelpar, and reference to “a compound that is seladelpar or a salt thereof”, is a reference both to seladelpar itself and to its salts. An amount of a seladelpar salt that is “equivalent to” a particular amount of seladelpar refers to that amount of the salt that is the particular amount multiplied by the ratio of the formula weight of the salt to the formula weight of seladelpar. For example, if seladelpar L-lysine salt dihydrate is being used, since the formula weight of seladelpar L-lysine salt dihydrate is about 1.41 times the formula weight of seladelpar, an amount of about 14.1 mg/day of seladelpar L-lysine salt dihydrate will be equivalent to an amount of 5 mg/day or 10 mg/day of seladelpar.

### Pharmaceutical Compositions, Dosage Form, and Modes of Administration

Seladelpar provided herein are usually administered in the form of pharmaceutical compositions. Thus, provided herein are also pharmaceutical compositions that contain seladelpar or a pharmaceutically acceptable salt, tautomer, stereoisomer, mixture of stereoisomers, prodrug, or deuterated analog thereof and one or more pharmaceutically acceptable vehicles selected from carriers, adjuvants, and excipients. Suitable pharmaceutically acceptable vehicles may include, for example, inert solid diluents and fillers, diluents, including sterile aqueous solution and various organic solvents, permeation enhancers, solubilizers and adjuvants. Such compositions are prepared in a manner well known in the pharmaceutical art. See, e.g., Remington's Pharmaceutical Sciences, Mace Publishing Co., Philadelphia, Pa. 17th Ed. (1985); and Modem Pharmaceutics, Marcel Dekker, Inc. 3rd Ed. (G. S. Banker & C. T. Rhodes, Eds.).

The pharmaceutical compositions may be administered in either single or multiple doses. The pharmaceutical composition may be administered by various methods including, for example, rectal, buccal, intranasal, and transdermal routes. In certain embodiments, the pharmaceutical composition may be administered by intra-arterial injection, intravenously, intraperitoneally, parenterally, intramuscularly, subcutaneously, orally, topically, or as an inhalant.

One mode for administration may be oral. Administration may be via, for example, capsule or enteric coated tablets. In making the pharmaceutical compositions that include at least one compound described herein or a pharmaceutically acceptable salt, tautomer, stereoisomer, mixture of stereoisomers, prodrug, or deuterated analog thereof, the active ingredient is usually diluted by an excipient and/or enclosed within such a carrier that can be in the form of a capsule, sachet, paper, or other container. When the excipient serves as a diluent, it can be in the form of a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

In some embodiments, seladelpar may be provided as a capsule for oral administration. The pharmaceutical composition within the capsule may include about 1 mg to about 100 mg, about 1 mg to about 50 mg, about 1 mg to about 20 mg, about 1 mg to about 10 mg, about 3 mg to about 100 mg, about 3 mg to about 50 mg, about 3 mg to about 20 mg, about 3 mg to about 15 mg, about 3 mg to about 10 mg, about 5 mg to about 100 mg, about 5 mg to about 50 mg, about 5 mg to about 20 mg, about 5 mg to about 15 mg, about 5 mg to about 10 mg, about 7 mg to about 100 mg, about 7 mg to about 50 mg, about 7 mg to about 20 mg, about 7 mg to about 15 mg, about 7 mg to about 12 mg, or about 7 mg to about 10 mg equivalent amount of seladelpar. In some embodiments, the pharmaceutical composition may include about 5 mg or about 10 mg equivalent amount of seladelpar. In some embodiments, the pharmaceutical composition may include about 5 mg equivalent amount of seladelpar. In some embodiments, the pharmaceutical composition may include about 10 mg equivalent amount of seladelpar. In some embodiments, the seladelpar may be provided as lysine (e.g., L-lysine) salts dihydrate. The pharmaceutical composition may include about 7.05 mg or about 14.1 mg of seladelpar lysine dihydrate. The pharmaceutical composition may include about 7.05 mg of seladelpar lysine dihydrate. The pharmaceutical composition may include about 14.1 mg of seladelpar lysine dihydrate.

In some embodiments, the pharmaceutical composition includes seladelpar or a pharmaceutically acceptable salt, tautomer, stereoisomer, mixture of stereoisomers, prodrug, or deuterated analog thereof and one or more excipients. The pharmaceutical composition may include excipients such as fillers, binders, disintegrant, lubricant, glidant, or antioxidant. Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include lubricating agents such as talc, magnesium stearate, and mineral oil; wetting agents; emulsifying and suspending agents; preserving agents such as methyl and propylhydroxy-benzoates; sweetening agents; and flavoring agents.

The pharmaceutical composition may include one or more of the following excipients: lactose monohydrate, sodium starch glycolate, butylated hydroxyanisole, butylated hydroxytoluene, colloidal silicon dioxide, croscarmellose sodium, magnesium stearate, mannitol, and microcrystalline cellulose. The pharmaceutical composition may include two or more of the following excipients: lactose monohydrate, sodium starch glycolate, butylated hydroxyanisole, butylated hydroxytoluene, colloidal silicon dioxide, croscarmellose sodium, magnesium stearate, mannitol, and microcrystalline cellulose. The pharmaceutical composition may include three or more of the following excipients: lactose monohydrate, sodium starch glycolate, butylated hydroxyanisole, butylated hydroxytoluene, colloidal silicon dioxide, croscarmellose sodium, magnesium stearate, mannitol, and microcrystalline cellulose. The pharmaceutical composition may include four or more of the following excipients: lactose monohydrate, sodium starch glycolate, butylated hydroxyanisole, butylated hydroxytoluene, colloidal silicon dioxide, croscarmellose sodium, magnesium stearate, mannitol, and microcrystalline cellulose. The pharmaceutical composition may include five or more of the following excipients: lactose monohydrate, sodium starch glycolate, butylated hydroxyanisole, butylated hydroxytoluene, colloidal silicon dioxide, croscarmellose sodium, magnesium stearate, mannitol, and microcrystalline cellulose. In some embodiments, the pharmaceutical composition includes butylated hydroxytoluene, colloidal silicon dioxide, croscarmellose sodium, magnesium stearate, mannitol, and microcrystalline cellulose.

The pharmaceutical composition or dosage form may be free of peroxides, or substantially free of peroxides, such that oxidation of the pharmaceutical composition and seladelpar is minimized, and the pharmaceutical composition can have longer shelf life. Peroxides may be reduced or removed by inclusion of antioxidants, such as butylated hydroxytoluene or butylated hydroxyanisole in the formulation. In some embodiments, the pharmaceutical composition or dosage form includes less than about 5%, less than about 4%, less than about 3%, less than about 2%, less than about 1%, less than about 0.8%, less than about 0.6%, less than about 0.4%, less than about 0.3%, less than about 0.2%, less than about 0.1% or less than about 0.01% of any peroxides by total weight of the pharmaceutical composition or dosage form. In some embodiments, one or more of the excipients or carriers for the pharmaceutical composition is substantially free of peroxides, or completely free of peroxides. For example, one or more of colloidal silicon dioxide, croscarmellose sodium, magnesium stearate, mannitol, and microcrystalline cellulose are substantially free of peroxides. In some embodiments, one or more of colloidal silicon dioxide, croscarmellose sodium, magnesium stearate, mannitol, and microcrystalline cellulose include less than about 5%, less than about 4%, less than about 3%, less than about 2%, less than about 1%, less than about 0.8%, less than about 0.6%, less than about 0.4%, less than about 0.3%, less than about 0.2%, less than about 0.1% or less than about 0.01% of any peroxides by weight. In some embodiments, each of colloidal silicon dioxide, croscarmellose sodium, magnesium stearate, mannitol, and microcrystalline cellulose are substantially free of peroxides. In some embodiments, each of colloidal silicon dioxide, croscarmellose sodium, magnesium stearate, mannitol, and microcrystalline cellulose include less than about 5%, less than about 4%, less than about 3%, less than about 2%, less than about 1%, less than about 0.8%, less than about 0.6%, less than about 0.4%, less than about 0.3%, less than about 0.2%, less than about 0.1% or less than about 0.01% of any peroxides by weight.

In some embodiments, the dosage form or pharmaceutical composition includes seladelpar or a salt thereof in an amount of about 1% w/w to about 30% w/w, about 1% w/w to about 20% w/w, about 1% w/w to about 15% w/w, about 1% w/w to about 10% w/w, about 1% w/w to about 8% w/w about 5% w/w to about 30% w/w, about 5% w/w to about 20% w/w, about 5% w/w to about 15% w/w, about 5% w/w to about 10% w/w, about 5% w/w to about 8% w/w, or about 8% w/w to about 10% w/w. In some embodiments, the dosage form or pharmaceutical composition includes seladelpar in an amount of about 1% w/w to about 30% w/w, about 1% w/w to about 20% w/w, about 1% w/w to about 15% w/w, about 1% w/w to about 10% w/w, about 1% w/w to about 8% w/w about 5% w/w to about 30% w/w, about 5% w/w to about 20% w/w, about 5% w/w to about 15% w/w, about 5% w/w to about 10% w/w, about 5% w/w to about 8% w/w, or about 8% w/w to about 10% w/w. In some embodiments, the dosage form or pharmaceutical composition includes a salt of seladelpar (e.g., seladelpar lysine dihydrate) in an amount of about 1% w/w to about 30% w/w, about 1% w/w to about 20% w/w, about 1% w/w to about 15% w/w, about 1% w/w to about 10% w/w, about 1% w/w to about 8% w/w about 5% w/w to about 30% w/w, about 5% w/w to about 20% w/w, about 5% w/w to about 15% w/w, about 5% w/w to about 10% w/w, about 5% w/w to about 8% w/w, or about 8% w/w to about 10% w/w. In some embodiments, the dosage form or pharmaceutical composition includes a salt of seladelpar (e.g., seladelpar lysine dihydrate) in an amount of about 14.1% w/w. In some embodiments, the dosage form or pharmaceutical composition includes seladelpar (as free acid) in an amount of about 6.25% w/w. In some embodiments, the dosage form or pharmaceutical composition includes a salt of seladelpar (e.g., seladelpar lysine dihydrate) in an amount of about 8.82% w/w.

In some embodiments, the dosage form or pharmaceutical composition includes about 20% w/w to about 50% w/w, about 20% w/w to about 45% w/w, about 20% w/w to about 40% w/w, about 25% w/w to about 50% w/w, about 25% w/w to about 45% w/w, about 25% w/w to about 40% w/w, about 30% w/w to about 50% w/w, about 30% w/w to about 45% w/w, about 30% w/w to about 40% w/w, about 35% w/w to about 50% w/w, about 35% w/w to about 45% w/w, or about 35% w/w to about 40% w/w of mannitol. In some embodiments, the dosage form or pharmaceutical composition includes about 36.93% w/w of mannitol.

In some embodiments, the dosage form or pharmaceutical composition includes about 30% w/w to about 70% w/w, about 30% w/w to about 65% w/w, about 30% w/w to about 60% w/w, about 30% w/w to about 55% w/w, about 30% w/w to about 50% w/w, about 35% w/w to about 60% w/w, about 35% w/w to about 55% w/w, about 35% w/w to about 50% w/w, about 40% w/w to about 70% w/w, about 40% w/w to about 65% w/w, about 40% w/w to about 60% w/w, about 40% w/w to about 55% w/w, or about 40% w/w to about 50% w/w of microcrystalline cellulose. In some embodiments, the dosage form or pharmaceutical composition includes about 48.66% w/w of microcrystalline cellulose.

In some embodiments, the dosage form or pharmaceutical composition includes about 0.5% w/w to about 10% w/w, about 0.5% w/w to about 7.5% w/w, about 0.5% w/w to about 5% w/w, about 1% w/w to about 10% w/w, about 1% w/w to about 7.5% w/w, about 1% w/w to about 5% w/w, about 2% w/w to about 10% w/w, about 2% w/w to about 7.5% w/w, or about 2% w/w to about 5% w/w of croscarmellose sodium. In some embodiments, the dosage form or pharmaceutical composition includes about 3% w/w of croscarmellose sodium.

In some embodiments, the dosage form or pharmaceutical composition includes about 0.1% w/w to about 5% w/w, about 0.1% w/w to about 3% w/w, about 0.1% w/w to about 2% w/w, about 0.5% w/w to about 5% w/w, about 0.5% w/w to about 3% w/w, about 0.5% w/w to about 2% w/w, about 1% w/w to about 5% w/w, about 1% w/w to about 3% w/w, or about 1% w/w to about 2% w/w of magnesium stearate. In some embodiments, the dosage form or pharmaceutical composition includes about 1.5% w/w of magnesium stearate.

In some embodiments, the dosage form or pharmaceutical composition includes about 0.1% w/w to about 5% w/w, about 0.1% w/w to about 3% w/w, about 0.1% w/w to about 2% w/w, about 0.1% w/w to about 1.5% w/w, about 0.5% w/w to about 5% w/w, about 0.5% w/w to about 3% w/w, about 0.5% w/w to about 2% w/w, or about 0.5% w/w to about 1.5% w/w of colloidal silicon dioxide. In some embodiments, the dosage form or pharmaceutical composition includes about 1% w/w of colloidal silicon dioxide.

In some embodiments, the dosage form or pharmaceutical composition includes about 0.05% w/w to about 1% w/w, about 0.05% w/w to about 0.5% w/w, about 0.05% w/w to about 0.3% w/w, about 0.05% w/w to about 0.2% w/w, or about 0.05% w/w to about 0.15% w/w of butylated hydroxytoluene. In some embodiments, the dosage form or pharmaceutical composition includes about 0.05% w/w to about 1% w/w, about 0.05% w/w to about 0.5% w/w, about 0.05% w/w to about 0.3% w/w, about 0.05% w/w to about 0.2% w/w, or about 0.05% w/w to about 0.15% w/w of butylated hydroxyanisole. In some embodiments, the dosage form or pharmaceutical composition includes about 0.1% w/w of butylated hydroxytoluene. In some embodiments, the dosage form or pharmaceutical composition includes about 0.1% w/w of butylated hydroxyanisole.

In some embodiments, the dosage form or pharmaceutical composition includes about 5% w/w to about 10% w/w of seladelpar lysine dihydrate, about 30% w/w to about 40% w/w of mannitol, about 45% w/w to about 55% w/w of microcrystalline cellulose, about 1% w/w to about 5% w/w of croscarmellose sodium, about 1% w/w to about 5% w/w of magnesium stearate, about 0.5% w/w to about 3% w/w of colloidal silicon dioxide, about 0.05% w/w to about 0.5% w/w of butylated hydroxytoluene.

The pharmaceutical composition may be contained within hard gelatin capsule shells. The capsule shells may include gelatin, titanium dioxide, black iron oxide, yellow iron oxide, red iron oxide and/or colorants.

The compositions that include at least one compound described herein or a pharmaceutically acceptable salt, tautomer, stereoisomer, mixture of stereoisomers, prodrug, or deuterated analog thereof can be formulated so as to provide quick, sustained or delayed release of the active ingredient after administration to the subject by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345. Another formulation for use in the methods disclosed herein employ transdermal delivery devices (“patches”). Such transdermal patches may be used to provide continuous or discontinuous infusion of the compounds described herein in controlled amounts. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

For preparing solid compositions such as tablets or granules, the principal active ingredient may be mixed with a pharmaceutical excipient to form a solid preformulation composition containing a homogeneous mixture of a compound described herein or a pharmaceutically acceptable salt, tautomer, stereoisomer, mixture of stereoisomers, prodrug, or deuterated analog thereof. When referring to these preformulation compositions as homogeneous, the active ingredient may be dispersed evenly throughout the composition so that the composition may be readily subdivided into equally effective unit dosage forms such as tablets, pills, and capsules.

In some embodiments, seladelpar or pharmaceutically acceptable salt thereof and the excipients may be formulated as granules, which can be further contained within capsules. In some embodiments, seladelpar or pharmaceutically acceptable salt thereof and the excipients, such as butylated hydroxytoluene, colloidal silicon dioxide, croscarmellose sodium, magnesium stearate, mannitol, and microcrystalline cellulose may be formulated as granules, which can be further contained within capsules. In some embodiments, such granules may be made by roller compaction and/or dry granulation. In some embodiments, the pharmaceutical composition has intragranular portion and extragranular portion.

The tablets or pills of the compounds described herein may be coated or otherwise compounded to provide a dosage form affording the advantage of prolonged action, or to protect from the acid conditions of the stomach. For example, the tablet or pill can include an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

One mode for administration is parenteral, for example, by injection. The forms in which the pharmaceutical compositions described herein may be incorporated for administration by injection include, for example, aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Compositions for inhalation or insufflation may include solutions and suspensions in pharmaceutically acceptable, aqueous, or organic solvents, or mixtures thereof, and powders. The liquid or solid compositions may contain suitable pharmaceutically acceptable excipients as described herein. In some embodiments, the compositions are administered by the oral or nasal respiratory route for local or systemic effect. In other embodiments, compositions in pharmaceutically acceptable solvents may be nebulized by use of inert gases. Nebulized solutions may be inhaled directly from the nebulizing device or the nebulizing device may be attached to a facemask tent, or intermittent positive pressure breathing machine. Solution, suspension, or powder compositions may be administered, preferably orally or nasally, from devices that deliver the formulation in an appropriate manner.

### Method of Treating

“Treatment” or “treating” is an approach for obtaining beneficial or desired results including clinical results. Beneficial or desired clinical results may include one or more of the following: a) inhibiting the disease or condition (e.g., decreasing one or more symptoms resulting from the disease or condition, and/or diminishing the extent of the disease or condition); b) slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or c) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival.

“Prevention” or “preventing” means any treatment of a disease or condition that causes the clinical symptoms of the disease or condition not to develop. Compounds may, in some embodiments, be administered to a subject (including a human) who is at risk or has a family history of the disease or condition.

“Subject” refers to an animal, such as a mammal (including a human), that has been or will be the object of treatment, observation, or experiment. The methods described herein may be useful in human therapy and/or veterinary applications. In some embodiments, the subject is a mammal. In one embodiment, the subject is a human.

“Monitoring” or “monitor” generally refers to the overseeing, supervision, regulation, watching, tracking, or surveillance of an activity, an occurrence, a change, or a condition. For example, the term “monitoring the patient for a symptom” refers to tracking the occurrence of a symptom in a patient. Similarly, the “monitoring,” when used in connection with patient compliance, either individually, or in a clinical trial, refers to the tracking or confirming that the patient is actually taking the compound being tested as prescribed. The monitoring can be performed, for example, by following biomarkers, communicating with the subject or patient, or visual observation. Monitoring may be done in any degree or any frequency which is considered suitable by a clinician or administrator. For example, a patient may be monitored for a certain biomarker very closely and/or very frequently, for example by measuring the biomarker every week, every day, every hour, or real time. On the other hand, a patient may be monitored less often or more passively. For example, a patient may be monitored by asking the patient about occurrence of certain symptom during a monthly, or a bi-monthly follow up meeting, or by instructing the patient to contact and report certain symptom if it occurs. “Monitoring” can also include testing or checking (verbally or non-verbally) for a certain condition, symptom, or biomarker.

The term “therapeutically effective amount” or “effective amount” of a compound described herein or a pharmaceutically acceptable salt, tautomer, stereoisomer, mixture of stereoisomers, prodrug, or deuterated analog thereof means an amount sufficient to effect treatment when administered to a subject, to provide a therapeutic benefit such as amelioration of symptoms or slowing of disease progression. For example, a therapeutically effective amount may be an amount sufficient to decrease a symptom of a disease or condition of a liver disease, such as primary biliary cholangitis (PBC). The therapeutically effective amount may vary depending on the subject, and disease or condition being treated, the weight and age of the subject, the severity of the disease or condition, and the manner of administering, which can readily be determined by one or ordinary skill in the art.

The methods described herein may be applied to cell populations in vivo or ex vivo. “In vivo” means within a living individual, as within an animal or human. In this context, the methods described herein may be used therapeutically in an individual. “Ex vivo” means outside of a living individual. Examples of ex vivo cell populations include in vitro cell cultures and biological samples including fluid or tissue samples obtained from individuals. Such samples may be obtained by methods well known in the art. Exemplary biological fluid samples include blood, cerebrospinal fluid, urine, and saliva. In this context, the compounds and compositions described herein may be used for a variety of purposes, including therapeutic and experimental purposes. For example, the compounds and compositions described herein may be used ex vivo to determine the optimal schedule and/or dosing of administration of a compound of the present disclosure for a given indication, cell type, individual, and other parameters. Information gleaned from such use may be used for experimental purposes or in the clinic to set protocols for in vivo treatment. Other ex vivo uses for which the compounds and compositions described herein may be suited are described below or will become apparent to those skilled in the art. The selected compounds may be further characterized to examine the safety or tolerance dosage in human or non-human subjects. Such properties may be examined using commonly known methods to those skilled in the art.

The compounds disclosed herein are useful for the treatment of a liver disease, such as primary biliary cholangitis (PBC). In some embodiments, the liver disease may be primary sclerosing cholangitis (PSC) or nonalcoholic steatohepatitis (NASH).

In further embodiments, the methods are provided for alleviating a symptom of a liver disease, such as primary biliary cholangitis (PBC). In some embodiments, the liver disease may be primary sclerosing cholangitis (PSC) or nonalcoholic steatohepatitis (NASH).

In some embodiments, the disease or condition is a liver disease, such as primary biliary cholangitis (PBC). In some embodiments, the liver disease may be primary sclerosing cholangitis (PSC) or nonalcoholic steatohepatitis (NASH).

Measures for determining efficacy of treatment of a liver disease, such as primary biliary cholangitis (PBC), have been described and include, for example, the following: serum alkaline phosphatase (ALP) level, serum total bilirubin (TB) level, serum aspartate aminotransferase (AST) level, serum alanine aminotransferase (ALT) level, and the pruritus Numerical Rating Scale (NRS).

Seladelpar is a first-in-class, potent, and selective PPARδ agonist, or delpar, with anti-cholestatic, anti-inflammatory, and anti-pruritic activities. For example, previously known drugs, such as ursodeoxycholic acid (UDCA) or elafibranor, have not shown to alleviate pruritus, which is associated with PBC.

In some embodiments, the present disclosure provides methods of treating PBC, comprising administering a therapeutically effective amount of seladelpar or a pharmaceutically acceptable salt thereof, wherein administration of seladelpar or a pharmaceutically acceptable salt thereof results in one or more of: (a) an improvement in cholestasis; (b) an improvement in liver injury; and (c) an improvement or prevention of pruritus. In some embodiments, the administration of seladelpar or a pharmaceutically acceptable salt thereof results in: (a) an improvement in cholestasis; (b) an improvement in liver injury; and (c) an improvement or prevention of pruritus.

For example, the administration of seladelpar or a pharmaceutically acceptable salt may result in improvement in markers of cholestasis, liver injury, and pruritus. In some embodiments, the administration of seladelpar or a pharmaceutically acceptable salt may result in one or more of: decrease and/or normalization in serum alkaline phosphatase (ALP) level, decrease and/or normalization in serum total bilirubin (TB) level, decrease and/or normalization in serum aspartate aminotransferase (AST) level, decrease and/or normalization in alanine aminotransferase (ALT) level, decrease and/or normalization in gamma-glutamyltransferase (GGT), decrease in pruritus Numerical Rating Scale (NRS), decrease in PBC-40 itch domain, and decrease in 5-D itch scale, wherein said decreases are relative to baseline (especially for patients with baseline NRS≥4), pre-treatment, or at the start of treatment. For example, ALP, TB, AST, ALT, or GGT level may decrease from baseline and/or return close to the normal level. In some embodiments, ALP, TB, AST, ALT, or GGT level may be lower than 1.1, 1.2, 1.3, 1.4, 1.5, 1.6, 1.67, 1.7, 1.8, 1.9, or 2.0 of ULN (upper limit normal) after seladelpar treatment, compared to baseline, pre-treatment, or at the start of treatment.

In some embodiments, the patient has severe pruritus (NRS≥7) at baseline, pre-treatment, or at the start of treatment. In some embodiments, the patient has moderate pruritus (NRS≥4, <7) at baseline, pre-treatment, or at the start of treatment. In some embodiments, the patient has moderate to severe pruritus (NRS≥4) at baseline, pre-treatment, or at the start of treatment. In some embodiments, the patient has no pruritus (NRS=0) at baseline, pre-treatment, or at the start of treatment. In some embodiments, the patient has near no pruritus (NRS=0 or 1) at baseline, pre-treatment, or at the start of treatment. In some embodiments, the patient has clinically significant pruritus (PBC-40≥7) at baseline, pre-treatment, or at the start of treatment. In some embodiments, the patient has severe pruritus (NRS≥7) or clinically significant pruritus (PBC-40 itch domain >7), at baseline, pre-treatment, or at the start of treatment.

In some embodiments, the pruritus NRS may decrease from NRS≥4 to NRS<4 after the administration of seladelpar.

In some embodiments, the patient has moderate to severe (NRS≥4) or severe (NRS>=7) pruritus at baseline, pre-treatment, or at the start of treatment, and administration of seladelpar or a pharmaceutically acceptable salt for six months or more results in complete or near resolution of pruritus (NRS=0 or 1). In some embodiments, the patient has moderate to severe (NRS≥4) or severe (NRS>=7) pruritus at baseline, pre-treatment, or at the start of treatment, and administration of seladelpar or a pharmaceutically acceptable salt for about twelve months results in complete or near resolution of pruritus (NRS=0 or 1). In some embodiments, the patient has moderate to severe (NRS>=4) or severe (NRS>=7) pruritus at baseline, pre-treatment, or at the start of treatment, and administration of seladelpar or a pharmaceutically acceptable salt for six months or more results in complete resolution of pruritus (NRS=0). In some embodiments, the patient has moderate to severe (NRS>=4) or severe (NRS>=7) pruritus at baseline, pre-treatment, or at the start of treatment, and administration of seladelpar or a pharmaceutically acceptable salt for about twelve months results in complete resolution of pruritus (NRS=0).

In some embodiments, the patient has no pruritus (NRS=0) at the baseline, pre-treatment, or at the start of treatment, and does not develop pruritus (NRS=0) after administration of seladelpar or a pharmaceutically acceptable salt for about 12 months.

In some embodiments, the administration results in one or more of an improvement in at least one of sleep disturbance and fatigue.

In some embodiments, the administration results in decrease in decrease in at least one of PBC-40 sleep disturbance domain and fatigue.

In some embodiments, the patient has severe pruritus (NRS≥7) or clinically significant pruritus (PBC-40 itch domain ≥7), at baseline, pre-treatment, or at the start of treatment.

In some embodiments, the administration of seladelpar or a pharmaceutically acceptable salt may result in two or more, three or more, four or more of the previous results. In some embodiments, the administration of seladelpar or a pharmaceutically acceptable salt may result in: normalization in serum alkaline phosphatase (ALP) level and/or decrease in ALP level compared to baseline or pre-treatment, normalization in serum total bilirubin (TB) level or decrease in serum total TB level compared to baseline or pre-treatment, decrease in serum aspartate aminotransferase (AST) level compared to baseline or pre-treatment, decrease in alanine aminotransferase (ALT) level compared to baseline or pre-treatment, decrease in gamma-glutamyltransferase (GGT), and decrease in pruritus Numerical Rating Scale (NRS) (especially for patients with baseline NRS≥4) compared to baseline or pre-treatment. The aforementioned ALP level, TB level, AST level, ALT level, GGT level, and pruritus NRS may be obtained/measured according to methods known in the art.

In some embodiments, such improvement and/or effect of seladelpar may be durable and may be shown even in long-term treatment, for example for up to 3 months, 6 months, 9 months, 12 months, 18 months, 24 months, or 36 months. For example, the administration of seladelpar or a pharmaceutically acceptable salt for up to 3 months, 6 months, 9 months, 12 months, 18 months, 24 months, or 36 months, may result in one or more of: decrease and/or normalization in serum alkaline phosphatase (ALP) level, decrease and/or normalization in serum total bilirubin (TB) level, decrease in serum aspartate aminotransferase (AST) level, decrease in alanine aminotransferase (ALT) level, decrease in gamma-glutamyltransferase (GGT), and decrease in pruritus Numerical Rating Scale (NRS) (especially for patients with baseline NRS≥4).

Patients with PBC compared to healthy subjects were reported to have a distinct metabolic profile and metabolic pathways. With the progression of PBC, increased levels of bile acid were observed while the levels of carnitine and short-chain acylcarnitines (propionyl carnitine and butyryl carnitine) were decreased. Also, an elevation of conjugated primary bile acids, ketone bodies, free fatty acids, long-chain saturated/unsaturated fatty acids and medium-/long-chain acylcarnitines were reported in PBC patients, suggestive of decreased fatty acid oxidation in patients with PBC. Cholestasis is associated with impaired mitochondrial function, resulting in fuel switching from fatty acid O-oxidation towards compensatory glycolysis. Systemic mitochondrial dysfunction was reported in a detailed metabolic study of PBC patients after exercise.

In some embodiments, the administration of seladelpar or a pharmaceutically acceptable salt may result in changes in one or more serum level of metabolism markers, such as the metabolism markers are related to mitochondrial function and/or fatty acid 3-oxidation. Such metabolism markers may be referred to as metabolites in the present disclosure. In some embodiments, the administration of seladelpar or a pharmaceutically acceptable salt may result in increase of serum level of one or more of carnitine, acetylcarnitine, other acylcarnitines (i.e., short-, medium- and long-chain acylcarnitines), N,N,N-trimethyl-5-aminovalerate (TMAVA), and branched-chain amino acid catabolites. Said acylcarnitine may be one or more selected from propionyl carnitine (C3:0), butyryl carnitine (C4:0), isobutyryl carnitine (C5:0), valeryl carnitine (C6:0), isovaleryl carnitine (C5:0), 2-methylbutyryl carnitine (C4:0), 3-hydroxybutyryl carnitine (C4:0), hexanoyl carnitine (C6:0), octanoyl carnitine (C8:0), decanoyl carnitine (C10:0), dodecanoyl carnitine (C12:0), myristoyl carnitine (C14:0), palmitoyl carnitine (C16:0), linoleoyl carnitine (C18:2), oleoyl carnitine (C18:1), and stearoyl carnitine (C18:0). In some embodiments, the acylcarnitine is 3-hydroxybutyryl carnitine. Said branched-chain amino acid catabolites may be one or more selected from 2,3-dihydroxy-2-methylbutyrate, 3-methyl-2-oxobutyrate, and 3-methyl-2-oxovalerate and 4-methyl-2-oxopentanoate. In some embodiments, the branched-chain amino acid catabolites is 2,3-dihydroxy-2-methylbutyrate.

In some embodiments, the administration of seladelpar or a pharmaceutically acceptable salt may result in decrease of serum level of dicarboxylates, saturated and unsaturated fatty acids, and lipid-derived inflammatory mediators, such as ceramides. Said dicarboxylates may be one or more selected from dodecanedioate (C12-DC), sebacate (C10-DC), octadecanedioate (C18-DC), tetradecanedioate (C14-DC), hexadecanedioate (C16-DC), and dodecenedioate (C12:1-DC). In some embodiments, dicarboxylates may be sebacate, dodecanedioate, or tetradecanediote.

In some embodiments, the present disclosure provides methods of treating PBC, comprising administering a therapeutically effective amount of seladelpar or a pharmaceutically acceptable salt thereof, wherein administration of seladelpar or a pharmaceutically acceptable salt thereof results in (a) serum level increase of serum level of one or more of carnitine, acetylcarnitine, other acylcarnitines (i.e., short-, medium- and long-chain acylcarnitines), N,N,N-trimethyl-5-aminovalerate (TMAVA), and branched-chain amino acid catabolites; and/or (b) decrease of serum level of dicarboxylates, saturated and unsaturated fatty acids, and lipid-derived inflammatory mediators, such as ceramides. In some embodiments, the administration of seladelpar increases the expression of OCTN2 and genes involved in mitochondrial carnitine shuttle (CPT1A, CPT2, CACT, and CRAT).

In some embodiments, the present disclosure provides methods of increasing serum level of one or more of carnitine, acetylcarnitine, other acylcarnitines (i.e., short-, medium- and long-chain acylcarnitines), N,N,N-trimethyl-5-aminovalerate (TMAVA), and branched-chain amino acid catabolites in a subject in need thereof (e.g., a PBC patient), comprising administering a therapeutically effective amount of seladelpar or a pharmaceutically acceptable salt thereof. In some embodiments, the present disclosure provides methods of decreasing serum level of one or more of dicarboxylates, saturated and unsaturated fatty acids, and lipid-derived inflammatory mediators, such as ceramides, in a subject in need thereof (e.g., a PBC patient), comprising administering a therapeutically effective amount of seladelpar or a pharmaceutically acceptable salt thereof.

In some embodiments, the present disclosure provides methods of increasing expression level of one or more genes selected from OCTN2, CPT1A, CPT2, CACT, and CRAT, comprising administering a therapeutically effective amount of seladelpar or a pharmaceutically acceptable salt thereof. In some embodiments, the present disclosure provides methods of increasing expression level of FGF21, comprising administering a therapeutically effective amount of seladelpar or a pharmaceutically acceptable salt thereof. In some embodiments, the present disclosure provides methods of decreasing expression level of IL-31, comprising administering a therapeutically effective amount of seladelpar or a pharmaceutically acceptable salt thereof.

In some embodiments, the present disclosure provides methods of treating PBC, comprising administering a therapeutically effective amount of seladelpar or a pharmaceutically acceptable salt thereof, and monitoring the serum level of one or more of metabolites selected from the group consisting of: carnitine, acetylcarnitine, other acylcarnitines (i.e., short-, medium- and long-chain acylcarnitines), N,N,N-trimethyl-5-aminovalerate (TMAVA), branched-chain amino acid catabolites, dicarboxylates, saturated and unsaturated fatty acids, and lipid-derived inflammatory mediators, such as ceramides. By monitoring the metabolites, the progress of the treatment of PBC may be determined. In some embodiments, the present disclosure provides methods of treating PBC, comprising administering a therapeutically effective amount of seladelpar or a pharmaceutically acceptable salt thereof, and monitoring an expression level of one or more genes selected from OCTN2, CPT1A, CPT2, CACT, and CRAT.

In some embodiments, the present disclosure provides methods of determining activities of seladelpar and/or progress of seladelpar treatment in a subject, comprising measuring the serum level of one or more of metabolites selected from the group consisting of: carnitine, acetylcarnitine, other acylcarnitines (i.e., short-, medium- and long-chain acylcarnitines), N,N,N-trimethyl-5-aminovalerate (TMAVA), branched-chain amino acid catabolites, dicarboxylates, saturated and unsaturated fatty acids, and lipid-derived inflammatory mediators, such as ceramides in the subject. In some embodiments, the present disclosure provides methods of determining activities of seladelpar and/or progress of seladelpar treatment in a subject, comprising measuring the expression level of one or more genes selected from OCTN2, CPT1A, CPT2, CACT, and CRAT in the subject.

In some embodiments, seladelpar or a pharmaceutically acceptable salt thereof to a patient, to decrease serum level of at least one cholesterol level in a patient in need thereof. Said cholesterol level may be total cholesterol level, low-density lipoprotein (LDL) level, and/or triglyceride level. In some embodiments, at least one statin may be further administered to the patient. In some embodiments, statin is not administered to the patient. Said at least one statin may be selected from atorvastatin, cerivastatin, fluvastatin, lovastatin, pitavastatin, pravastatin, rosuvastatin, simvastatin, cerivastatin, analogs thereof, and a combination thereof.

### Patient Population and Drug-Drug Interaction

In some embodiments, the present disclosure provides methods of treating a liver disease or methods of administering seladelpar or a pharmaceutically acceptable salt, in certain groups of patients, in certain situations, or with regard to co-administration of drugs other than seladelpar or a pharmaceutically acceptable salt thereof.

### Decompensated Cirrhosis

Seladelpar may not be recommended in patients who have or develop decompensated cirrhosis (e.g., ascites, variceal bleeding, hepatic encephalopathy), from the safety and efficacy concern. In some embodiments, the present disclosure provides a method of treating a liver disease in a patient in need thereof, the method comprising administering a therapeutically effective amount of seladelpar or a pharmaceutically acceptable salt thereof, wherein the patient does not have decompensated cirrhosis. In some embodiments, the present disclosure provides a method of administering seladelpar therapy in a subject in need thereof, comprising administration of a therapeutically effective amount of seladelpar or a pharmaceutically acceptable salt thereof, wherein the patient does not have decompensated cirrhosis.

The liver disease may be primary biliary cholangitis (PBC). In some embodiments, the liver disease may be primary sclerosing cholangitis (PSC) or nonalcoholic steatohepatitis (NASH). Seladelpar or a pharmaceutically acceptable salt thereof may be administered orally. The patients may be also administered a therapeutically effective amount of ursodeoxycholic acid (UDCA). The patient may have an inadequate response to UDCA. Or the patient may be intolerant of UDCA. Seladelpar or a pharmaceutically acceptable salt thereof may be administered as a monotherapy. In some embodiments, the patient may be administered a therapeutically effective amount of seladelpar lysine. Said seladelpar lysine may be seladelpar lysine dihydrate. The amount of seladelpar may be 5 mg/day or 10 mg/day.

Patients with decompensated cirrhosis may have ascites, variceal bleeding, hepatic encephalopathy, or jaundice. In some embodiments, patients with severe hepatic impairment (Child-Pugh C) are considered to have decompensated cirrhosis and will not be recommended seladelpar. In some embodiments, patients with moderate or severe hepatic impairment (Child-Pugh B or C) are not recommended seladelpar. On the other hand, no dosage adjustment may be recommended for patients with mild hepatic impairment (Child-Pugh A), and 5 mg/day or 10 mg/day of seladelpar may be administered to patients with mild hepatic impairment (Child-Pugh A).

In some embodiments, reduced dosage of seladelpar may be administered to patients with moderate hepatic impairment (Child-Pugh B). For example, 5 mg/day of seladelpar may be administered to patients with moderate hepatic impairment (Child-Pugh B).

In some embodiments, liver function or hepatic impairment of the patient are monitored and/or tested for decompensated cirrhosis. In some embodiments, patients with cirrhosis are monitored and/or tested for evidence of decompensation, such as jaundice, ascites, variceal bleeding, or hepatic encephalopathy. Discontinuing the administration of seladelpar may be considered if the patient progresses to moderate or severe hepatic impairment (Child-Pugh B or C).

### Drug-Induced Liver Injury and Liver Test Abnormalities

Seladelpar may be associated with drug-induced liver injury. In some embodiments, the present disclosure provides a method of treating a liver disease in a patient in need thereof, the method comprising administering a therapeutically effective amount of seladelpar or a pharmaceutically acceptable salt thereof and monitoring and/or testing the patient for signs or symptoms of liver injury. In some embodiments, the present disclosure provides a method of treating a liver disease in a patient in need thereof, the method comprising administering a therapeutically effective amount of seladelpar or a pharmaceutically acceptable salt thereof and testing the patient for signs of liver injury or abnormalities. In some embodiments, the present disclosure provides a method of administering seladelpar therapy in a subject in need thereof, comprising administration of a therapeutically effective amount of seladelpar or a pharmaceutically acceptable salt thereof and monitoring and/or testing the patient for signs or symptoms of liver injury. In some embodiments, the present disclosure provides a method of administering seladelpar therapy in a subject in need thereof, the method comprising administering a therapeutically effective amount of seladelpar or a pharmaceutically acceptable salt thereof and testing the patient for liver injury or abnormalities.

The liver disease may be primary biliary cholangitis (PBC). In some embodiments, the liver disease may be primary sclerosing cholangitis (PSC) or nonalcoholic steatohepatitis (NASH). Seladelpar or a pharmaceutically acceptable salt thereof may be administered orally. The patients may be also administered a therapeutically effective amount of ursodeoxycholic acid (UDCA). The patient may have an inadequate response to UDCA. Or the patient may be intolerant of UDCA. Seladelpar or a pharmaceutically acceptable salt thereof may be administered as a monotherapy. In some embodiments, the patient may be administered a therapeutically effective amount of seladelpar lysine. Said seladelpar lysine may be seladelpar lysine dihydrate. The amount of seladelpar may be 5 mg/day or 10 mg/day.

Seladelpar has been associated with dose-related increases in serum transaminase (i.e., aspartate aminotransferase (AST) and alanine aminotransferase (ALT)) levels greater than 3-times upper limit of normal (ULN) in PBC patients receiving 50 mg once daily and 200 mg once daily. Transaminase levels returned to pretreatment levels upon seladelpar discontinuation. On the other hand, seladelpar 5 mg/day or 10 mg once daily did not show a similar pattern for increases in transaminase levels.

To prevent or stop drug-induced liver injury, baseline clinical and laboratory assessments may be obtained at initiation of treatment with seladelpar and monitored, tested, or followed up thereafter, for example, according to routine patient management. For example, serum levels of ALT, AST, total bilirubin (TB), and/or alkaline phosphatase (ALP) may be obtained. Seladelpar treatment may be stopped or interrupted if signs of liver injury or abnormalities are present, for example if the liver tests (ALT, AST, total bilirubin (TB), and/or alkaline phosphatase (ALP)) worsen, if other indications of impaired liver function (e.g., increase in INR) are observed, or if the patient develops signs and symptoms consistent with clinical hepatitis (e.g., jaundice, right upper quadrant pain or tenderness, eosinophilia, fatigue, nausea, fever, rash). Seladelpar treatment may also be stopped or interrupted if signs of muscle injury or abnormalities are present, for example if muscle tests (creatine phosphokinase) worsen or there are clinical signs of muscle injury (e.g. muscle pain or tenderness).

### Fractures

It has been found that chance of fractures may increase for seladelpar-treated patients. In some embodiments, the present disclosure provides a method of treating a liver disease in a patient in need thereof, comprising administering a therapeutically effective amount of seladelpar or a pharmaceutically acceptable salt thereof and monitoring and/or testing and optionally treating the patient for bone density and/or bone health. In some embodiments, the present disclosure provides a method of providing seladelpar therapy to a patient in need thereof, comprising administration of a therapeutically effective amount of seladelpar, further comprising monitoring and/or testing and optionally treating the patient for bone density and/or bone health.

The liver disease may be primary biliary cholangitis (PBC). In some embodiments, the liver disease may be primary sclerosing cholangitis (PSC) or nonalcoholic steatohepatitis (NASH). Seladelpar or a pharmaceutically acceptable salt thereof may be administered orally. The patients may be also administered a therapeutically effective amount of ursodeoxycholic acid (UDCA). Or the patient may be intolerant of, or has an inadequate response to UDCA. Seladelpar or a pharmaceutically acceptable salt thereof may be administered as a monotherapy. In some embodiments, the patient may be administered a therapeutically effective amount of seladelpar lysine. Said seladelpar lysine may be seladelpar lysine dihydrate. The amount of seladelpar may be 5 mg/day or 10 mg/day.

In some embodiments, the risk of fracture may be considered in the care of patients treated with seladelpar. The bone health may be monitored or followed up according to current standards of care. In some embodiments, patients may be informed that seladelpar may increase the risk of bone fractures and/or advised to call their healthcare provide to report any fractures.

### Biliary Obstruction

Use of seladelpar may be avoided in patients with complete biliary obstruction. In some embodiments, the present disclosure provides a method of treating a liver disease in a patient in need thereof, the method comprising administering a therapeutically effective amount of seladelpar or a pharmaceutically acceptable salt thereof, wherein the patient does not have complete biliary obstruction. In some embodiments, the present disclosure provides a method of administering seladelpar therapy in a subject in need thereof, comprising administration of a therapeutically effective amount of seladelpar or a pharmaceutically acceptable salt thereof, wherein the patient does not have complete biliary obstruction.

The liver disease may be primary biliary cholangitis (PBC). In some embodiments, the liver disease may be primary sclerosing cholangitis (PSC) or nonalcoholic steatohepatitis (NASH). Seladelpar or a pharmaceutically acceptable salt thereof may be administered orally. The patients may be also administered a therapeutically effective amount of ursodeoxycholic acid (UDCA). Or the patient may be intolerant of, or has an inadequate response to UDCA. Seladelpar or a pharmaceutically acceptable salt thereof may be administered as a monotherapy. In some embodiments, the patient may be administered a therapeutically effective amount of seladelpar lysine. Said seladelpar lysine may be seladelpar lysine dihydrate. The amount of seladelpar may be 5 mg/day or 10 mg/day.

If biliary obstruction is suspected, seladelpar treatment may be stopped and/or treated as clinically indicated. In some embodiments, patients are instructed to immediately report any signs or symptoms of biliary obstruction (e.g., right upper quadrant pain, jaundice) to their healthcare provider, so that seladelpar treatment can be interrupted while the patient is being evaluated, monitored, and/or tested for biliary obstruction.

### Other Populations

In some embodiments, patients (i.e., PBC patients) or any patients described herein, may belong to one or more of the following population. (a) the patient is less than 65 years old, or 65 years old or older; (b) the patient was less than 50 years old at PBC diagnosis, or 50 years old or older; (c) the patient is white, Asian, black, Latino, or non-Latino; (d) the patient is male or female; (e) the patient is from North America, Europe, or rest-of-world; (f) the patient has serum ALP level of <350 U/L or ≥350 U/L at baseline; (g) the patient has Total Bilirubin (TB) level of <0.6×ULN or ≥0.6×ULN at baseline; (h) the patient has Total Bilirubin (TB) level of <1×ULN or ≥1×ULN at baseline; (i) the patient has pruritus NRS of <4 or ≥4 at baseline; or (j) the patient has cirrhosis or does not have cirrhosis.

### Drug-Drug Interaction

Seladelpar, in vitro, has been reported to a substrate of CYP2C9, CYP2C8, CYP3A4, and the transporters BCRP, P-gp, and OAT3, and drugs related to these pathways may affect administration of seladelpar. On the other hand, seladelpar, in vitro, has been reported not to be a substrate of MATE1, MATE2-K, OAT1, OCT1, or OCT2.

Probenecid is known to be an OAT3 and OATP1B inhibitor, and it has been found that concomitant administration of seladelpar with probenecid can increase seladelpar exposure. It has been also found that concomitant administration of seladelpar with a strong CYP2C9 inhibitor can increase seladelpar exposure. In some embodiments, the present disclosure provides a method of treating a liver disease in a patient in need thereof, the method comprising administering a therapeutically effective amount of seladelpar or a pharmaceutically acceptable salt thereof, while avoiding co-administration of an OAT3 inhibitor or a strong CYP2C9 inhibitor. In some embodiments, the present disclosure provides a method of treating a liver disease in a patient in need thereof, the method comprising administering a therapeutically effective amount of seladelpar or a pharmaceutically acceptable salt thereof, while avoiding co-administration of an OAT3 inhibitor. In some embodiments, the OAT3 inhibitor may be probenecid. In some embodiments, the present disclosure provides a method of treating a liver disease in a patient in need thereof, the method comprising administering a therapeutically effective amount of seladelpar or a pharmaceutically acceptable salt thereof, while avoiding co-administration of probenecid. In some embodiments, the present disclosure provides a method of treating a liver disease in a patient in need thereof, the method comprising administering a therapeutically effective amount of seladelpar or a pharmaceutically acceptable salt thereof, while not administering of probenecid. In some embodiments, the present disclosure provides a method of treating a liver disease in a patient in need thereof, the method comprising administering a therapeutically effective amount of seladelpar or a pharmaceutically acceptable salt thereof, and advising the patient that co-administering with a strong CYP2C9 inhibitor may result in adverse effects. In some embodiments, the present disclosure provides a method of treating a liver disease in a patient in need thereof, the method comprising administering a therapeutically effective amount of seladelpar or a pharmaceutically acceptable salt thereof, while avoiding co-administration of a strong CYP2C9 inhibitor. In some embodiments, the present disclosure provides a method of treating a liver disease in a patient in need thereof, the method comprising administering a therapeutically effective amount of seladelpar or a pharmaceutically acceptable salt thereof, while avoiding co-administration of a sulfaphenazole, a strong CYP2C9 inhibitor. In some embodiments, the present disclosure provides a method of treating a liver disease in a patient in need thereof, the method comprising administering a therapeutically effective amount of seladelpar or a pharmaceutically acceptable salt thereof, and while monitoring for adverse effects from co-administering with a strong CYP2C9 inhibitor. In some embodiments, the strong CYP2C9 inhibitor is sulfaphenazole. In some embodiments, the present disclosure provides a method of administering seladelpar therapy in a subject in need thereof, comprising administration of a therapeutically effective amount of seladelpar or a pharmaceutically acceptable salt thereof, while avoiding co-administration of an OAT3 inhibitor or a strong CYP2C9 inhibitor. In some embodiments, the present disclosure provides a method of administering seladelpar therapy in a subject in need thereof, comprising administration of a therapeutically effective amount of seladelpar or a pharmaceutically acceptable salt thereof, while avoiding co-administration of an OAT3 inhibitor. In some embodiments, the OAT3 inhibitor may be probenecid. In some embodiments, the present disclosure provides a method of administering seladelpar therapy in a subject in need thereof, comprising administration of a therapeutically effective amount of seladelpar or a pharmaceutically acceptable salt thereof, while avoiding co-administration of probenecid. In some embodiments, the present disclosure provides a method of administering seladelpar therapy in a subject in need thereof, comprising administration of a therapeutically effective amount of seladelpar or a pharmaceutically acceptable salt thereof, while not administering probenecid. In some embodiments, the present disclosure provides a method of administering seladelpar therapy in a subject in need thereof, comprising administration of a therapeutically effective amount of seladelpar or a pharmaceutically acceptable salt thereof, and advising the patient that co-administering with a strong CYP2C9 inhibitor may result in adverse effects. In some embodiments, the present disclosure provides a method of administering seladelpar therapy in a subject in need thereof, comprising administration of a therapeutically effective amount of seladelpar or a pharmaceutically acceptable salt thereof, while avoiding co-administration of a strong CYP2C9 inhibitor. In some embodiments, the present disclosure provides a method of administering seladelpar therapy in a subject in need thereof, comprising administration of a therapeutically effective amount of seladelpar or a pharmaceutically acceptable salt thereof, while avoiding co-administration of sulfaphenazole, a strong CYP2C9 inhibitor. In some embodiments, the present disclosure provides a method of administering seladelpar therapy in a subject in need thereof, the method comprising administering a therapeutically effective amount of seladelpar or a pharmaceutically acceptable salt thereof, and while monitoring for adverse effects from co-administering with a strong CYP2C9 inhibitor. In some embodiments, the strong CYP2C9 inhibitor is sulfaphenazole.

The liver disease may be primary biliary cholangitis (PBC). In some embodiments, the liver disease may be primary sclerosing cholangitis (PSC) or nonalcoholic steatohepatitis (NASH). Seladelpar or a pharmaceutically acceptable salt thereof may be administered orally. The patients may be also administered a therapeutically effective amount of ursodeoxycholic acid (UDCA). Or the patient may be intolerant of, or has an inadequate response to UDCA. Seladelpar or a pharmaceutically acceptable salt thereof may be administered as a monotherapy. In some embodiments, the patient may be administered a therapeutically effective amount of seladelpar lysine. Said seladelpar lysine may be seladelpar lysine dihydrate. The amount of seladelpar may be 5 mg/day or 10 mg/day.

In some embodiments, the OAT3 inhibitor may be gemfibrozil, probenecid, or teriflunomide. In some embodiments, the OAT inhibitor is probenecid.

The strong inhibitors may be drugs that increase the AUC of sensitive index substrates of a given metabolic pathway ≥5-fold. In some embodiments, the strong CYP2C9 inhibitor is selected from ritonavir, fluconazole, fluvoxamine, amentoflavone, or sulphaphenazole. In some embodiments, the strong CYP2C9 inhibitor is sulfaphenazole.

In some embodiments, if the patient has been or is being treated with an OAT3 inhibitor or a strong CYP2C9 inhibitors, administration of the OAT3 inhibitor or the strong CYP2C9 inhibitor may be discontinued prior to the administration of seladelpar. For example, administration of the OAT3 inhibitor or the strong CYP2C9 inhibitor may be discontinued at least 1 day, 3 days, 7 days, 2 weeks, or 4 weeks before the administration of seladelpar.

It has been found that concomitant administration of seladelpar with rifampin, an inducer of metabolizing enzymes, may reduce the systemic exposure of seladelpar and may result in delayed or suboptimal biochemical response (e.g., ALP and bilirubin). In some embodiments, the present disclosure provides a method of treating a liver disease in a patient in need thereof, the method comprising administering a therapeutically effective amount of seladelpar or a pharmaceutically acceptable salt thereof, and advising the patient that co-administering with rifampin may result in delayed or suboptimal biochemical response (e.g., ALP and bilirubin) of the seladelpar therapy. In some embodiments, the present disclosure provides a method of administering seladelpar therapy in a subject in need thereof, comprising administration of a therapeutically effective amount of seladelpar or a pharmaceutically acceptable salt thereof, and advising the patient that co-administering with rifampin may result in delayed or suboptimal biochemical response (e.g., ALP and bilirubin) of the seladelpar therapy.

The liver disease may be primary biliary cholangitis (PBC). In some embodiments, the liver disease may be primary sclerosing cholangitis (PSC) or nonalcoholic steatohepatitis (NASH). Seladelpar or a pharmaceutically acceptable salt thereof may be administered orally. The patients may be also administered a therapeutically effective amount of ursodeoxycholic acid (UDCA). Or the patient may be intolerant of, or has an inadequate response to UDCA. Seladelpar or a pharmaceutically acceptable salt thereof may be administered as a monotherapy. In some embodiments, the patient may be administered a therapeutically effective amount of seladelpar lysine. Said seladelpar lysine may be seladelpar lysine dihydrate. The amount of seladelpar may be 5 mg/day or 10 mg/day.

In some embodiments, the patients may be monitored or followed up for delayed or suboptimal biochemical response (e.g., ALP and bilirubin) of the seladelpar therapy if the patient is co-administered with rifampin.

It has been found that concomitant use with a drug that is a dual moderate inhibitor of CYP2C9 and moderate to strong inhibitor of CYP3A4 may increase exposure of seladelpar. Additionally, cyclosporine is known to be an OATP1B1, OATP1B3, BCRP, and CYP3A inhibitor and it has been also found that concomitant use with cyclosporine may increase exposure of seladelpar.

In some embodiments, the present disclosure provides a method of treating a liver disease in a patient in need thereof, the method comprising administering a therapeutically effective amount of seladelpar or a pharmaceutically acceptable salt thereof, and advising the patient that co-administering with a dual moderate CYP2C9 and moderate to strong CYP3A4 inhibitor or a BCRP inhibitor may result in adverse effects. In some embodiments, the present disclosure provides a method of treating a liver disease in a patient in need thereof, the method comprising administering a therapeutically effective amount of seladelpar or a pharmaceutically acceptable salt thereof, and advising the patient that co-administering with a dual moderate CYP2C9 and moderate to strong CYP3A4 inhibitor may result in adverse effects. In some embodiments, the present disclosure provides a method of treating a liver disease in a patient in need thereof, the method comprising administering a therapeutically effective amount of seladelpar or a pharmaceutically acceptable salt thereof, and monitoring for adverse effects from co-administering with a dual moderate CYP2C9 and moderate to strong CYP3A4 inhibitor. In some embodiments, the present disclosure provides a method of treating a liver disease in a patient in need thereof, the method comprising administering a therapeutically effective amount of seladelpar or a pharmaceutically acceptable salt thereof, and avoiding co-administering with a dual moderate CYP2C9 and moderate to strong CYP3A4 inhibitor. In some embodiments, the dual moderate CYP2C9 and moderate to strong CYP3A4 inhibitor is fluconazole. In some embodiments, the present disclosure provides a method of treating a liver disease in a patient in need thereof, the method comprising administering a therapeutically effective amount of seladelpar or a pharmaceutically acceptable salt thereof, and avoiding co-administering with fluconazole. The liver disease may be primary biliary cholangitis (PBC). In some embodiments, the liver disease may be primary sclerosing cholangitis (PSC) or nonalcoholic steatohepatitis (NASH). Seladelpar or a pharmaceutically acceptable salt thereof may be administered orally. The patients may be also administered a therapeutically effective amount of ursodeoxycholic acid (UDCA). Or the patient may be intolerant of, or has an inadequate response to UDCA. Seladelpar or a pharmaceutically acceptable salt thereof may be administered as a monotherapy. In some embodiments, the patient may be administered a therapeutically effective amount of seladelpar lysine. Said seladelpar lysine may be seladelpar lysine dihydrate. The amount of seladelpar may be 5 mg/day or 10 mg/day.

In some embodiments, the present disclosure provides a method of treating a liver disease in a patient in need thereof, the method comprising administering a therapeutically effective amount of seladelpar or a pharmaceutically acceptable salt thereof, and advising the patient that co-administering with a BCRP inhibitor may result in adverse effects. In some embodiments, the present disclosure provides a method of treating a liver disease in a patient in need thereof, the method comprising administering a therapeutically effective amount of seladelpar or a pharmaceutically acceptable salt thereof, and avoiding co-administering with a BCRP inhibitor may result in adverse effects. In some embodiments, the present disclosure provides a method of treating a liver disease in a patient in need thereof, the method comprising administering a therapeutically effective amount of seladelpar or a pharmaceutically acceptable salt thereof, and advising the patient that co-administering with cyclosporine may result in adverse effects. In some embodiments, the present disclosure provides a method of treating a liver disease in a patient in need thereof, the method comprising administering a therapeutically effective amount of seladelpar or a pharmaceutically acceptable salt thereof, and monitoring for the adverse effects from co-administering with cyclosporine. The liver disease may be primary biliary cholangitis (PBC). In some embodiments, the liver disease may be primary sclerosing cholangitis (PSC) or nonalcoholic steatohepatitis (NASH). Seladelpar or a pharmaceutically acceptable salt thereof may be administered orally. The patients may be also administered a therapeutically effective amount of ursodeoxycholic acid (UDCA). Or the patient may be intolerant of, or has an inadequate response to UDCA. Seladelpar or a pharmaceutically acceptable salt thereof may be administered as a monotherapy. In some embodiments, the patient may be administered a therapeutically effective amount of seladelpar lysine. Said seladelpar lysine may be seladelpar lysine dihydrate. The amount of seladelpar may be 5 mg/day or 10 mg/day.

In some embodiments, the present disclosure provides a method of administering seladelpar therapy in a subject in need thereof, comprising administration of a therapeutically effective amount of seladelpar or a pharmaceutically acceptable salt thereof, advising the patient that co-administering with a dual moderate CYP2C9 and moderate to strong CYP3A4 inhibitor or a BCRP inhibitor may result in adverse effects. In some embodiments, the present disclosure provides a method of administering seladelpar therapy in a subject in need thereof, comprising administration of a therapeutically effective amount of seladelpar or a pharmaceutically acceptable salt thereof, advising the patient that co-administering with a dual moderate CYP2C9 and moderate to strong CYP3A4 inhibitor may result in adverse effects. In some embodiments, the present disclosure provides a method of administering seladelpar therapy in a subject in need thereof, the method comprising administering a therapeutically effective amount of seladelpar or a pharmaceutically acceptable salt thereof, and monitoring for adverse effects from co-administering with a dual moderate CYP2C9 and moderate to strong CYP3A4 inhibitor. In some embodiments, the present disclosure provides a method of administering seladelpar therapy in a subject in need thereof, the method comprising administering a therapeutically effective amount of seladelpar or a pharmaceutically acceptable salt thereof, and avoiding co-administering with a dual moderate CYP2C9 and moderate to strong CYP3A4 inhibitor. In some embodiments, the dual moderate CYP2C9 and moderate to strong CYP3A4 inhibitor is fluconazole. In some embodiments, the present disclosure provides a method of administering seladelpar therapy in a subject in need thereof, the method comprising administering a therapeutically effective amount of seladelpar or a pharmaceutically acceptable salt thereof, and avoiding co-administering with fluconazole.

In some embodiments, the present disclosure provides a method of administering seladelpar therapy in a subject in need thereof, comprising administration of a therapeutically effective amount of seladelpar or a pharmaceutically acceptable salt thereof, advising the patient that co-administering with a BCRP inhibitor may result in adverse effects. In some embodiments, the present disclosure provides a method of administering seladelpar therapy in a subject in need thereof, comprising administration of a therapeutically effective amount of seladelpar or a pharmaceutically acceptable salt thereof, avoiding co-administering with a BCRP inhibitor. In some embodiments, the present disclosure provides a method of administering seladelpar therapy in a subject in need thereof, comprising administration of a therapeutically effective amount of seladelpar or a pharmaceutically acceptable salt thereof, advising the patient that co-administering with cyclosporine may result in adverse effects. In some embodiments, the present disclosure provides a method of administering seladelpar therapy in a subject in need thereof, the method comprising administering a therapeutically effective amount of seladelpar or a pharmaceutically acceptable salt thereof, and monitoring for the adverse effects from co-administering with cyclosporine. In some embodiments, the present disclosure provides a method of administering seladelpar therapy in a subject in need thereof, comprising administration of a therapeutically effective amount of seladelpar or a pharmaceutically acceptable salt thereof, avoiding co-administering with cyclosporine.

In some embodiments, the present disclosure provides a method of treating a liver disease in a patient in need thereof, the method comprising administering a therapeutically effective amount of seladelpar or a pharmaceutically acceptable salt thereof, and advising the patient that co-administering with a dual or multiple inhibitors of OATP1B1, OATP1B3, BCRP inhibitors, and/or OAT3 may result in adverse effects. In some embodiments, the present disclosure provides a method of treating a liver disease in a patient in need thereof, the method comprising administering a therapeutically effective amount of seladelpar or a pharmaceutically acceptable salt thereof, and avoiding co-administering with a dual or multiple inhibitors of OATP1B1, OATP1B3, BCRP inhibitors, and/or OAT3. In some embodiments, the present disclosure provides a method of treating a liver disease in a patient in need thereof, the method comprising administering a therapeutically effective amount of seladelpar or a pharmaceutically acceptable salt thereof, and advising the patient that co-administering with cyclosporine may result in adverse effects. In some embodiments, the present disclosure provides a method of treating a liver disease in a patient in need thereof, the method comprising administering a therapeutically effective amount of seladelpar or a pharmaceutically acceptable salt thereof, and monitoring for the adverse effects from co-administering with cyclosporine. The liver disease may be primary biliary cholangitis (PBC). In some embodiments, the liver disease may be primary sclerosing cholangitis (PSC) or nonalcoholic steatohepatitis (NASH). Seladelpar or a pharmaceutically acceptable salt thereof may be administered orally. The patients may be also administered a therapeutically effective amount of ursodeoxycholic acid (UDCA). Or the patient may be intolerant of, or has an inadequate response to UDCA. Seladelpar or a pharmaceutically acceptable salt thereof may be administered as a monotherapy. In some embodiments, the patient may be administered a therapeutically effective amount of seladelpar lysine. Said seladelpar lysine may be seladelpar lysine dihydrate. The amount of seladelpar may be 5 mg/day or 10 mg/day.

In some embodiments, the present disclosure provides a method of administering seladelpar therapy in a subject in need thereof, comprising administration of a therapeutically effective amount of seladelpar or a pharmaceutically acceptable salt thereof, advising the patient that co-administering with a dual or multiple inhibitors of OATP1B1, OATP1B3, BCRP inhibitors, and/or OAT3 may result in adverse effects. In some embodiments, the present disclosure provides a method of administering seladelpar therapy in a subject in need thereof, the method comprising administering a therapeutically effective amount of seladelpar or a pharmaceutically acceptable salt thereof, and monitoring for the adverse effects from co-administering with a dual or multiple inhibitors of OATP1B1, OATP1B3, BCRP inhibitors, and/or OAT3. In some embodiments, the present disclosure provides a method of administering seladelpar therapy in a subject in need thereof, comprising administration of a therapeutically effective amount of seladelpar or a pharmaceutically acceptable salt thereof, advising the patient that co-administering with cyclosporine may result in adverse effects. In some embodiments, the present disclosure provides a method of administering seladelpar therapy in a subject in need thereof, the method comprising administering a therapeutically effective amount of seladelpar or a pharmaceutically acceptable salt thereof, and monitoring for the adverse effects from co-administering with cyclosporine.

In some embodiments, the present disclosure provides a method of administering seladelpar therapy in a subject in need thereof, the method comprising administering a therapeutically effective amount of seladelpar or a pharmaceutically acceptable salt thereof, and avoiding co-administering with a BCRP inhibitor. In some embodiments, the present disclosure provides a method of administering seladelpar therapy in a subject in need thereof, comprising administration of a therapeutically effective amount of seladelpar or a pharmaceutically acceptable salt thereof, advising the patient that co-administering with a BCRP inhibitor may result in adverse effects. In some embodiments, the present disclosure provides a method of administering seladelpar therapy in a subject in need thereof, comprising administration of a therapeutically effective amount of seladelpar or a pharmaceutically acceptable salt thereof, advising the patient that co-administering with cyclosporine may result in adverse effects. In some embodiments, the present disclosure provides a method of administering seladelpar therapy in a subject in need thereof, the method comprising administering a therapeutically effective amount of seladelpar or a pharmaceutically acceptable salt thereof, and monitoring for the adverse effects from co-administering with cyclosporine.

In some embodiments, the patients may be monitored or followed up for adverse effects when co-administering seladelpar with the dual moderate CYP2C9 and moderate to strong CYP3A4 inhibitor, or a BCRP inhibitor. The adverse effects may include headache, abdominal pain, nausea, abdominal distension, fracture, or dizziness.

The strong inhibitors may be drugs that increase the AUC of sensitive index substrates of a given metabolic pathway ≥5-fold, and the moderate inhibitors may be drugs that increase the AUC of sensitive index substrates of a given metabolic pathway ≥2- to <5-fold. In some embodiments, the dual moderate CYP2C9 and moderate to strong CYP3A4 inhibitor is fluconazole or amiodarone. In some embodiments, the dual moderate CYP2C9 and moderate to strong CYP3A4 inhibitor is fluconazole.

In some embodiments, the BCRP inhibitor may be curcumin, cyclosporine, darolutamide, eltrombopag, febuxostat, fostamatinib, rolapitant, sofosbuvir, velpatasvir, voxilaprevir, or teriflunomide. In some embodiments, the BCRP inhibitor is cyclosporine.

As described herein, seladelpar is a CYP2C9 and CYP3A4 substrate. Increased seladelpar AUC is expected in patients who are CYP2C9 poor metabolizers with concomitant use of a moderate to strong CYP3A4 inhibitor. The prevalence of CYP2C9 poor metabolizers is approximately 2 to 3% in White populations, 0.5 to 4% in Asian populations, and <1% in African American populations. In some embodiments, the present disclosure provides a method of treating a liver disease in a patient in need thereof, wherein the patient is a poor metabolizer of CYP2C9, the method comprising administering a therapeutically effective amount of seladelpar or a pharmaceutically acceptable salt thereof, and advising the patient that co-administering with a moderate to strong CYP3A4 inhibitor may result in increased exposure of seladelpar. In some embodiments, the present disclosure provides a method of administering seladelpar therapy in a subject in need thereof, wherein the patient is a poor metabolizer of CYP2C9, the method comprising administration of a therapeutically effective amount of seladelpar or a pharmaceutically acceptable salt thereof, and advising the patient that co-administering with a moderate to strong CYP3A4 inhibitor may result in increased exposure of seladelpar.

The liver disease may be primary biliary cholangitis (PBC). In some embodiments, the liver disease may be primary sclerosing cholangitis (PSC) or nonalcoholic steatohepatitis (NASH). Seladelpar or a pharmaceutically acceptable salt thereof may be administered orally. The patients may be also administered a therapeutically effective amount of ursodeoxycholic acid (UDCA). Or the patient may be intolerant of, or has an inadequate response to UDCA. Seladelpar or a pharmaceutically acceptable salt thereof may be administered as a monotherapy. In some embodiments, the patient may be administered a therapeutically effective amount of seladelpar lysine. Said seladelpar lysine may be seladelpar lysine dihydrate. The amount of seladelpar may be 5 mg/day or 10 mg/day.

The patient who is a poor metabolizer of CYP2C9 using moderate to strong CYP3A4 inhibitors may potentially have greater exposure to seladelpar. In some embodiments, the patient who is a poor metabolizer of CYP2C9, may be monitored or followed up for increased adverse effects if the patient is also administered the moderate to strong CYP3A4 inhibitor. The patient who is a poor metabolizer of CYP2C9 and is also administered the moderate to strong CYP3A4 inhibitor, may be monitored or followed up for increased adverse effects more frequently than a patient who is not a poor metabolizer of CYP2C9 and/or not being administered the moderate to strong CYP3A4 inhibitor if the patient is also administered the moderate to strong CYP3A4 inhibitor. The adverse effects may include headache, abdominal pain, nausea, abdominal distension, fracture, or dizziness.

The strong inhibitors may be drugs that increase the AUC of sensitive index substrates of a given metabolic pathway ≥5-fold, and the moderate inhibitors may be drugs that increase the AUC of sensitive index substrates of a given metabolic pathway ≥2- to <5-fold. In some embodiments, the moderate to strong CYP3A4 inhibitor is aprepitant, ciprofloxacin, conivaptan, crizotinib, dronedarone, fluconazole, grapefruit juice, imatinib, isavuconazole, ceritinib, cobicistat, elvitegravir, idelalisib, indinavir, lopinavir, nefazodone, nelfinavir, paritaprevir, saquinavir, telithromycin, tipranavir, voriconazole, clarithromycin, itraconazole, ketoconazole, posaconazole, erythromycin, diltiazem, ritonavir, or verapamil.

In some embodiments, the present disclosure provides a method of treating a liver disease in a patient in need thereof, wherein the patient is on concomitant bile acid binding sequestrant or bile acid sequestrant, the method comprising administering a therapeutically effective amount of seladelpar or a pharmaceutically acceptable salt thereof at an interval with administration of bile acid binding sequestrant or bile acid sequestrant. In some embodiments, the present disclosure provides a method of administering seladelpar therapy in a subject in need thereof, wherein the patient is on concomitant bile acid binding sequestrant or bile acid sequestrant, the method comprising administration of a therapeutically effective amount of seladelpar or a pharmaceutically acceptable salt thereof, at an interval with administration of bile acid binding sequestrant or bile acid sequestrant.

The liver disease may be primary biliary cholangitis (PBC). In some embodiments, the liver disease may be primary sclerosing cholangitis (PSC) or nonalcoholic steatohepatitis (NASH). Seladelpar or a pharmaceutically acceptable salt thereof may be administered orally. The patients may be also administered a therapeutically effective amount of ursodeoxycholic acid (UDCA). Or the patient may be intolerant of, or has an inadequate response to UDCA. Seladelpar or a pharmaceutically acceptable salt thereof may be administered as a monotherapy. In some embodiments, the patient may be administered a therapeutically effective amount of seladelpar lysine. Said seladelpar lysine may be seladelpar lysine dihydrate. The amount of seladelpar may be 5 mg/day or 10 mg/day.

For example, the seladelpar or a pharmaceutically acceptable salt thereof are administered sometime before or after administration of bile acid binding sequestrant or bile acid sequestrant. In some embodiments, the seladelpar or a pharmaceutically acceptable salt thereof are administered at least 1, 2, 3, 4, 5, 6, 7, 8, 9, 10, 11, or 12 hours before or after administration of bile acid binding sequestrant or bile acid sequestrant. In some embodiments, the seladelpar or a pharmaceutically acceptable salt thereof are administered at least 4 hours before or 4 hours after administration of bile acid binding sequestrant or bile acid sequestrant.

In some embodiments, the bile acid binding sequestrant or bile acid sequestrant is colesevelam, cholestyramine, or colestipol.

### Renal Impairment

Seladelpar or pharmaceutically acceptable salt thereof may be administered to patients with renal impairment. The recommended dosage in patients with mild, moderate, or severe renal impairment may be the same as in patients with normal renal function. In some embodiments, the present disclosure provides a method of treating a liver disease in a patient in need thereof, wherein the patient has renal impairment, the method comprising administering a therapeutically effective amount of seladelpar or a pharmaceutically acceptable salt thereof wherein the amount of seladelpar is 5 mg/day or 10 mg/day. In some embodiments, the present disclosure provides a method of administering seladelpar therapy in a subject in need thereof, wherein the patient has renal impairment, the method comprising administration of a therapeutically effective amount of seladelpar or a pharmaceutically acceptable salt thereof, wherein the amount of seladelpar is 5 mg/day or 10 mg/day.

The liver disease may be primary biliary cholangitis (PBC). In some embodiments, the liver disease may be primary sclerosing cholangitis (PSC) or nonalcoholic steatohepatitis (NASH). Seladelpar or a pharmaceutically acceptable salt thereof may be administered orally. The patients may be also administered a therapeutically effective amount of ursodeoxycholic acid (UDCA). Or the patient may be intolerant of, or has an inadequate response to UDCA. Seladelpar or a pharmaceutically acceptable salt thereof may be administered as a monotherapy. In some embodiments, the patient may be administered a therapeutically effective amount of seladelpar lysine. Said seladelpar lysine may be seladelpar lysine dihydrate. The amount of seladelpar may be 5 mg/day or 10 mg/day.

### Overdose

On some occasions, a patient may be overdosed with seladelpar or a pharmaceutically acceptable salt thereof. For example, overdose may happen when administered more than a recommended dosage, such as more than 5 mg/day or 10 mg/day, more than 20 mg/day, more than 50 mg/day, more than 100 mg/day, or more than 300 mg/day, or more than 2×, 3×, 5×, or 10× of the recommended dose. In some embodiments, the present disclosure provides a method of treating a liver disease in a patient in need thereof, the method comprising administering a therapeutically effective amount of seladelpar or a pharmaceutically acceptable salt thereof wherein the patient is induced emesis or performing gastric lavage if the patient is overdosed. In some embodiments, the present disclosure provides a method of administering seladelpar therapy in a subject in need thereof, the method comprising administration of a therapeutically effective amount of seladelpar or a pharmaceutically acceptable salt thereof, wherein the patient is induced emesis or performing gastric lavage if the patient is overdosed.

The liver disease may be primary biliary cholangitis (PBC). In some embodiments, the liver disease may be primary sclerosing cholangitis (PSC) or nonalcoholic steatohepatitis (NASH). Seladelpar or a pharmaceutically acceptable salt thereof may be administered orally. The patients may be also administered a therapeutically effective amount of ursodeoxycholic acid (UDCA). Or if the patient may be intolerant of, or has an inadequate response to UDCA, Seladelpar or a pharmaceutically acceptable salt thereof may be administered as a monotherapy. In some embodiments, the patient may be administered a therapeutically effective amount of seladelpar lysine. Said seladelpar lysine may be seladelpar lysine dihydrate. The administered amount of seladelpar may be 5 mg/day or 10 mg/day.

### Combination Therapies

In one embodiment, seladelpar or a pharmaceutically acceptable salt thereof may be used in combination with one or more additional therapeutic agent that are being used and/or developed to treat a liver disease, such as primary biliary cholangitis (PBC), primary sclerosing cholangitis (PSC), or nonalcoholic steatohepatitis (NASH).

In some embodiments, the one or more additional therapeutic agent may be administered with seladelpar or a pharmaceutically acceptable salt thereof. For example, a therapeutically effective amount of ursodeoxycholic acid (UDCA) may be administered with seladelpar or a pharmaceutically acceptable salt thereof. Ursodeoxycholic acid (UDCA) may be administered in an amount of 5-30 mg/kg/day. In some embodiments, UDCA may be administered in an amount of 5-25 mg/kg/day, 5-15 mg/kg/day, 10-15 mg/kg/day. In some embodiments, UDCA may be administered in an amount of 13-15 mg/kg/day.

In some embodiments, UDCA may be administered in an amount of 18-22 mg/kg/day. In some embodiments, UDCA may be administered in an amount smaller than therapeutic level (herein referred to as “subtherapeutic dose”). For example, UDCA may be administered in an amount of 10 mg/kg/day or lower, 8 mg/kg/day or lower, or 6 mg/kg/day or lower.

The patient may have inadequate response to ursodeoxycholic acid (UDCA) therapy. However, for the patient intolerant of UDCA, seladelpar or a pharmaceutically acceptable salt thereof may be administered as a monotherapy.

In some embodiments, therapeutically effective amount of one or more statins may be administered with seladelpar or a pharmaceutically acceptable salt thereof to treat liver diseases, such as PBC. In some embodiments, therapeutically effective amount of one or more statins may be administered with seladelpar or a pharmaceutically acceptable salt thereof to lower cholesterol level in patients, such as liver disease patients or PBC patients.

In some embodiments, said at least one statin is selected from atorvastatin, cerivastatin, fluvastatin, lovastatin, pitavastatin, pravastatin, rosuvastatin, simvastatin, cerivastatin, analogs thereof, and a combination thereof.

### Kits

Provided herein are also kits that include a compound of the disclosure, or a pharmaceutically acceptable salt, tautomer, stereoisomer, mixture of stereoisomers, prodrug, or deuterated analog thereof, and suitable packaging. In one embodiment, a kit further includes instructions for use. In one aspect, a kit includes a compound of the disclosure, or a pharmaceutically acceptable salt, tautomer, stereoisomer, mixture of stereoisomers, prodrug, or deuterated analog thereof, and a label and/or instructions for use of the compounds in the treatment of the indications, including the diseases or conditions, described herein.

Provided herein are also articles of manufacture that include a compound described herein or a pharmaceutically acceptable salt, tautomer, stereoisomer, mixture of stereoisomers, prodrug, or deuterated analog thereof in a suitable container. The container may be a vial, jar, ampoule, preloaded syringe, and intravenous bag.

### Dosing

The specific dose level of a compound of the present application for any particular subject will depend upon a variety of factors including the activity of the specific compound employed, the age, body weight, general health, sex, diet, time of administration, route of administration, and rate of excretion, drug combination and the severity of the particular disease in the subject undergoing therapy. For example, a dosage may be expressed as a number of milligrams of a compound described herein per kilogram of the subject's body weight (mg/kg). Dosages of between about 0.1 and 150 mg/kg may be appropriate. In some embodiments, about 0.1 and 100 mg/kg may be appropriate. In other embodiments a dosage of between 0.5 and 60 mg/kg may be appropriate. Normalizing according to the subject's body weight is particularly useful when adjusting dosages between subjects of widely disparate size, such as occurs when using the drug in both children and adult humans or when converting an effective dosage in a non-human subject such as dog to a dosage suitable for a human subject.

The daily dosage may also be described as a total amount of a compound described herein administered per dose or per day. Daily dosage of seladelpar may be between about 1 mg and 4,000 mg, between about 2,000 to 4,000 mg/day, between about 1 to 2,000 mg/day, between about 1 to 1,000 mg/day, between about 10 to 500 mg/day, between about 1 to 200 mg/day, between about 10 to 200 mg/day, between about 20 to 500 mg/day, between about 50 to 300 mg/day, between about 75 to 200 mg/day, or between about 15 to 150 mg/day. In some embodiments, the daily dosage of seladelpar may be about 10 mg/day, about 20 mg/day, about 30 mg/day, about 40 mg/day, about 50 mg/day, about 60 mg/day, about 70 mg/day, about 80 mg/day, about 90 mg/day, about 100 mg/day, about 110 mg/day, about 120 mg/day, about 130 mg/day, about 140 mg/day, about 150 mg/day, about 160 mg/day, about 170 mg/day, about 180 mg/day, about 190 mg/day, about 200 mg/day, about 250 mg/day, about 300 mg/day, or about 500 mg/day. In some embodiments, the daily dosage of seladelpar may be about 10 mg/day. In some embodiments, the daily dosage of seladelpar may be about 5 mg/day.

When administered orally, the total daily dosage for a human subject may be between 1 mg and 1,000 mg, between about 1,000-2,000 mg/day, between about 10-500 mg/day, between about 50-300 mg/day, between about 75-200 mg/day, or between about 100-150 mg/day.

The compounds of the present application or the compositions thereof may be administered once, twice, three, or four times daily, using any suitable mode described above. Also, administration or treatment with the compounds may be continued for a number of days; for example, commonly treatment would continue for at least 7 days, 14 days, or 28 days, for one cycle of treatment. Treatment cycles are well known in cancer chemotherapy, and are frequently alternated with resting periods of about 1 to 28 days, commonly about 7 days or about 14 days, between cycles. The treatment cycles, in other embodiments, may also be continuous.

In a particular embodiment, the method comprises administering to the subject an initial daily dose of about 1 to 800 mg of a compound described herein and increasing the dose by increments until clinical efficacy is achieved. Increments of about 5, 10, 25, 50, or 100 mg can be used to increase the dose. The dosage can be increased daily, every other day, twice per week, or once per week.

In some embodiments, seladelpar or pharmaceutically acceptable salt thereof may be administered in an amount equivalent to 10 mg/day of seladelpar.

### Further Embodiments

Embodiment 1. A method of treating primary biliary cholangitis (PBC) in a patient in need thereof, the method comprising administering a therapeutically effective amount of seladelpar or a pharmaceutically acceptable salt thereof to the patient, wherein administration of seladelpar or a pharmaceutically acceptable salt thereof results in one or more of: (a) an improvement in cholestasis; (b) an improvement in liver injury; and (c) an improvement or prevention of pruritus.

Embodiment 2. The method of embodiment 1, wherein the administration results in: (a) an improvement in cholestasis; (b) an improvement in liver injury; and (c) an improvement or prevention of pruritus.

Embodiment 3. The method of embodiment 1 or 2, wherein the administration results in one or more of: decrease and/or normalization in serum alkaline phosphatase (ALP) level, decrease and/or normalization in serum total bilirubin (TB) level, decrease in serum aspartate aminotransferase (AST) level, decrease in alanine aminotransferase (ALT) level, decrease in gamma-glutamyltransferase (GGT), decrease in pruritus Numerical Rating Scale (NRS), decrease in PBC-40 itch domain, and decrease in 5-D itch scale, wherein each decrease is relative to baseline.

Embodiment 4. The method of any one of embodiments 1-3, wherein the seladelpar or a pharmaceutically acceptable salt thereof is administered for up to 12 months.

Embodiment 5. The method of any one of embodiments 1-4, wherein the patient has severe pruritus (NRS≥7) at baseline.

Embodiment 6. The method of any one of embodiments 1-4, wherein the patient has moderate pruritus (NRS≥4, <7) at baseline.

Embodiment 7. The method of any one of embodiments 1-4, wherein the patient has moderate to severe pruritus (NRS≥4) at baseline.

Embodiment 8. The method of any one of embodiments 1-4, wherein the patient has no pruritus (NRS=0) at baseline or near no pruritus (NRS=0 or 1) at baseline.

Embodiment 9. The method of any one of embodiments 1-4, wherein the patient has clinically significant pruritus (PBC-40≥7) at baseline.

Embodiment 10. The method of embodiment 3, wherein the patient has moderate to severe (NRS≥4) or severe (NRS>=7) pruritus at baseline, and administration of seladelpar or a pharmaceutically acceptable salt for six months or more results in complete or near resolution of pruritus (NRS=0 or 1).

Embodiment 11. The method of embodiment 4, wherein the patient has moderate to severe (NRS≥4) or severe (NRS>=7) pruritus at baseline, and administration of seladelpar or a pharmaceutically acceptable salt for about twelve months results in complete or near resolution of pruritus (NRS=0 or 1).

Embodiment 12. The method of embodiment 3, wherein the patient has moderate to severe (NRS>=4) or severe (NRS>=7) pruritus at baseline, and administration of seladelpar or a pharmaceutically acceptable salt for six months or more results in complete resolution of pruritus (NRS=0).

Embodiment 13. The method of embodiment 4, wherein the patient has moderate to severe (NRS>=4) or severe (NRS>=7) pruritus at baseline, and administration of seladelpar or a pharmaceutically acceptable salt for about twelve months results in complete resolution of pruritus (NRS=0).

Embodiment 14. The method of embodiment 1 or 2, wherein the patient has no pruritus (NRS=0) at the baseline, and does not develop pruritus (NRS=0) after administration of seladelpar or a pharmaceutically acceptable salt for about 12 months.

Embodiment 15. The method of any one of embodiments 1-14, wherein the administration results in one or more of further results an improvement in at least one of sleep disturbance and fatigue.

Embodiment 16. The method of embodiment 15, wherein the administration results in decrease in decrease in at least one of PBC-40 sleep disturbance domain and fatigue.

Embodiment 17. The method of embodiment 16, wherein the patient has severe pruritus (NRS≥7) or clinically significant pruritus (PBC-40 itch domain ≥7).

Embodiment 18. The method of any one of embodiments 1-17, comprising administering a therapeutically effective amount of seladelpar lysine.

Embodiment 19. The method of any one of embodiments 1-18, wherein the therapeutically effective amount is equivalent to 5 mg/day or 10 mg/day of seladelpar.

Embodiment 20. The method of any one of embodiments 1-19, wherein the therapeutically effective amount is equivalent to 10 mg/day of seladelpar.

Embodiment 21. The method of any one of embodiments 1-19, wherein the therapeutically effective amount is equivalent to 5 mg/day of seladelpar.

Embodiment 22. A method of treating primary biliary cholangitis (PBC) in a patient in need thereof, the method comprising administering seladelpar or a pharmaceutically acceptable salt thereof to the patient in an amount equivalent to 5 mg/day or 10 mg/day of seladelpar, wherein administration of seladelpar or a pharmaceutically acceptable salt thereof results in one or more of: (a) an improvement in cholestasis; (b) an improvement in liver injury; and (c) an improvement or prevention of pruritus.

Embodiment 23. The method of embodiment 22, wherein the administration results in: (a) an improvement in cholestasis; (b) an improvement in liver injury; and (c) an improvement or prevention of pruritus.

Embodiment 24. The method of embodiment 22 or 23, wherein the administration results in one or more of: decrease and/or normalization in serum alkaline phosphatase (ALP) level, decrease and/or normalization in serum total bilirubin (TB) level, decrease in serum aspartate aminotransferase (AST) level, decrease in alanine aminotransferase (ALT) level, decrease in gamma-glutamyltransferase (GGT), decrease in pruritus Numerical Rating Scale (NRS), decrease in PBC-40 itch domain, and decrease in 5-D itch scale, wherein each decrease is relative to baseline.

Embodiment 25. The method of any one of embodiments 22-24, wherein the seladelpar or a pharmaceutically acceptable salt thereof is administered for up to 12 months.

Embodiment 26. The method of any one of embodiments 22-25, comprising administering 14.1 mg/day of seladelpar lysine dihydrate.

Embodiment 27. A method of treating a liver disease in a patient in need thereof, comprising administering a therapeutically effective amount of seladelpar or a pharmaceutically acceptable salt thereof to the patient, wherein the patient does not have decompensated cirrhosis.

Embodiment 28. The method of embodiment 27, wherein the liver disease is primary biliary cholangitis (PBC).

Embodiment 29. The method of embodiment 27 or 28, wherein seladelpar or a pharmaceutically acceptable salt thereof is administered orally.

Embodiment 30. The method of any one of embodiments 27 to 29, wherein the patient is intolerant of, or has an inadequate response to ursodeoxycholic acid (UDCA) therapy.

Embodiment 31. The method of embodiment 30, wherein the patient has serum alkaline phosphatase (ALP) level at or above 1.5 times or higher of a normal level after the ursodeoxycholic acid (UDCA) therapy.

Embodiment 32. The method of embodiment 31, wherein the patient has serum alkaline phosphatase (ALP) level at or above 1.67 times or higher of a normal level after the ursodeoxycholic acid (UDCA) therapy.

Embodiment 33. The method of any one of embodiments 27 to 32, wherein seladelpar or a pharmaceutically acceptable salt thereof is administered as a monotherapy.

Embodiment 34. The method of any one of embodiments 27 to 32, further comprising administering a therapeutically effective amount of ursodeoxycholic acid (UDCA).

Embodiment 35. The method of embodiment 34, wherein ursodeoxycholic acid (UDCA) is administered in an amount of 5-30 mg/kg/day.

Embodiment 36. The method of embodiment 34 or 35, wherein ursodeoxycholic acid (UDCA) is administered in an amount of 13-15 mg/kg/day.

Embodiment 37. The method of embodiment 34, wherein ursodeoxycholic acid (UDCA) is administered in an amount of 18-22 mg/kg/day.

Embodiment 38. The method of embodiment 34, wherein ursodeoxycholic acid (UDCA) is administered in an amount of 8 mg/kg/day or lower.

Embodiment 39. The method of any one of embodiments 27 to 38, comprising administering a therapeutically effective amount of seladelpar lysine.

Embodiment 40. The method of any one of embodiments 27 to 39, wherein the amount of seladelpar is 5 mg/day or 10 mg/day.

Embodiment 41. The method of any one of embodiments 27 to 40, further comprising monitoring for evidence of decompensation if the patient has cirrhosis.

Embodiment 42. A method of administering seladelpar therapy in a subject in need thereof, comprising administration of a therapeutically effective amount of seladelpar or a pharmaceutically acceptable salt thereof to a patient, wherein the patient does not have decompensated cirrhosis.

Embodiment 43. The method of embodiment 42, wherein seladelpar or a pharmaceutically acceptable salt thereof is seladelpar lysine.

Embodiment 44. The method of embodiment 42 or 43, wherein the amount of seladelpar is 5 mg/day or 10 mg/day.

Embodiment 45. A method of treating a liver disease in a patient in need thereof, comprising administering a therapeutically effective amount of seladelpar or a pharmaceutically acceptable salt thereof to the patient and monitoring or testing the patient for signs of drug-induced liver injury or abnormalities.

Embodiment 46. The method of embodiment 45, wherein the liver disease is primary biliary cholangitis (PBC).

Embodiment 47. The method of any one of embodiments 45 or 46, comprising administering a therapeutically effective amount of seladelpar lysine.

Embodiment 48. The method of any one of embodiments 45 to 47, wherein the amount of seladelpar is 5 mg/day or 10 mg/day.

Embodiment 49. The method of any one of embodiments 45 to 48, comprising monitoring one or more of ALT, AST, total bilirubin, ALP, jaundice, fatigue, nausea, right upper quadrant pain or tenderness, fever, rash, and eosinophilia.

Embodiment 50. The method of any of embodiments 45 to 49, comprising monitoring for signs of muscle injury or abnormalities.

Embodiment 51. The method of any of embodiments 45 to 50, comprising monitoring creatine phosphokinase, muscle pain, or muscle tenderness.

Embodiment 52. A method of administering seladelpar therapy in a subject in need thereof, comprising administration of a therapeutically effective amount of seladelpar or a pharmaceutically acceptable salt thereof to a patient and monitoring or testing the patient for signs of drug-induced liver injury or abnormalities.

Embodiment 53. The method of embodiment 52, wherein the amount of seladelpar is 5 mg/day or 10 mg/day.

Embodiment 54. The method of embodiment 52 or 53, wherein signs of drug-induced liver injury or abnormalities comprises one or more of ALT, AST, total bilirubin, ALP, jaundice, fatigue, nausea, right upper quadrant pain or tenderness, fever, rash, and eosinophilia.

Embodiment 55. A method of treating a liver disease in a patient in need thereof, comprising administering a therapeutically effective amount of seladelpar or a pharmaceutically acceptable salt thereof to the patient and testing and optionally treating the patient for bone density and/or bone health.

Embodiment 56. The method of embodiment 55, wherein the liver disease is primary biliary cholangitis (PBC).

Embodiment 57. The method of embodiment 55 or 56, wherein seladelpar or a pharmaceutically acceptable salt thereof is administered orally.

Embodiment 58. The method of any one of embodiments 55 to 57, wherein the patient is intolerant of, or has an inadequate response to ursodeoxycholic acid (UDCA).

Embodiment 59. The method of any one of embodiments 55 to 58, wherein seladelpar or a pharmaceutically acceptable salt thereof is administered as a monotherapy.

Embodiment 60. The method of any one of embodiments 55 to 58, further comprising administering a therapeutically effective amount of ursodeoxycholic acid (UDCA).

Embodiment 61. The method of embodiment 60, wherein ursodeoxycholic acid (UDCA) is administered in an amount of 13-15 mg/kg/day.

Embodiment 62. The method of embodiments 60, wherein ursodeoxycholic acid (UDCA) is administered at subtherapeutic dose.

Embodiment 63. A method of providing seladelpar therapy to a patient in need thereof, comprising administration of a therapeutically effective amount of seladelpar, further comprising monitoring and optionally treating the patient for bone density and/or bone health.

Embodiment 64. The method of embodiment 63, comprising administering a therapeutically effective amount of seladelpar lysine.

Embodiment 65. The method of embodiment 63 or 64, wherein the amount of seladelpar is 5 mg/day or 10 mg/day.

Embodiment 66. The method of embodiment 65, wherein the amount of seladelpar is 5 mg/day.

Embodiment 67. The method of embodiment 65, wherein the amount of seladelpar is 10 mg/day.

Embodiment 68. A method of treating a liver disease in a patient in need thereof, comprising administering a therapeutically effective amount of seladelpar or a pharmaceutically acceptable salt thereof to the patient, wherein the patient does not have complete biliary obstruction.

Embodiment 69. The method of embodiment 68, wherein the liver disease is primary biliary cholangitis (PBC).

Embodiment 70. The method of embodiment 68 or 69, wherein seladelpar or a pharmaceutically acceptable salt thereof is administered orally.

Embodiment 71. The method of any one of embodiment 68 to 70, wherein the patient is intolerant of, or has an inadequate response to ursodeoxycholic acid (UDCA).

Embodiment 72. The method of any one of embodiments 68 to 71, wherein seladelpar or a pharmaceutically acceptable salt thereof is administered as a monotherapy.

Embodiment 73. The method of any one of embodiments 68 to 71, further comprising administering a therapeutically effective amount of ursodeoxycholic acid (UDCA).

Embodiment 74. The method of any one of embodiments 68 to 73, comprising administering a therapeutically effective amount of seladelpar lysine.

Embodiment 75. The method of any one of embodiments 68 to 74, wherein the amount of seladelpar is 5 mg/day or 10 mg/day.

Embodiment 76. The method of any one of embodiments 68 to 75, wherein the amount of seladelpar is 5 mg/day.

Embodiment 77. The method of any one of embodiments 68 to 75, wherein the amount of seladelpar is 10 mg/day.

Embodiment 78. The method of any one of embodiments 68 to 77, further comprising testing the patient for biliary obstruction.

Embodiment 79. The method of embodiment 78, further comprising stopping the administration of seladelpar or a pharmaceutically acceptable salt thereof if biliary obstruction is suspected.

Embodiment 80. A method of providing seladelpar therapy to a patient in need thereof, comprising administering a therapeutically effective amount of seladelpar or a salt thereof, wherein the patient is not identified to have complete biliary obstruction.

Embodiment 81. The method of embodiment 80, comprising administering a therapeutically effective amount of seladelpar lysine.

Embodiment 82. The method of embodiment 80 or 81, wherein the amount of seladelpar is 5 mg/day or 10 mg/day.

Embodiment 83. The method of claim 82, wherein the amount of seladelpar is 10 mg/day.

Embodiment 84. The method of any one of embodiments 81 to 83, further comprising testing the patient for biliary obstruction.

Embodiment 85. The method of embodiment 81, further comprising stopping the administration of seladelpar or a pharmaceutically acceptable salt thereof if biliary obstruction is suspected.

Embodiment 86. A method of treating a liver disease in a patient in need thereof, wherein the patient has renal impairment, the method comprising administering a therapeutically effective amount of seladelpar or a pharmaceutically acceptable salt thereof to the patient, wherein the amount of seladelpar is 5 mg/day or 10 mg/day.

Embodiment 87. The method of embodiment 86, wherein the liver disease is primary biliary cholangitis (PBC).

Embodiment 88. The method of embodiment 86 or 87, wherein seladelpar or a pharmaceutically acceptable salt thereof is administered orally.

Embodiment 89. The method of any one of embodiments 86 to 88, wherein the patient is intolerant of, or has an inadequate response to ursodeoxycholic acid (UDCA).

Embodiment 90. The method of any one of embodiments 86 to 89, wherein seladelpar or a pharmaceutically acceptable salt thereof is administered as a monotherapy.

Embodiment 91. The method of any one of embodiments 86 to 89, further comprising administering a therapeutically effective amount of ursodeoxycholic acid (UDCA).

Embodiment 92. The method of embodiment 91, wherein UDCA is administered in an amount of 13-15 mg/kg/day.

Embodiment 93. The method of any one of embodiments 86 to 92, comprising administering a therapeutically effective amount of seladelpar lysine.

Embodiment 94. The method of any one of embodiments 86 to 93, wherein the renal impairment is mild, moderate, or severe renal impairment.

Embodiment 95. A method of administering seladelpar therapy in a patient in need thereof, wherein the patient has renal impairment, the method comprising administering a therapeutically effective amount of seladelpar or a pharmaceutically acceptable salt thereof, wherein the amount of seladelpar is 5 mg/day or 10 mg/day.

Embodiment 96. The method of embodiment 95, comprising administering a therapeutically effective amount of seladelpar lysine.

Embodiment 97. The method of embodiment 95 or 96, wherein the renal impairment is mild, moderate, or severe renal impairment.

Embodiment 98. A pharmaceutical composition comprising: seladelpar or a pharmaceutically acceptable salt thereof in an amount equivalent to 5 mg/day or 10 mg of seladelpar, butylated hydroxytoluene, colloidal silicon dioxide, croscarmellose sodium, magnesium stearate, mannitol, and microcrystalline cellulose.

Embodiment 99. The pharmaceutical composition of embodiment 98, comprising 14.1 mg of seladelpar lysine dihydrate.

Embodiment 100. The pharmaceutical composition of embodiment 98 or 99, wherein each of colloidal silicon dioxide, croscarmellose sodium, magnesium stearate, mannitol, and microcrystalline cellulose are substantially free of peroxides.

Embodiment 101. The pharmaceutical composition of embodiment 98 or 99, wherein the composition is substantially free of peroxides.

Embodiment 102. The pharmaceutical composition of any one of embodiments 98-101, comprising granules.

Embodiment 103. The pharmaceutical composition of any one of embodiments 98-101, wherein the composition is made by roller compaction and/or dry granulation.

Embodiment 104. A pharmaceutical dosage form comprising the pharmaceutical composition of any one of embodiments 98-103 in a capsule.

Embodiment 105. A method of increasing serum level of one or more metabolism markers metabolism markers in a patient in need thereof, the method comprising administering a therapeutically effective amount of seladelpar or a pharmaceutically acceptable salt thereof to the patient, wherein said one or more metabolism markers are selected from the group consisting of carnitine, acetylcarnitine, acylcarnitines, N,N,N-trimethyl-5-aminovalerate, and branched-chain amino acid catabolites.

Embodiment 106. A method of decreasing serum level of one or more metabolism markers in a patient in need thereof, the method comprising administering a therapeutically effective amount of seladelpar or a pharmaceutically acceptable salt thereof to the patient, wherein said one or more metabolism markers are selected from dicarboxylates, saturated and unsaturated fatty acids, and lipid-derived inflammatory mediators.

Embodiment 107. A method of decreasing serum level of at least one cholesterol level in a patient in need thereof, the method comprising administering a therapeutically effective amount of seladelpar or a pharmaceutically acceptable salt thereof to the patient, wherein said cholesterol level is total cholesterol level, low-density lipoprotein (LDL) level, or triglyceride level.

Embodiment 108. The method of embodiment 107, wherein the method further comprises administering at least one statin to the patient, wherein the administration does not cause significant adverse effects.

Embodiment 109. The method of embodiment 107, wherein said at least one statin is selected from atorvastatin, cerivastatin, fluvastatin, lovastatin, pitavastatin, pravastatin, rosuvastatin, simvastatin, cerivastatin, analogs thereof, and a combination thereof.

Embodiment 110. A method of treating primary biliary cholangitis (PBC) in a patient in need thereof, the method comprising administering a therapeutically effective amount of seladelpar or a pharmaceutically acceptable salt thereof and a therapeutically effective amount of one or more statins, to the patient.

Embodiment 111. The method of embodiment 110, wherein said at least one statin is selected from atorvastatin, simvastatin, rospivastatin, gravistatin, and lovastatin.

Embodiment 112. The method of embodiment 110 or 111, wherein the therapeutically effective amount for seladelpar a pharmaceutically acceptable salt thereof or is equivalent to 5 mg/day or 10 mg/day of seladelpar.

Embodiment 113. The method of embodiment 110 or 111, wherein the administering results in reduction of at least one of total cholesterol level, low-density lipoprotein (LDL) level, and triglyceride level.

Embodiment 114. The method of any one of embodiments 110-113, wherein the administering is for up to 12 months.

Embodiment 115. A method of increasing expression level of one or more genes selected from OCTN2, CPT1A, CPT2, CACT, and CRAT, comprising administering a therapeutically effective amount of seladelpar or a pharmaceutically acceptable salt thereof.

Embodiment 116. A method of determining activities of seladelpar in a subject, comprising measuring the serum level of one or more of metabolites selected from the group consisting of: carnitine, acetylcarnitine, other acylcarnitines (i.e., short-, medium- and long-chain acylcarnitines), N,N,N-trimethyl-5-aminovalerate (TMAVA), branched-chain amino acid catabolites, dicarboxylates, saturated and unsaturated fatty acids, and lipid-derived inflammatory mediators.

Embodiment 117. A method of determining activities of seladelpar in a subject, comprising measuring the serum level of the expression level of one or more genes selected from OCTN2, CPT1A, CPT2, CACT, and CRAT in the subject.

Embodiment 118. A method of treating primary biliary cholangitis (PBC) in a patient in need thereof, comprising administering a therapeutically effective amount of seladelpar or a pharmaceutically acceptable salt thereof to a subject, and measuring (a) the serum level of one or more of metabolites selected from the group consisting of: carnitine, acetylcarnitine, other acylcarnitines (i.e., short-, medium- and long-chain acylcarnitines), N,N,N-trimethyl-5-aminovalerate (TMAVA), branched-chain amino acid catabolites, dicarboxylates, saturated and unsaturated fatty acids, and lipid-derived inflammatory mediators; and/or (b) the expression level of one or more genes selected from OCTN2, CPT1A, CPT2, CACT, and CRAT in the subject.

Embodiment 119. The method of embodiments 1-4, 22-26, 45-97, wherein:
(a) the patient is less than 65 years old, or 65 years old or older;
(b) the patient was less than 50 years old at PBC diagnosis, or 50 years old or older;
(c) the patient is white, Asian, black, Latino, or non-Latino;
(d) the patient is male or female;
(e) the patient is from North America, Europe, or rest-of-world;
(f) the patient has serum ALP level of <350 U/L or ≥350 U/L at baseline;
(g) the patient has Total Bilirubin (TB) level of <0.6×ULN or ≥0.6×ULN at baseline;
(h) the patient has Total Bilirubin (TB) level of <1×ULN or ≥1×ULN at baseline;
(i) the patient has pruritus NRS of <4 or ≥4 at baseline; or
(j) the patient has cirrhosis or does not have cirrhosis.

Embodiment 120. A method of treating primary biliary cholangitis (PBC) in a patient in need thereof, the method comprising administering a therapeutically effective amount of seladelpar or a pharmaceutically acceptable salt thereof to the patient, wherein:
(a) the patient is less than 65 years old, or 65 years old or older;
(b) the patient was less than 50 years old at PBC diagnosis, or 50 years old or older;
(c) the patient is white, Asian, black, Latino, or non-Latino;
(d) the patient is male or female;
(e) the patient is from North America, Europe, or rest-of-world;
(f) the patient has serum ALP level of <350 U/L or ≥350 U/L at baseline;
(g) the patient has Total Bilirubin (TB) level of <0.6×ULN or ≥0.6×ULN at baseline;
(h) the patient has Total Bilirubin (TB) level of <1×ULN or ≥1×ULN at baseline;
(i) the patient has pruritus NRS of <4 or ≥4 at baseline; or
(j) the patient has cirrhosis or does not have cirrhosis.

Embodiment 121: A method of treating a liver disease in a patient in need thereof, comprising administering a therapeutically effective amount of seladelpar or a pharmaceutically acceptable salt thereof, while avoiding co-administration of an OAT3 inhibitor or a strong CYP2C9 inhibitor.

Embodiment 122: The method of embodiment 121, wherein the liver disease is primary biliary cholangitis (PBC).

Embodiment 123: The method of embodiment 121 or 122, wherein seladelpar or a pharmaceutically acceptable salt thereof is administered orally.

Embodiment 124: The method of any one of embodiments 121-123, further comprising administering a therapeutically effective amount of ursodeoxycholic acid (UDCA).

Embodiment 125: The method of any one of embodiments 121-124, wherein the patient is intolerant of, or has an inadequate response to UDCA.

Embodiment 126: The method of any one of embodiments 121-125, wherein seladelpar or a pharmaceutically acceptable salt thereof is administered as a monotherapy.

Embodiment 127: The method of any one of embodiments 121-126, comprising administering the therapeutically effective amount of seladelpar lysine.

Embodiment 128: The method of any one of embodiments 121-127, wherein the amount of seladelpar is 10 mg/day.

Embodiment 129: The method of any one of embodiments 121-128, further comprising discontinuing administration of an OAT3 inhibitor or a strong CYP2CP inhibitor prior to the administration of seladelpar or a pharmaceutically acceptable salt thereof.

Embodiment 130: The method of any one of embodiments 121-129, wherein the OAT3 inhibitor is probenecid.

Embodiment 131: The method of any one of embodiments 121-130, wherein the strong CYP2C9 inhibitor is sulphaphenazole.

Embodiment 132: A method of administering seladelpar therapy to a patient in need thereof, comprising administering to the patient a therapeutically effective amount of seladelpar while avoiding co-administration of an OAT3 inhibitor or a strong CYP2C9 inhibitor.

Embodiment 133: The method of embodiment 132, comprising administering the therapeutically effective amount of seladelpar lysine.

Embodiment 134: The method of embodiment 132 or 133, wherein the amount of seladelpar is 10 mg/day.

Embodiment 15: The method of any one of embodiments 132-134, further comprising discontinuing administration of an OAT3 inhibitor or a strong CYP2CP inhibitor prior to the administration of seladelpar or a pharmaceutically acceptable salt thereof.

Embodiment 136: The method of any one of embodiments 132-135, wherein the OAT3 inhibitor is probenecid.

Embodiment 137: The method of any one of embodiments 132-136, wherein the strong CYP2C9 inhibitor is sulphaphenazole.

Embodiment 138: A method of treating a liver disease in a patient in need thereof, comprising administering a therapeutically effective amount of seladelpar or a pharmaceutically acceptable salt thereof and advising the patient that co-administering with rifampin may result in delayed or suboptimal biochemical response of the seladelpar therapy.

Embodiment 139: The method of embodiment 138, wherein the liver disease is primary biliary cholangitis (PBC).

Embodiment 140: The method of embodiment 138 or 139, wherein seladelpar or a pharmaceutically acceptable salt thereof is administered orally.

Embodiment 141: The method of any one of embodiments 138 to 140, further comprising administering a therapeutically effective amount of ursodeoxycholic acid (UDCA).

Embodiment 142: The method of embodiment 138, wherein the patient is intolerant of, or has an inadequate response to UDCA.

Embodiment 143: The method of any one of embodiments 138 to 140, wherein seladelpar or a pharmaceutically acceptable salt thereof is administered as a monotherapy.

Embodiment 144: The method of any one of embodiments 138 to 143, comprising administering the therapeutically effective amount of seladelpar lysine.

Embodiment 145: The method of any one of embodiments 138 to 144, wherein the amount of seladelpar is 10 mg/day.

Embodiment 146: The method of any one of embodiments 138 to 145, further comprising monitoring and/or testing for delayed or suboptimal biochemical response of the seladelpar therapy if the patient is co-administered with rifampin.

Embodiment 147: A method of administering seladelpar therapy to a patient in need thereof, comprising administering to the patient a therapeutically effective amount of seladelpar and advising the patient that co-administering with rifampin may result in delayed or suboptimal biochemical response of the seladelpar therapy.

Embodiment 148: The method of embodiment 147, comprising administering the therapeutically effective amount of seladelpar lysine.

Embodiment 149: The method of embodiment 147 or 148, wherein the amount of seladelpar is 10 mg/day.

Embodiment 150: The method of any one of embodiments 147 to 149, further comprising monitoring and/or testing for delayed or suboptimal biochemical response of the seladelpar therapy if the patient is co-administered with rifampin.

Embodiment 151: A method of treating a liver disease in a patient in need thereof, comprising administering a therapeutically effective amount of seladelpar or a pharmaceutically acceptable salt thereof and advising the patient that co-administering with a dual inhibitor of CYP2C9 and CYP3A4 or a BCRP inhibitor may result in adverse effects.

Embodiment 152: The method of embodiment 151, wherein the liver disease is primary biliary cholangitis (PBC).

Embodiment 153: The method of embodiment 151 or 152, wherein seladelpar or a pharmaceutically acceptable salt thereof is administered orally.

Embodiment 154: The method of any one of embodiments 151 to 153, further comprising administering a therapeutically effective amount of ursodeoxycholic acid (UDCA).

Embodiment 155: The method of embodiment 154, wherein the patient is intolerant of, or has an inadequate response to UDCA.

Embodiment 156: The method of any one of embodiments 151 to 153, wherein seladelpar or a pharmaceutically acceptable salt thereof is administered as a monotherapy.

Embodiment 157: The method of any one of embodiments 151 to 156, comprising administering the therapeutically effective amount of seladelpar lysine.

Embodiment 158: The method of any one of embodiments 151 to 157, wherein the amount of seladelpar is 10 mg/day.

Embodiment 159: The method of any one of embodiments 151 to 158, further comprising monitoring and/or testing for adverse effects.

Embodiment 160: The method of any one of embodiments 151 to 159, wherein the adverse effects is headache, abdominal pain, nausea, abdominal distension, fracture, or dizziness.

Embodiment 161: The method of any one of embodiments 151 to 160, wherein the dual inhibitor of CYP2C9 and CYP3A4 is fluconazole or amiodarone.

Embodiment 162: The method of any one of embodiments 151 to 161, wherein the BCRP inhibitor is cyclosporine.

Embodiment 163: A method of administering seladelpar therapy to a patient in need thereof, comprising administering to the patient a therapeutically effective amount of seladelpar, advising the patient that co-administering with a dual inhibitor of CYP2C9 and CYP3A4 may result in adverse effects.

Embodiment 164: The method of embodiment 163, comprising administering the therapeutically effective amount of seladelpar lysine.

Embodiment 165: The method of embodiment 163 or 164, wherein the amount of seladelpar is 10 mg/day.

Embodiment 166: The method of any one of embodiments 163 to 165, further comprising monitoring and/or testing for adverse effects.

Embodiment 167: The method of any one of embodiments 163 to 166, wherein the adverse effects is headache, abdominal pain, nausea, abdominal distension, fracture, or dizziness.

Embodiment 168: The method of any one of embodiments 163 to 167, wherein the dual inhibitor of CYP2C9 and CYP3A4 is fluconazole or amiodarone.

Embodiment 169: The method of any one of embodiments 163 to 168, wherein the BCRP inhibitor is cyclosporine.

Embodiment 170: A method of treating a liver disease in a patient in need thereof, wherein the patient is a poor metabolizer of CYP2C9, the method comprising administering a therapeutically effective amount of seladelpar or a pharmaceutically acceptable salt thereof and advising the patient that co-administering with a moderate to strong CYP3A4 inhibitor may result in increased exposure of seladelpar.

Embodiment 171: The method of embodiment 170, wherein the liver disease is primary biliary cholangitis (PBC).

Embodiment 172: The method of embodiment 170 or 171, wherein seladelpar or a pharmaceutically acceptable salt thereof is administered orally.

Embodiment 173: The method of any one of embodiments 170 to 172, further comprising administering a therapeutically effective amount of ursodeoxycholic acid (UDCA).

Embodiment 174: The method of embodiment 173, wherein the patient is intolerant of, or has an inadequate response to UDCA.

Embodiment 175: The method of any one of embodiments 170 to 173, wherein seladelpar or a pharmaceutically acceptable salt thereof is administered as a monotherapy.

Embodiment 176: The method of any one of embodiments 170 to 174, comprising administering the therapeutically effective amount of seladelpar lysine.

Embodiment 177: The method of any one of embodiments 170 to 175, wherein the amount of seladelpar is 10 mg/day.

Embodiment 178: The method of any one of embodiments 170 to 177, further comprising monitoring and/or testing for increased adverse effects if the patient is also administered the moderate to strong CYP3A4 inhibitor.

Embodiment 179: A method of administering seladelpar therapy to a patient in need thereof, wherein the patient is a poor metabolizer of CYP2C9, the method comprising administering to the patient a therapeutically effective amount of seladelpar, advising the patient that co-administering with a moderate to strong CYP3A4 inhibitor may result in increased exposure of seladelpar.

Embodiment 180: The method of embodiment 179, comprising administering the therapeutically effective amount of seladelpar lysine.

Embodiment 181: The method of embodiment 179 or 180, wherein the amount of seladelpar is 10 mg/day.

Embodiment 182: The method of any one of embodiments 179 to 181, further comprising monitoring and/or testing for increased adverse effects if the patient is also administered the moderate to strong CYP3A4 inhibitor.

Embodiment 183: A method of treating a liver disease in a patient in need thereof, wherein the patient is on concomitant bile acid binding sequestrant or bile acid sequestrant, the method comprising administering a therapeutically effective amount of seladelpar or a pharmaceutically acceptable salt thereof at least 4 hours before or 4 hours after administration of bile acid binding sequestrant or bile acid sequestrant.

Embodiment 184: The method of embodiment 183, wherein the liver disease is primary biliary cholangitis (PBC).

Embodiment 185: The method of embodiment 183 or 184, wherein seladelpar or a pharmaceutically acceptable salt thereof is administered orally.

Embodiment 186: The method of any one of embodiments 183 to 185, further comprising administering a therapeutically effective amount of ursodeoxycholic acid (UDCA).

Embodiment 187: The method of embodiment 186, wherein the patient is intolerant of, or has an inadequate response to UDCA.

Embodiment 188: The method of any one of embodiments 183 to 185, wherein seladelpar or a pharmaceutically acceptable salt thereof is administered as a monotherapy.

Embodiment 189: The method of any one of embodiments 183 to 188, comprising administering the therapeutically effective amount of seladelpar lysine.

Embodiment 190: The method of any one of embodiments 183 to 189, wherein the amount of seladelpar is 10 mg/day.

Embodiment 191: A method of administering seladelpar therapy in a patient in need thereof, wherein the patient is on concomitant bile acid binding sequestrant or bile acid sequestrant, the method comprising administering a therapeutically effective amount of seladelpar or a pharmaceutically acceptable salt thereof at least 4 hours before or 4 hours after administration of bile acid binding sequestrant or bile acid sequestrant.

Embodiment 192: The method of embodiment 191, comprising administering the therapeutically effective amount of seladelpar lysine.

Embodiment 193: The method of embodiment 191 or 192, wherein the amount of seladelpar is 10 mg/day.

Embodiment 194. A method of treating a liver disease in a patient in need thereof, comprising administering a therapeutically effective amount of seladelpar or a pharmaceutically acceptable salt thereof, while avoiding co-administration of probenecid.

Embodiment 195: A method of administering seladelpar therapy to a patient in need thereof, comprising administering to the patient a therapeutically effective amount of seladelpar while avoiding co-administration of probenecid.

Embodiment 196. A method of treating a liver disease in a patient in need thereof, comprising administering a therapeutically effective amount of seladelpar or a pharmaceutically acceptable salt thereof, and advising the patient that co-administering with a strong CYP2C9 inhibitor may result in adverse effects.

Embodiment 197: A method of administering seladelpar therapy to a patient in need thereof, comprising administering to the patient a therapeutically effective amount of seladelpar, and advising the patient that co-administering with a strong CYP2C9 inhibitor may result in adverse effects.

Embodiment 198: A method of treating a liver disease in a patient in need thereof, comprising administering a therapeutically effective amount of seladelpar or a pharmaceutically acceptable salt thereof and advising the patient that co-administering with cyclosporine may result in adverse effects.

Embodiment 199: A method of administering seladelpar therapy to a patient in need thereof, comprising administering a therapeutically effective amount of seladelpar or a pharmaceutically acceptable salt thereof and advising the patient that co-administering with cyclosporine may result in adverse effects.

Embodiment 200: A method of treating a liver disease in a patient in need thereof, comprising administering a therapeutically effective amount of seladelpar or a pharmaceutically acceptable salt thereof to the patient, wherein the patient is induced emesis or performing gastric lavage if the patient is overdosed.

Embodiment 201. A method of treating patient on seladelpar therapy for overdose of seladelpar, comprising inducing emesis or performing gastric lavage.

### EXAMPLES

The following examples are included to demonstrate specific embodiments of the disclosure. It should be appreciated by those of skill in the art that the techniques disclosed in the examples which follow represent techniques to function well in the practice of the disclosure, and thus can be considered to constitute specific modes for its practice. However, those of skill in the art should, in light of the present disclosure, appreciate that many changes can be made in the specific embodiments which are disclosed and still obtain a like or similar result without departing from the spirit and scope of the disclosure.

### Example 1: Pharmacokinetics

The effect of a single oral dose of up to 360 mg of seladelpar in 9 healthy male subjects were obtained. After oral administration of seladelpar, plasma concentration of seladelpar and metabolites were determined using a LC-MS/MS method. Following a single dose administration, seladelpar systemic exposure increased dose-proportionally from 2 mg to 15 mg (1.5 times the recommended dosage) and greater than dose proportionally at higher doses.

In a separate study, the effect of a single oral dose of 10 or 200 mg of seladelpar was compared to placebo in 12 healthy male and female subjects. After oral administration of 10 or 200 mg, plasma concentration of seladelpar and metabolites were determined using a LC-MS/MS method. For a dose increase of 20-fold from 10 mg to 200 mg, mean Cₘₐₓ and mean AUC for seladelpar increased 70-fold and 27-fold, respectively.

In an open-label, single sequence study, a single oral dose of 10 mg seladelpar was administered to healthy subjects (4 males, 4 females), and blood, plasma, urine and feces sample were collected at intervals through day 5. In a single-center double-blind, randomized sequential group, placebo-controlled study, seladelpar (50, 100, or 200 mg) or placebo was administered once per day to 12 healthy male subjects. Following once daily dosing, seladelpar steady-state was achieved by day 4 and AUC increase was less than 30%.

In studies for PBC patients, mean (SD) Cₘₐₓ and AUC for seladelpar was 103 (29.3) ng/mL and was 902 (238) ng-h/mL, respectively at steady state following once daily dosing of 10 mg.

### Absorption

In an open-label, single sequence study of the absorption, metabolism and excretion of seladelpar in healthy subjects (4 males and 4 females), when 10 mg seladelpar single dose was administered, a median time to peak concentration (Tₘₐₓ) was 1.5 hours for seladelpar.

In another open-label, randomized food effect study, no clinically significant differences in seladelpar pharmacokinetics were observed following co-administration of a high-fat meal in healthy subjects.

### Distribution

From multiple clinical studies, seladelpar steady state apparent volume of distribution was approximately 110.3 L. Seladelpar plasma protein binding was greater than 99%.

### Elimination

From multiple clinical studies, for PBC patients, the apparent oral clearance of seladelpar is 12.6 L/h. Following administration of a single dose of 10 mg seladelpar in healthy subjects, mean elimination half-life was 6 hours for seladelpar. In PBC patients, the half-life range was 3.8 to 6.7 hours for seladelpar.

Seladelpar is primarily metabolized in vitro by CYP2C9 and to a lesser extent by CYP2C8 and CYP3A4 resulting in the three major metabolites: seladelpar sulfoxide (M1), desethyl-seladelpar (M2), and desethyl-seladelpar sulfoxide (M3). In an open-label, single sequence study of the absorption, metabolism and excretion of seladelpar in healthy subjects (4 males and 4 females), after 10 mg seladelpar single dose was administered, the metabolite-to-parent AUC ratios were 0.36, 2.32 and 0.63 for M1, M2 and M3, respectively.

Seladelpar was primarily eliminated in urine as metabolites. In an open-label, single sequence study of the absorption, metabolism and excretion of seladelpar in healthy subjects (4 males and 4 females), following a single oral dose of 10 mg radiolabeled seladelpar in humans, approximately 73.4% of the dose was recovered in urine (less than 0.01% unchanged) and 19.5% in feces (2.02% unchanged) within 216 hours.

From multiple clinical studies, no clinically significant differences in the pharmacokinetics of seladelpar were observed based on age (19 to 79 years old), body mass index (BMI) (17.6 to 45.0 kg/m²), weight (45.8 to 127.5 kg), sex, and race (white, black, or other).

### Example 2: Specific Populations

No clinically significant differences in the pharmacokinetics of seladelpar were observed based on age (19 to 79 years old), body mass index (BMI) (17.6 to 45.0 kg/m2), weight (45.8 to 127.5 kg), sex, and race (white, black, or other).

Patients with Renal Impairment

An open-label, non-randomized, parallel-group study was conducted to determine the effect of renal impairment on the PK, safety, and tolerability of a single oral dose of seladelpar compared with matched healthy subjects with normal renal function. Subjects received a single oral dose of 10 mg seladelpar (capsule) administered orally on Day 1 after a fast of at least 10 hours. Plasma and urine concentrations of seladelpar and metabolites were determined using validated LC-MS/MS methods.

In subjects with mild (eGFR≥60 to <90 mL/min/1.73 m², MDRD), moderate (eGFR≥30 to <60 mL/min/1.73 m²), and severe (<30 mL/min/1.73 m² and not on dialysis) renal impairment, the AUC_{inf} of seladelpar was 10% higher, 54% higher, and similar to that in subjects with normal renal function, respectively, after administration of a single 10 mg dose of seladelpar. The difference in Cₘₐₓ of seladelpar was less than 18% in subjects with renal impairment compared to subjects with normal renal function. The pharmacokinetics of seladelpar have not been studied in patients requiring hemodialysis.

Patients with Hepatic Impairment

A study to evaluate the PK parameters, safety, and tolerability of a single oral dose of 10 mg seladelpar in subjects with varying degrees of hepatic function was conducted. Subjects were enrolled into 4 cohorts (8 subjects per cohort) according to hepatic function status (normal function and mild, moderate, and severe impairment) classified by Child-Pugh score. Subjects received a single oral dose of 10 mg seladelpar on Day 1 after an overnight (≥8 hour) fast. Plasma concentrations of seladelpar and metabolites were determined using validated a LC-MS/MS method.

Following a single oral dose of 10 mg seladelpar, seladelpar AUC increased 1.1-fold in subjects with mild (Child-Pugh A), 2.5-fold in moderate (Child-Pugh B), and 2.1-fold to 2.5-fold in severe (Child-Pugh C) hepatic impairment. Seladelpar Cmax also increased 1.3-fold in subjects with mild (Child-Pugh A), 5.2-fold in moderate (Child-Pugh B), and 5-fold in severe (Child-Pugh C) hepatic impairment.

From results of multiple clinical studies, compared to PBC patients with mild hepatic impairment (Child-Pugh A) without portal hypertension, seladelpar exposures (Cₘₐₓ, AUC) were 1.7 to 1.8-fold higher in PBC patients with mild hepatic impairment with portal hypertension and 1.6 to 1.9-fold higher in PBC patients with moderate hepatic impairment (Child-Pugh B) after a single oral dose of 10 mg seladelpar.

Accumulation ratios were less than 1.2-fold in PBC patients with mild hepatic impairment with portal hypertension and PBC patients with moderate hepatic impairment following 10 mg seladelpar once daily dosing for 28 days.

### Example 3: Drug-Drug Interaction Studies

In vitro, seladelpar is a substrate of CYP2C9, CYP2C8, CYP3A4, and the transporters BCRP, P-gp, OATP1B1, OATP1B3, and OAT3. On the other hand, transporter systems: Seladelpar is not a substrate of MATE1, MATE2-K, OAT1, OCT1, or OCT2.

An open-label, single-dose, 6-arm, fixed-sequence DDI study was conducted to assess the effect of fluconazole (CYP2C9 inhibitor), carbamazepine (CYP3A4 inducer), probenecid (OAT3 inhibitor), quinidine (P-gp inhibitor), cyclosporine (BCRP, OATP1B1, OATP1B3 inhibitor), and on the PK of seladelpar in healthy subjects under fasting conditions.

### Carbamazepine

Seladelpar AUC_{0-inf} was decreased by approximately 44% and Cₘₐₓ by 24% following administration of a single 10 mg seladelpar dose after carbamazepine 300 mg twice daily carbamazepine doses for 7 days in healthy subjects. Carbamazepine (CYP3A and CYP2C9 inducer) dose was escalated from 100 mg via 200 mg over 7 days to 300 mg. Specifically, Carbamazepine (CYP3A and CYP2C9 inducer) dose was escalated from 100 mg twice daily for 3 days followed by 200 mg twice daily for 4 days to 300 mg twice daily.

### Fluconazole

Seladelpar AUC_{0-inf} was increased by 2.4-fold and Cₘₐₓ by 1.4-fold following concomitant use of a single 10 mg seladelpar dose with 400 mg fluconazole (moderate CYP2C9 and CYP3A4 inhibitor) in healthy subjects.

### Cyclosporine

Seladelpar AUC_{0-inf} was increased by 2.1-fold and Cₘₐₓ by 2.9-fold following concomitant use of a single 10 mg seladelpar dose with 600 mg cyclosporine (OATP1B, BCRP, and CYP3A inhibitor) in healthy subjects.

### Probenecid

Seladelpar AUC_{0-inf} was increased by 2-fold and Cₘₐₓ by 4.69-fold following concomitant use of a single 10 mg seladelpar dose with 500 mg probenecid (OAT3 and OATP1B inhibitor) in healthy subjects.

### Strong CYP2C9 Inhibitor

Seladelpar AUC_{0-inf} is predicted to increase by 3.2-fold when co-administered with sulphaphenazole (strong CYP2C9 inhibitor).

### Quinidine

Seladelpar exposures were not significantly altered when a single dose of 600 mg quinidine (P-gp inhibitor) was co-administered in healthy subjects.

### Other Drugs

No clinically significant differences in seladelpar pharmacokinetics were predicted when used concomitantly with strong CYP3A4 inhibitors, or CYP2C8 inhibitors.

In clinical studies, no clinically significant differences in the pharmacokinetics of the following drugs were observed when used concomitantly with seladelpar: tolbutamide (CYP2C9 substrate), midazolam (CYP3A4 substrate), simvastatin (CYP3A4 and OATP substrate), atorvastatin (CYP3A4 and OATP substrate), or rosuvastatin (BCRP and OATP substrate).

Based on in vitro studies, 10 mg seladelpar does not significantly affect the pharmacokinetics of concomitant drugs that are substrates of CYP enzymes (1A2, 2B6, 2C8, 2C19, 2D6, UGTs, P-gp, MATEs, OCT1, OCT2, OAT1, or OAT3).

### Example 3: Pharmacogenomics

CYP2C9 activity is decreased in individuals with genetic variants such as CYP2C9*2 and CYP2C9*3. Compared to CYP2C9 normal metabolizers (*1/*1, n=84) after a single dose of seladelpar 1 mg to 15 mg, dose-normalized AUC_{0-inf} was 48% higher in CYP2C9 poor metabolizers (*2/*3, n=2) and 24% higher in CYP2C9 intermediate metabolizers (*1/*2, *1/*8, *1/*3, *2/*2, n=28). Dose-normalized Cₘₐₓ was similar for CYP2C9 normal, intermediate, and poor metabolizers. Seladelpar pharmacokinetics was not evaluated in patients who are CYP2C9 poor metabolizers with two no function alleles (e.g., *3/*3). CYP2C9 poor metabolizers may have increased AUC when seladelpar is used concomitantly with a moderate to strong CYP3A4 inhibitor.

From clinical studies, the prevalence of CYP2C9 poor metabolizers is approximately 2 to 3% in White populations, 0.5 to 4% in Asian populations, and <1% in African American populations. Additional decreased or nonfunctional alleles (e.g., *5, *6, *11) are more prevalent in African American populations.

### Example 4: Nonclinical Toxicology

### Carcinogenesis

In a 2-year study in CD-1 mice, oral administration of seladelpar produced hepatocellular adenoma or carcinoma at a dose of 5 mg/kg/day in males (6-times the recommended dose based on AUC) and 20 mg/kg/day in females (140-times the recommended dose based on AUC). No tumorigenic effects were observed in female mice at doses of up to 10 mg/kg/day (49-times the recommended dose based on AUC).

In a 2-year study in Sprague-Dawley rats, oral administration of seladelpar produced benign interstitial cell tumors in testes and squamous cell carcinoma of the nonglandular stomach in males at a dose of 30 mg/kg/day (79-times the recommended dose based on AUC). No tumorigenic effects were observed in males at doses of up to 10 mg/kg/day (14-times the recommended dose based on AUC) or in females at doses of up to 30 mg/kg/day (26-times the recommended dose based on AUC).

### Mutagenesis

Seladelpar was negative in the in vitro bacterial reverse mutation (Ames) assay, the in vitro mouse lymphoma assay, and the in vivo mouse micronucleus test.

### Impairment of Fertility

Seladelpar had no effects on fertility or reproductive function in male and female rats at oral doses of up to 100 mg/kg/day (271-times and 115-times the maximum recommended dose in male and female rats, respectively, based on AUC).

Animal Toxicology and/or Pharmacology

In a 2-year study in CD-1 mice, seladelpar produced an increased incidence of lens cataracts at 5 mg/kg/day in both sexes (6-times and 19-times the recommended dose in male and female mice, respectively, based on AUC). In a 2-year study in Sprague-Dawley rats, seladelpar produced an increased incidence of cornea inflammation at 10 mg/kg/day (14-times the recommended dose based on AUC) and cornea mineralization at 30 mg/kg/day (79-times the recommended dose based on AUC), with both effects observed in males only. The incidence of cornea inflammation or cataract was not increased in male rats at 3 mg/kg/day (5-times the recommended dose based on AUC).

### Example 5: Adverse Reactions

In a Phase 3 clinical trial (“Trial 1”), 193 patients were randomized to receive either seladelpar 10 mg (n=128) or placebo (n=65) once daily for 12 months. Seladelpar or placebo was administered in combination with UDCA in 94% of patients and as monotherapy in 6% of patients who were unable to tolerate UDCA.

### Common Adverse Reactions

TABLE 1

| | Seladelpar 10 mg | | |
|---|---|---|---|
| | Once daily | | Placebo |
| | (N = 128) | | (N = 65) |
| Adverse Reaction ^{a} | % (n) | | % (n) |
| Headache | 8% | (10) | 3% (2) |
| Abdominal Pain ^{b} | 7% | (9) | 2% (1) |
| Nausea ^{b} | 6% | (8) | 5% (3) |
| Abdominal Distension ^{b} | 6% | (8) | 3% (2) |
| Dizziness | 5% | (6) | 2% (1) |

^{a} Occurring in greater than or equal to 5% of patients in the seladelpar treatment arm and at an incidence greater than or equal to 1% higher than in the placebo treatment arm.
^{b} The gastrointestinal adverse reactions were mild to moderate without the need for discontinuation of seladelpar.

### Fractures

Fractures occurred in 54% (n=65) of seladelpar-treated patients compared to no placebo-treated patients. Baseline bone mineral density was not obtained. The median time to fracture after receiving seladelpar was 295 days (interquartile range: 89-349).

### Less Common Adverse Reactions

Additional adverse reactions that occurred more frequently in the seladelpar-treated patients compared to placebo, but in less than 5% of patients, included alopecia, rash, dyspepsia, rash, alopecia, vomiting, anemia, and cough.

### Laboratory Abnormalities-Estimated Glomerular Filtration Rate

Seladelpar-treated patients developed decreased estimated glomerular filtration rate (eGFR) (serum creatinine elevations) more frequently compared to placebo-treated patients. Ten percent (n=12) of seladelpar-treated patients had a decline in eGFR of at least 25%, compared to 2% (n=1) of placebo-treated patients. None of the patients experienced an eGFR decline of 50% or more. The decline in eGFR stabilized or returned towards baseline with ongoing seladelpar treatment. None of the patients required discontinuation of seladelpar and there were no clinical findings associated with the observed changes in eGFR.

### Example 6: Efficacy of Seladelpar

In Trial 1, the efficacy of seladelpar was evaluated in a 12-month, randomized, double-blind, placebo-controlled trial. The study included 193 adult patients with PBC with an inadequate response or intolerance to UDCA. Patients were included in the trial if their ALP was greater than or equal to 1.67-times the upper limit of normal (ULN) and total bilirubin (TB) was less than or equal to 2-times the ULN. Patients were excluded from the trial if they had other chronic liver diseases, clinically important hepatic decompensation including portal hypertension with complications, or cirrhosis with complications (e.g., Model for End Stage Liver Disease [MELD]score of 12 or greater, known esophageal varices or history of variceal bleeds, history of hepatorenal syndrome).

Patients were randomized to receive seladelpar 10 mg (n=128) or placebo (n=65) once daily for 12 months. Seladelpar or placebo was administered in combination with UDCA in 181 (94%) patients during the trial, or as a monotherapy in 12 (6%) patients who were unable to tolerate UDCA.

### Baseline Demographics and Characteristics

The mean age of patients was 57 (Range: 28 to 75) years; 95% were female; 88% were White, 6% Asian, 2% Black or African American, and 3% American Indian or Alaska Native. Twenty-nine percent of the patients, 23% in the seladelpar 10 mg arm and 42% in the placebo arm identified as Hispanic/Latino. Thirty-three percent of the patients, 39% in the seladelpar 10 mg arm and 20% in the placebo arm, were enrolled in the US.

At baseline, 18 (14%) of the seladelpar-treated patients and 9 (14%) of the placebo-treated patients met at least one of the following criteria: Fibroscan >16.9 kPa, historical biopsy or radiological evidence suggestive of cirrhosis, platelet count <140,000/μL with at least one additional laboratory finding including serum albumin <3.5 g/dL, INR>1.3, or TB>1-time ULN; or clinical determination of cirrhosis by the investigator.

The mean baseline ALP concentration was 314 (Range: 161 to 786) units per liter (U/L), corresponding to 2.7-times ULN. The mean baseline TB concentration was 0.8 (Range: 0.3 to 2.0) mg/dL and was less than or equal to the ULN in 87% of the patients. Other mean baseline liver biochemistries were 48 (Range: 9 to 115) U/L for ALT and 40 (Range: 16 to 94) U/L for AST.

### Biochemical Results

The primary endpoint was biochemical response at Month 12, where biochemical response was defined as achieving ALP less than 1.67-times ULN, an ALP decrease of greater than or equal to 15% from baseline, and TB less than or equal to ULN. ALP normalization (i.e., ALP less than or equal to ULN) at Month 12 was a key secondary endpoint. The ULN for ALP was defined as 116 U/L. The ULN for TB was defined as 1.1 mg/dL.

Table 2 presents results at Month 12 for the percentage of patients who achieved biochemical response, achieved each component of biochemical response, and achieved ALP normalization. Seladelpar demonstrated greater improvement on biochemical response and ALP normalization at Month 12 compared to placebo. Overall, 87% of patients had a baseline of TB concentration less than or equal to ULN. Therefore, improvement in ALP was the main contributor to the biochemical response rate results at Month 12. Patients who discontinued treatment prior to Month 12 or who had missing data were considered as non-responders.

TABLE 2

| | Seladelpar 10 mg | | | | Treatment | |
|---|---|---|---|---|---|---|
| | once daily | | Placebo | | Difference % | |
| | (N = 128) | | (N = 65) | | (95% CI) ^{b} | |
| Biochemical Response | 79 | (62) | 13 | (20) | 42 | (28, 53) |
| Rate, n (%)^{a, c} | | | | | | |
| Components of | | | | | | |
| Biochemical Response | | | | | | |
| ALP less than 1.67-times | 84 | (66) | 17 | (26) | 39 | (25, 52) |
| ULN, n (%) | | | | | | |
| Decrease in ALP of at least | 107 | (84) | 21 | (32) | 51 | (37, 63) |
| 15%, n (%) | | | | | | |
| TB less than or equal to | 104 | (81) | 50 | (77) | 4 | (−7, 17) |
| ULN, n (%) | | | | | | |
| ALP Normalization, n | 32 | (25) | 0 | (0) | 25 | (18, 33) |
| (%)^{c, d} | | | | | | |

^{a}Biochemical response is defined as ALP less than 1.67-times ULN, an ALP decrease of greater than or equal to 15%, and TB less than or equal to ULN.
^{b} 95% unstratified Miettinen and Nurminen confidence intervals (CIs) are provided.
^{c}p < 0.0001 for seladelpar 10 mg versus placebo. P-values were obtained using the Cochran-Mantel-Haenszel test stratified by baseline ALP level (<350 U/L versus ≥350 U/L) and baseline pruritus NRS (<4 versus ≥4).
^{d}ALP normalization is defined as ALP less than or equal to ULN.

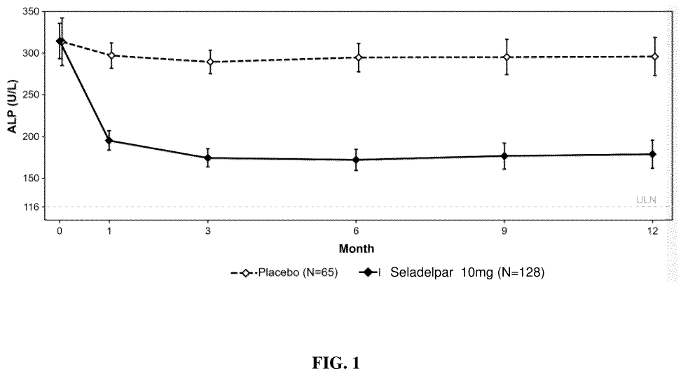

FIG. 1 shows the mean (95% CI) levels of alkaline phosphatase (ALP) over 12 months. There was a trend of lower ALP in seladelpar arm compared to placebo arm starting at Month 1 through Month 12. Specifically, FIG. 1 presents means and 95% Wald CIs for baseline, and least squares means and corresponding 95% CIs based on a mixed-effect model for repeated measures (MMRM) for months 1, 3, 6, 9 and 12. The MMRM adjusts for baseline ALP, baseline ALP level (<350 U/L versus >350 U/L), baseline pruritus NRS (<4 versus ≥4), time (in months), treatment arm, treatment-by-baseline ALP interaction, and treatment-by-time interaction. The least squares mean change from baseline in ALP at month 12 was −137 (−153, −120) U/L and −20 (−43, 4) U/L in the seladelpar 10 mg and placebo arms, respectively, wherein ULN represents upper limit of normal.

Biochemical response at month 3 comparing seladelpar as a monotherapy to placebo was evaluated in a pooled analysis of a subset of patients from Trial 1 and another randomized, double-blind, placebo-controlled trial in a similar patient population. There was a trend of improvement on biochemical response at month 3 in the seladelpar monotherapy group compared to the placebo group.

### Pruritus

A single-item patient-reported outcome (PRO), the pruritus Numerical Rating Scale (NRS), evaluated patients' daily worst itching intensity on an 11-point rating scale with scores ranging from 0 (“no itching”) to 10 (“worst itching imaginable”) in Trial 1. The pruritus NRS was administered daily in a 14-day run-in period prior to randomization through Month 6.

Seladelpar and placebo were compared, on the key secondary endpoint evaluating the change from baseline in monthly pruritus score through Month 6 in patients with baseline average pruritus scores greater than or equal to 4. The baseline average pruritus score for each patient was calculated by averaging the pruritus NRS scores administered in the 14-day run-in period. The monthly pruritus scores for each patient for post-baseline months were calculated by averaging the pruritus NRS scores within the last week in the month. Patients treated with seladelpar demonstrated greater improvement in pruritus compared with placebo. This effect was sustained through Month 12 of treatment.

Table 3 presents the results of the comparison between seladelpar and placebo were compared, on the key secondary endpoint evaluating the change from baseline in pruritus score through Month 6 in patients with baseline average pruritus scores greater than or equal to 4. The baseline average pruritus score for each patient was calculated by averaging the pruritus NRS scores administered in the 14-day run-in period and on Day 1 before treatment initiation. The pruritus scores at Month 6 for each patient were calculated by averaging the pruritus NRS scores within the last week in the month. Patients treated with seladelpar demonstrated greater improvement in pruritus compared with placebo.

TABLE 3

| | |
|---|---|
| Seladelpar 10 mg | |
| Once Daily | Placebo |
| (N = 49) | (N = 23) |

| | | | | |
|---|---|---|---|---|
| Baseline Average Pruritus | 6.1 | (1.4) | 6.1 | (1.4) |
| Score, Mean (SD) | | | | |

Change from Baseline in Pruritus Score at Month 6^{a}

| | | | | |
|---|---|---|---|---|
| Mean (SE) | −3.2 | (0.3) | −3.2 | (0.3) |

| | |
|---|---|
| Mean difference vs. Placebo | −1.5 (−2.5, −0.5) |
| (95% CI) | p = 0.0051 |

^{a}Based on least square means from a mixed-effect model for repeated measures (MMRM) for change from baseline at Months 1 (Week 4), 3 (Week 12), and 6 (Week 26) accounting for baseline average pruritus score, baseline ALP level (<350 U/L versus ALP level ≥350 U/L), treatment arm, time (in months), and treatment-by-time interaction.

Results were analyzed based on least square (LS) means from a mixed-effect model for repeated measures (MMRM) for change from baseline at Months 1 (Week 4), 3 (Week 12), and 6 (Week 26) accounting for baseline average pruritus score, baseline ALP level (<350 U/L versus ALP level≥350 U/L), treatment arm, time (in months), and treatment-by-time interaction. At baseline, the mean (SD) pruritus NRS score was 6.1 (1.4) and 6.6 (1.4) in the seladelpar 10 mg (N=49) and placebo (N=23) arms, respectively. The mean (SE) change from baseline at Month 6 was −3.2 (0.3) and −1.7 (0.4) in the seladelpar 10 mg and placebo arms, respectively (mean difference [95% CI]: −1.5 [−2.5, −0.5]).

### Example 7: Changes of Metabolism Markers Levels after Seladelpar Administration

In this Example, untargeted global serum metabolomics were used to gain mechanistic insight into metabolic pathways that were altered by seladelpar treatment to explain the improvement in prognostic markers seen in patients with PBC in clinical studies, including those noted in preceding Examples.

In a phase 3, randomized, double-blind, placebo-controlled clinical study of seladelpar in patients with PBC and an inadequate response to or an intolerance to ursodeoxycholic acid (UDCA) (“Trial 2”), patients aged 18-75 years, diagnosed with PBC, and either showing an incomplete response or intolerance of UDCA were recruited. Eligible patients had ALP level greater than 1.67 times the upper limit of the normal range (ULN) despite stable doses of UDCA for the past 1 year, if tolerated, and total bilirubin level of less than 2 times the ULN. Patients were randomly assigned in a 1:1:1 ratio, to receive once-daily oral placebo, seladelpar 5 mg or 10 mg. The serum metabolome of patients was examined using fasting serum samples collected on day 1 and after 12 weeks of treatment with placebo, seladelpar 5 mg or 10 mg.

For untargeted metabolomic analysis samples were extracted with methanol to precipitate protein and dissociate small molecules bound to protein or trapped in the precipitated protein matrix. The resulting extract containing chemically diverse metabolism markers was dried then reconstituted in solvents. Metabolism markers were identified by comparison to a referenced library of chemical standards. Metabolism markers levels were quantified by area-under-the-curve analysis for peak area. To ensure high quality of the dataset, control and curation processes were subsequently used to ensure true chemical assignment and remove artefacts and background noise. In this Example, “metabolite” may refer to metabolism markers.

Serum samples were also analyzed for carnitine and 17 acylcarnitines by LC-MS/MS: carnitine, acetyl carnitine (C2:0), propionyl carnitine (C3:0), butyryl carnitine (C4:0), isobutyryl carnitine (C5:0), valeryl carnitine (C6:0), isovaleryl carnitine (C5:0), 2-methylbutyryl carnitine (C4:0), 3-hydroxybutyryl carnitine (C4:0), hexanoyl carnitine (C6:0), octanoyl carnitine (C8:0), decanoyl carnitine (C10:0), dodecanoyl carnitine (C12:0), myristoyl carnitine (C14:0), palmitoyl carnitine (C16:0), linoleoyl carnitine (C18:2), oleoyl carnitine (C18:1), and stearoyl carnitine (C18:0). Quantitation was performed using a weighted linear least squares regression analysis generated from fortified calibration standards prepared concurrently with study samples.

To explore whether seladelpar affects the expression levels of carnitine transporter, carnitine synthesis and mitochondrial shuttle genes, OCTN2, CPT1A, CPT2, CACT, CRAT and BBOX1 from primary human hepatocytes incubated with 10 μM seladelpar levels were measured for 24 hours. Male C57BL/6J mice were orally dosed with vehicle or seladelpar (10 mg/kg/day) for 12 weeks and RNA-seq was performed using terminal tissues (intestine, liver, kidney and skeletal muscle). The same genes were also examined for expression level of the mouse orthologs. PrimeTime™ qPCR primer assays (Integrated DNA Technologies) for RT-qPCR analysis were used. Gene expression levels were calculated using the ΔΔCt method.

A total of 160 patients with PBC who had evaluable serum samples from both Day 1 and Week 12, were included in the serum metabolomics analysis. The patients had been randomly assigned to one of three treatment groups: placebo (n=55), seladelpar 5 mg (n=52) or seladelpar 10 mg (n=53). Demographics and pre- and post-treatment biochemistry are summarized in Table 4. Demographics and serum biochemistry were well balanced among treatment groups. The majority of PBC patients were female (94%), with a mean age of 56 years and BMI of 29 kg/m². They had elevated alkaline phosphatase (ALP) levels (mean 274 U/L), 90% were antimitochondrial antibody-positive, and 95% were on UDCA.

TABLE 4

| | Placebo | Seladelpar 5 mg | Seladelpar 10 mg |
|---|---|---|---|
| Demographics | (n = 55) | (n = 52) | (n = 53) |
| Female, n (%) | 54 (98%) | 47 (90%) | 50 (94%) |
| Age, years | 56 (7) | 56 (9) | 57 (10) |
| BMI, kg/m2 | 29 (6) | 28 (5) | 28 (7) |
| White, n (%) | 49 (89%) | 49 (94%) | 46 (87%) |
| AMP-positive, n (%) | 49 (89%) | 48 (92%) | 47 (89%) |
| UDCA received, n (%) | 54 (98%) | 49 (94%) | 49 (92%) |

| Biochemistry | Day 1 | Week 12 | Day 1 | Week 12 | Day 1 | Week 12 |
|---|---|---|---|---|---|---|
| ALP (U/L) | 282 (105) | 281 (116) | 282 (126) | 179 (56) | 263 (96) | 147 (56) |
| ALT (U/L) | 41 (20) | 43 (23) | 47 (24) | 35 (18) | 42 (20) | 38 (26) |
| AST (U/L) | 35 (14) | 36 (16) | 39 (18) | 35 (16) | 38 (14) | 38 (19) |
| GGT (U/L) | 200 (153) | 203 (177) | 204 (163) | 141 (112) | 208 (154) | 134 (111) |
| Total bilirubin (mg/dL) | 0.7 (0.3) | 0.7 (0.3) | 0.7 (0.3) | 0.7 (0.3) | 0.6 (0.3) | 0.6 (0.3) |
| Total cholesterol | 233 (53) | 231 (53) | 215 (45) | 209 (45) | 231 (56) | 221 (55) |
| (mg/dL) | | | | | | |
| LDL-cholesterol | 137 (42) | 136 (42) | 120 (36) | 113 (33) | 129 (43) | 120 (45) |
| (mg/dL) | | | | | | |
| HDL-cholesterol | 73 (22) | 72 (23) | 75 (22) | 77 (25) | 75 (22) | 80 (22) |
| (mg/dL) | | | | | | |
| Triglycerides (mg/dL) | 117 (55) | 116 (44) | 101 (48) | 96 (43) | 125 (77) | 101 (55) |
| Albumin (g/dL) | 4.2 (0.2) | 4.2 (0.3) | 4.1 (0.3) | 4.2 (0.3) | 4.1 (0.3) | 4.2 (0.3) |
| Total protein (g/dL) | 7.6 (0.6) | 7.5 (0.6) | 7.4 (0.4) | 7.4 (0.4) | 7.5 (0.6) | 7.6 (0.6) |
| Urea (mg/dL) | 15.4 (3.9) | 14.7 (3.8) | 15.5 (4.6) | 15.8 (4.0) | 14.9 (4.9) | 15.7 (4.5) |
| Creatinine (mg/dL) | 0.71 (0.13) | 0.72 (0.13) | 0.72 (0.18) | 0.75 (0.16) | 0.76 (0.23) | 0.79 (0.16) |

A total of 1474 metabolites (1171 named and 303 unnamed) were identified in the global untargeted analysis of human serum from patients with PBC. Metabolite abundances were compared between Day 1 and Week 12 samples within each treatment group—placebo, seladelpar 5 mg, and seladelpar 10 mg—using Patient ID as a random effect to account for paired samples. After 12 weeks of treatment, 5 metabolites showed significant changes in the placebo group, 108 metabolites in the seladelpar 5 mg group, and 456 metabolites in the seladelpar 10 mg group (FDR<0.05) (Table 5).

TABLE 5

| | | Seladelpar | Seladelpar |
|---|---|---|---|
| | Placebo | 5 mg | 10 mg |
| Total biochemicals, p < 0.05 | 5 | 108 | 456 |
| biochemicals | 2 \| 3 | 56 \| 52 | 194 \| 262 |
| (increased\|decreased) | | | |

Of the metabolites that were significantly changed in the seladelpar 5 mg group, 92 were also significant in the seladelpar 10 mg group. Of these, 75 exhibited a greater fold change in the seladelpar 10 mg group, indicating dose-dependency in metabolite levels. The most common and significantly increased metabolites following seladelpar treatment were carnitine, acylcarnitines and branched-chain amino acid catabolites. In contrast, seladelpar treatment significantly decreased the levels of dicarboxylates, fatty acids and ceramides.

Random Forest analysis was used to identify metabolites that differentiated Day 1 and Week 12 samples following treatment with seladelpar. The Random Forest model showed predictive accuracy of 81% for the 5 mg and 93.4% for the 10 mg seladelpar groups. Among the top 30 metabolites ranked by their importance for the effect of seladelpar 10 mg treatment, the most common included dicarboxylates, carnitines (both free carnitine and acylcarnitines) and branch-chain amino acid metabolites. The metabolite that most differentiated the seladelpar 10 mg treated serum samples was N,N,N-trimethyl-5-aminovalerate (TMAVA or 5-aminovaleric acid betaine), followed by 2,3-dihydroxy-2-methylbutyrate, carnitine and dodecanedioate (C12-DC). Random Forest classification also identified 40% of metabolites in common between the 5 mg and 10 mg seladelpar treatments.

Weighted correlation network analysis (WCNA) was used to identify metabolite modules that are co-abundant between baseline and treated samples. By analyzing correlations among metabolites across samples, WCNA revealed metabolic patterns that were not apparent through single-metabolite or differential abundance analyses. Ten distinct metabolite modules were identified, each representing groups of highly interconnected metabolites. Module 1, which consists of 416 metabolites, showed clear differences between baseline, placebo and seladelpar treated groups, as indicated by the module's eigen-metabolite values. Module 1 metabolites showed treatment-specific changes in metabolite abundances, and many of which displayed a dose-dependent response. The use provided deeper insights into how seladelpar treatment impacts diverse metabolic pathways, including those involving dicarboxylates, acylcarnitines, ceramides, branch-chain amino acid catabolites, lipids, gamma glutamyl amino acids and unnamed metabolites.

Differential abundance analysis, Random Forest and WCNA identified an overlapping set of key metabolites affected by seladelpar treatment, leading a broad range of changes in the abundance of several metabolite classes, including dicarboxylates, carnitines, branched-chain amino acid catabolites, fatty acids, ceramides and others.

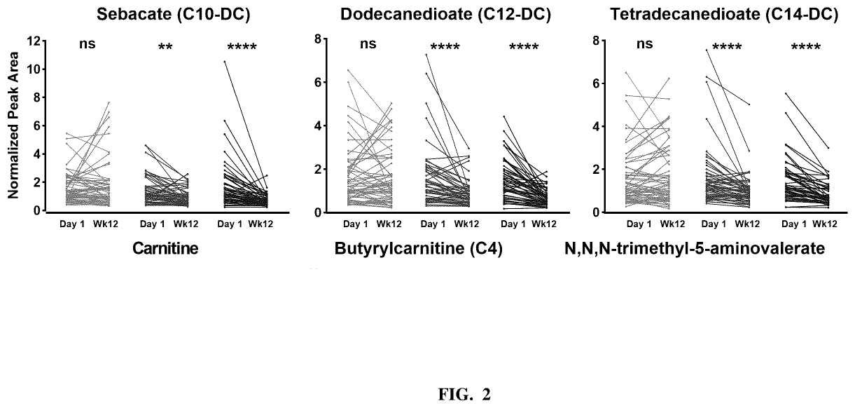

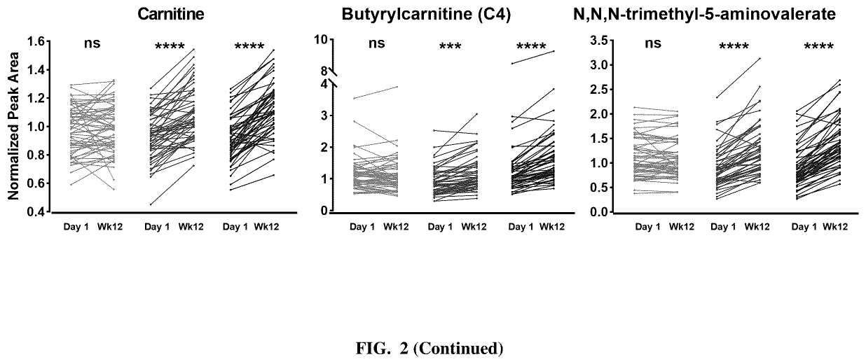

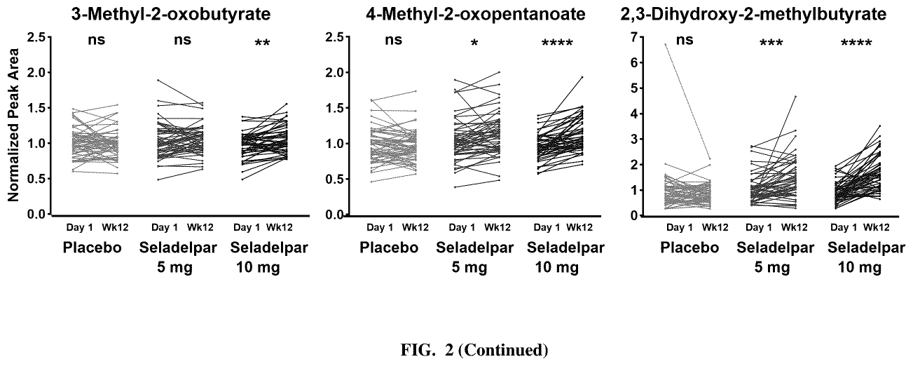

Random Forest, WCNA and differential abundance analyses of the data revealed overlapping patterns of metabolite changes following seladelpar treatment in PBC patients. In this clinical trial, which tested 5 mg and 10 mg doses of seladelpar, we found that many of the prominent metabolite changes were dose dependent. At the 10 mg dose (the currently prescribed dose), 17% of the identified metabolites significantly increased, while 22% decreased with seladelpar treatment. Representative examples of changes in dicarboxylates, carnitines, carnitine-related metabolites and branched-chain amino acid catabolites in individual PBC patients are shown in FIG. 2. As shown in FIG. 2, seladelpar treatments resulted in substantial changes in dicarboxylates (e.g., sebacate, dodecanedioate, tetradecanediote), carnitine, butyryl carnitine (C4), N,N,N-trimethyl-5-aminovalerate and branched-chain amino acid catabolites (3-Methyl-2-oxobutyrate, 4-methyl-2-oxopentanoate, 2,3-dihydrox-2-methylbutyrate) levels from Day 1 to Week 12 in individual PBC patients. Statistical significance was determined by the Benjamini-Hochberg method (ns: FDR≥0.05, *FDR<0.05, **FDR<0.01, ***FDR<0.001 and ****FDR<0.0001).

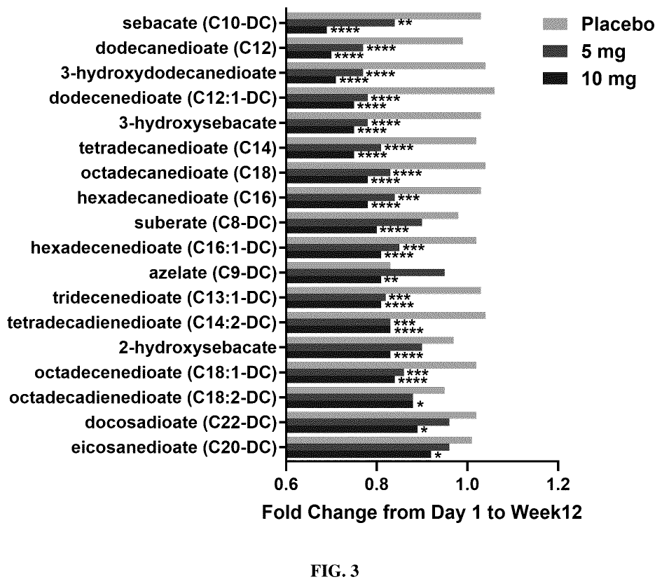

One of the most notable changes following seladelpar treatment detected by differential abundance, Random Forest, and WCNA were in the decrease in dicarboxylates. In Random Forest analysis, six of the top 30 metabolites in the seladelpar 10 mg group were dicarboxylate species, including dodecanedioate (C12-DC), sebacate (C10-DC), octadecanedioate (C18-DC), tetradecanedioate (C14-DC), hexadecanedioate (C16-DC) and dodecenedioate (C12:1-DC). WCNA also identified 12 out of 29 hub metabolites as dicarboxylates. Dicarboxylates are derived from diet or o-oxidation of free fatty acids. FIG. 3 shows fold changes of dicarboxylate species with varying chain lengths detected by differential abundance from Day 1 to Week 12. Statistical significance was determined by the Benjamini-Hochberg method (ns: FDR >0.05, *FDR<0.05, **FDR<0.01, ***FDR<0.001 and ****FDR<0.0001). As shown in FIG. 3, among the 18 dicarboxylate species with varying chain lengths detected by differential abundance, seladelpar 10 mg significantly decreased all 18, and 15 dicarboxylates were significantly decreased by seladelpar 5 mg treatment while none were significantly changed by placebo treatment. Changes in sebacate (C10-DC), dodecanedioate (C12-DC) and tetradecanedioate (C14-DC) in individual PBC patients are shown in FIG. 2.

Carnitine and acylcarnitines play key roles in facilitating fatty acid 3-oxidation in mitochondria and peroxisomes, helping to remove excess and potentially toxic lipid species that result from mitochondrial dysfunction in PBC patients. In the Random Forest and WCNA of seladelpar treatment, carnitines were the second most commonly identified metabolites. Among the 62 carnitine species detected, 30 showed a significant increase after treatment with 10 mg of seladelpar (as shown by differential abundance) with a clear dose-dependent response, unlike the placebo group, where no changes were observed.

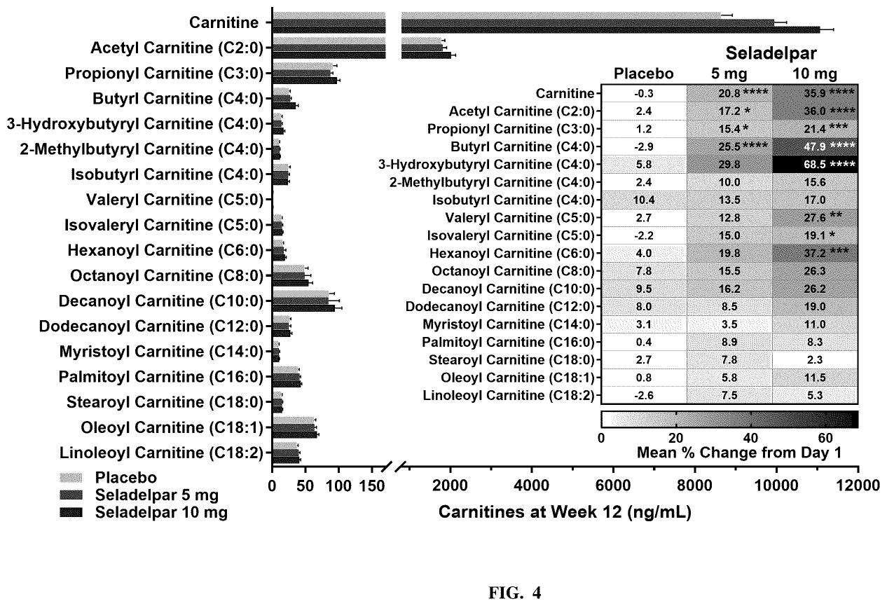

Targeted measurements of carnitine and acylcarnitines confirmed the findings of the untargeted analysis shown in FIG. 4. FIG. 4 shows carnitine and acylcarnitine levels at Week 12. The inset heatmap in FIG. 4 illustrates the percent change in carnitine and acylcarnitine levels from Day 1. FIG. 5 shows changes in carnitine, acetyl carnitine and 3-hydroxybutyryl carnitine levels in individual PBC patients. As shown in FIGS. 4 and 5, seladelpar treatment led to a dose-dependent increase in both carnitine and shorter-chain acylcarnitines, with carnitine and acetylcarnitine being the most abundant species. After 12 weeks of seladelpar treatment, carnitine and acetylcarnitine levels increased significantly by 36% (p<0.0001) compared to Day 1.

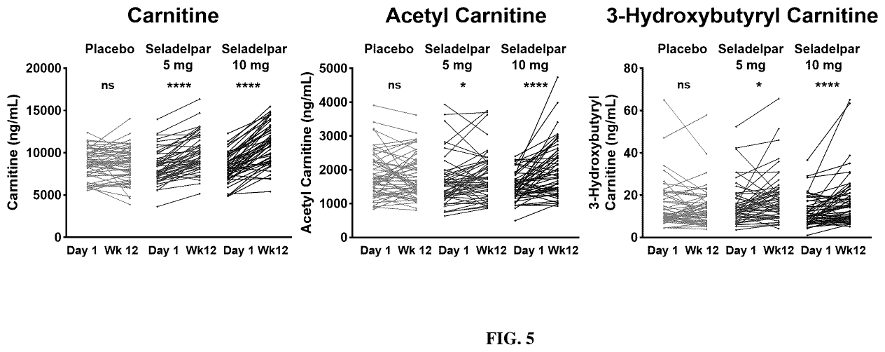

Carnitine homeostasis is primarily maintained through dietary absorption, and by a modest biosynthesis rate from lysine and methionine, and by efficient renal reabsorption. The metabolite N,N,N-trimethyl-5-aminovalerate was identified as the most important differentiating metabolite by Random Forest analysis in the seladelpar 10 mg group, showing a dose-dependent increase. This metabolite is structurally similar to carnitine and serves as a substrate for the plasma membrane carnitine transporter OCTN2 (SLC22A5), which is ubiquitously present in tissues. OCTN2 plays a critical role in the absorption of carnitine from diet and its distribution to tissues for fatty acid 3-oxidation. Based on these findings, OCTN2 was investigated as a potential target gene for seladelpar treatment. Human primary hepatocytes were incubated with 10 μM seladelpar for 24 hours, and qPCR was performed. Data are presented as fold change over vehicle. As shown in FIG. 6, in primary human hepatocytes, seladelpar increased the expression of OCTN2 and genes involved in mitochondrial carnitine shuttle (CPT1A, CPT2, CACT and CRAT), while no change was observed in y-butyrobetaine dioxygenase (BBOX1), the rate-limiting enzyme of carnitine biosynthesis.

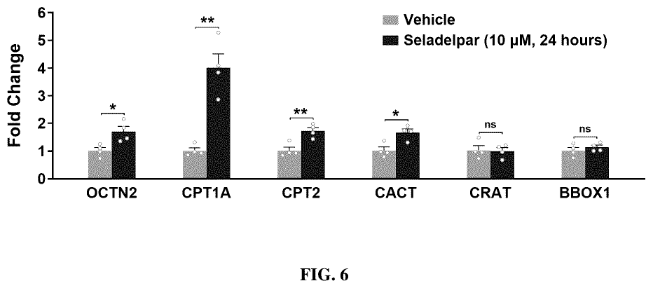

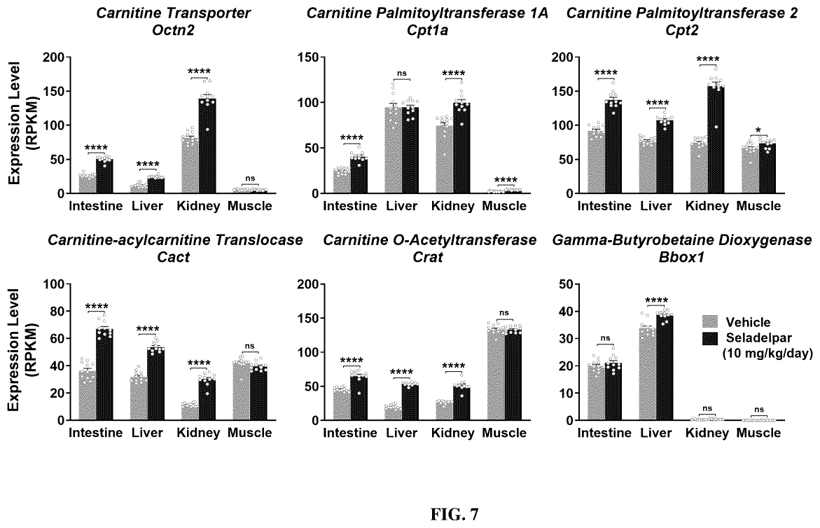

As a mouse model of diet-induced obesity, male C57BL/6J mice were orally dosed with vehicle or seladelpar (10 mg/kg/day) for 12 weeks and RNA-seq was performed using terminal tissues (intestine, liver, kidney and skeletal muscle). In FIG. 7, RNA-seq data are presented as Reads Per Kilobase per Million mapped reads (RPKM). Data are presented as mean±SE. Statistical significance was determined by a paired or unpaired Student's t-test or one-way ANOVA, where appropriate. Statistical significance is indicated as ns, not significant; *, P<0.05; **, P<0.01; ***, P<0.001; and ****, P<0.0001. As shown in FIG. 7, in the mouse model of diet-induced obesity, seladelpar upregulated expression of Octn2 and mitochondrial shuttle genes in the intestine, liver, and kidney. It is contemplated that the observed increase in carnitine levels following seladelpar treatment is due to enhanced dietary carnitine uptake and its reabsorption through OCTN2 upregulation. Thus, seladelpar's upregulation of the carnitine transporter expression in the liver, intestine, and kidney may contribute to the increase in serum carnitine levels. Overall, the broad upregulation of OCTN2 by seladelpar may help boost intracellular carnitine levels to support enhanced fatty acid 3-oxidation.

Overall, the most prominent class of increased metabolites was carnitines, which we confirmed to be elevated by targeted quantitation. Free carnitine and acetylcarnitine levels were also elevated with 10 mg seladelpar treatment, as was N,N,N-trimethyl-5-aminovalerate (TMAVA), which is also a substrate of the carnitine transporter OCTN2. The consistent and significant increase in carnitine and TMAVA levels may be due to seladelpar's upregulation of OCTN2. TMAVA was the most differentiating molecule affected by seladelpar treatment in the Random Forest and could serve as a promising pharmacodynamic marker for seladelpar. The upregulation of OCTN2 and mitochondrial shuttle genes, along with the resulting increase in carnitine levels, likely contribute to the improved capacity for fatty acid 3-oxidation by seladelpar treatment.

Other major classes of metabolites increased by seladelpar treatment included N-acetylamines, N-acetyl amino acids and catabolites of branched-chain amino acids. The branched-chain amino acids—leucine, isoleucine and valine—are essential constituents of proteins and serve as crucial energy sources. Branched-chain amino acids are first catabolized by branched-chain aminotransferase (BCAT) to their α-keto acid intermediates, which are then further catabolized by mitochondrial branch-chain α-keto acid dehydrogenase (BCKDH) complex. This process leads to either anabolic pathways (such as gluconeogenesis or fatty acid synthesis) or energy generation pathways (e.g., the TCA cycle). While the branched-chain amino acids themselves did not show significant changes, there were significant and dose-dependent increases in their catabolites after seladelpar treatment. The branched-chain amino acid catabolite 2,3-dyhydroxy-2-methylbutyrate was identified as the second most important metabolite by Random Forest analysis and a key hub metabolite by WCNA of seladelpar 10 mg group. Other branched-chain amino acid catabolites, including 3-methyl-2-oxobutyrate, 3-methyl-2-oxovalerate and 4-methyl-2-oxopentanoate, also showed increased differential abundance after 12 weeks of seladelpar 10 mg treatment as shown in FIG. 2.

Treatment with seladelpar 10 mg for 12 weeks resulted in significant decreases in all the saturated fatty acids detected (C1O, C12, C14, C15, C16, C17, C18, C19 and C20) and many unsaturated fatty acid species. Seladelpar 5 mg treatment showed a trend toward decreasing both saturated and unsaturated fatty acids, but these changes did not reach statistical significance. No significant changes in fatty acid levels were observed in the placebo group.

Ceramides have been shown to have pathological consequences when present at elevated levels in serum. As found for the dicarboxylates and fatty acids, 7 out of 10 detected ceramides were significantly reduced by seladelpar 10 mg treatment. These ceramides include N-stearoyl-sphinganine (d18:0/18:0), ceramide (d18:1/17:0, d17:1/18:0), ceramide (d18:1/14:0, d16:1/16:0), N-palmitoyl-sphinganine (d18:0/16:0), N-palmitoyl-heptadecasphingosine (d17:1/16:0), N-stearoyl-sphingosine (d18:1/18:0) and ceramide (d18:2/24:1, d18:1/24:2). In the seladelpar 5 mg group, there was a trend toward a decrease in ceramide levels, though it was not statistically significant.

Seladelpar treatments led to a decrease in gamma-glutamyl amino acids, which was consistent with reduction of gamma-glutamyl transferase activity seen in PBC patients treated with seladelpar. Additionally, seladelpar 10 mg treatment resulted in increased N-acetylamines. Changes were also observed in other lipid groups, such as phospholipids, lysophospholipids, sphingomyelins, and plasmalogens, with some species within each group increasing and others decreasing. Notably, 12-HETE, an inflammatory lipid mediator derived from arachidonic acid (C20:4), was significantly reduced by seladelpar 10 mg treatment. Plant-derived sterols such as beta-sitosterol and campesterol which serve as markers of cholesterol absorption, were significantly decreased by seladelpar 10 mg treatment.

Glycerol 3-phosphate serves as a substrate for triglyceride synthesis and is a key intermediate in glycolysis. It plays a critical role in the glycerol 3-phosphate shuttle, a pathway that transports electrons produced during glycolysis across the inner mitochondrial membrane for oxidative phosphorylation, by oxidizing cytoplasmic NADH to NAD+. In the Random Forest analysis, glycerol 3-phosphate was identified in both the seladelpar 5 mg and 10 mg groups, where it was significantly increased by seladelpar treatment. Glycerol 3-phosphate, an intermediate in the glycolytic pathway that plays a central role in energy production, was increased significantly by seladelpar and could reflect elevated fat mobilization and lipolysis.

Fatty acid β-oxidation occurs in both mitochondria and peroxisomes, with each organelle containing distinct sets of enzymes encoded by different genes and with different specific substrates. The mitochondrial β-oxidation pathway primarily processes C18 and shorter fatty acids (long-, medium- and short-chain fatty acids), while the peroxisomal pathway handles very long-chain fatty acids (C22 or longer), branched-chain fatty acids, bile acid intermediates and long-chain dicarboxylates. The substantial reduction in dicarboxylates in this study is likely due in part to peroxisomal and mitochondrial 3-oxidation induced by seladelpar. Peroxisomes are only capable of shortening the fatty acid chain length, and the end products of peroxisomal β-oxidation (acetyl-CoA, propionyl-CoA and shortened acyl-CoA) must be transported to the mitochondria for complete oxidation via the TCA cycle and oxidative phosphorylation processes.

The widespread decrease in various lipid classes, including dicarboxylates, saturated and unsaturated fatty acids, and ceramides, following seladelpar treatment may thus be explained by enhanced fatty acid β-oxidation in both mitochondria and peroxisomes. Circulating ceramides have been shown to have negative health consequences, so the finding here that seladelpar lowers most of the MS-identified ceramides is intriguing. Specific lipids that have been shown to associate with initiation and resolution of inflammatory events, such as 12-HETE, 14-HDoHE/17-HDoHE were all reduced by seladelpar.

Cholestasis in PBC is known to cause mitochondrial disturbances, leading to elevated levels of fatty acids and other lipids. The metabolomic data demonstrated that many of these changes can be ameliorated with seladelpar treatment. Mitochondrial disturbances in PBC have been suspected to contribute to the fatigue commonly associated with the disease. In this context, it is contemplated that seladelpar may be able to alleviate this prevalent and disabling symptom of PBC.

### Example 8: Pharmacodynamic Assessment of Seladelpar on Cardiac Repolarization

A Phase 1, randomized, partially-blinded study enrolled healthy subjects aged 18-55 years, who received either a blinded, single, oral dose of seladelpar (10 mg or 200 mg), placebo, or unblinded moxifloxacin 400 mg as a positive control. Continuous 12-lead Holter monitoring, safety electrocardiogram, laboratory assessments, and vital sign measurements were performed for 24 hours post-dose. Plasma samples for pharmacokinetic (PK) analysis were collected in intervals for 96 hours post-dose. The primary endpoint assessed the relationship between time-matched, baseline-adjusted, placebo-corrected QT interval corrected for heart rate using Fridericia's formula (QTcF) and seladelpar plasma concentrations. Safety was assessed via adverse events (AEs) and laboratory findings.

Sixty-two subjects were randomized to seladelpar 10 mg (n=13), seladelpar 200 mg (n=17), placebo (n=12), or moxifloxacin 400 mg (n=20). Seladelpar was rapidly absorbed, with dose-dependent increases in PK parameters. Concentration regression analysis demonstrated no significant QTcF prolongation with seladelpar. At the geometric mean maximum concentration (Cₘₐₓ) of seladelpar 10 mg (83 ng/mL), the estimated ΔΔQTcF (90% CI) was 0.0 msec (−2.9, 3.0). At the geometric mean Cₘₐₓ of seladelpar 200 mg (5705 ng/mL), the estimated ΔΔQTcF (90% CI) was −0.6 msec (−3.9, 2.7). Assay sensitivity was confirmed with moxifloxacin 400 mg. Both doses of seladelpar were well tolerated, with headache (29%) being the most common AE with seladelpar 200 mg. All AEs were mild in severity. One subject in the seladelpar 200 mg group had a transient increase in creatine kinase to >5×the upper limit of normal.

Single doses of seladelpar 10 mg and 200 mg did not prolong the QTcF interval in healthy subjects. The upper bounds of the 90% CIs for the estimated mean ΔΔQTcF at both seladelpar doses (10 mg and 200 mg) were well below a threshold of 10 msec, indicating no clinically meaningful effect on cardiac repolarization.

### Example 9: Attenuation, Near Resolution, and Prevention of Pruritus

In the Phase 3, multicenter, double-blind, randomized, placebo-controlled trial in patients with PBC who had received UDCA for at least 12 months or had a history of unacceptable side effects with UDCA, patients were randomly assigned to oral seladelpar 10 mg daily or placebo for up to 12 months along with UDCA if tolerant.

During the trial, data on pruritus were collected via the Numerical Rating Scale (NRS), the 5-D Itch scale, and the PBC-40. NRS data were collected daily from the run-in visit through 6 months and then for 7 consecutive days during each month through the end of the 12-month treatment period. The 5-D Itch data were collected every 2 weeks from run-in through 6 months, then every 4 weeks through 12 months or until the end of treatment. The PBC-40 data were collected at run-in, randomization, 1 month, 3 months, and then every 3 months through 12 months or until the end of treatment.

To further characterize effects of seladelpar on pruritus in the clinical trial, patient populations were analyzed based on their baseline itch status, including patients who had moderate to severe (NRS≥4) or severe (NRS≥7) pruritus based on the pruritus NRS. Patients with clinically significant pruritus based on the PBC-40 (PBC-40 itch≥7) were also assessed. Lastly, patients with no and near no pruritus (NRS=0 or 1) at baseline were analyzed.

For patients with moderate to severe pruritus at baseline (NRS≥4), mean NRS values over time were assessed to characterize the severity of pruritus over time, and mean changes from baseline in the pruritus NRS was also assessed. Improvements in the NRS by 3 and 4 points at months 6 and 12 in patients with NRS≥4 were provided to characterize the percentage of patients achieving clinically meaningful change. Near resolution of itch by NRS (NRS=0 or 1) was also analyzed. Additionally, additional information on the duration of itch and regions of the body impacted by pruritus based on the 5-D Itch duration and distribution domains were analyzed. From PBC-40 data, change in all domains and the sleep disturbance question at months 6 and 12 were analyzed. Among all patient populations, a “meaningful response” in the PBC-40 domain score per item was defined as a 0.5-point change from baseline.

For patients with severe pruritus at baseline (NRS≥7), mean changes from baseline in the NRS among the NRS≥7 population were analyzed to explore the effects of seladelpar in patients with more severe pruritus at baseline. Near resolution of itch by NRS (NRS=0 or 1) was also assessed. 5-D Itch and PBC-40 data for all domains and the sleep item/question were also analyzed.

For patients with clinically significant pruritus at baseline (PBC-40 itch domain ≥7): Changes in PBC-40 for all domains and the sleep disturbance question were assessed in patients with clinically significant itch at baseline, as was the proportion of patients shifting from PBC-40≥7 to <7 with seladelpar vs placebo.

For overall population, to evaluate patterns of change in NRS across the entire study population, a Sankey plot was generated to show shifts from baseline to 12 months in pruritus NRS by category of no or near no itch (NRS 0-1), mild (NRS>1 to <4), moderate (NRS 4 to <7), or severe itch (NRS≥7). Patients with no itch or near no itch (NRS=0-1) were also specifically evaluated for the development of itch, defined as an NRS increase to ≥2 points by 12 months.

Safety was assessed by treatment-emergent adverse events (AEs; events occurring after initiation of seladelpar and within 30 days of last dose) in patients with moderate to severe itching at baseline (NRS≥4) vs those with less severe or no itching (NRS<4).

In the trial, 128 patients received seladelpar and 65 patients received placebo. Of these patients, 49 in the seladelpar group and 23 in the placebo group had NRS≥4 at baseline. The mean age at diagnosis of those with NRS≥4 was 47 years, and the majority of patients were female and White. Almost all patients with NRS≥4 at baseline had a reported history fatigue (63%, seladelpar; 70%, placebo). Mean NRS score at baseline in the NRS≥4 population was 6.1 in the seladelpar group and 6.6 in the placebo group, and mean PBC-40 itch domain score was 8.7 in the seladelpar group and 9.6 in the placebo group. Mean PBC-40 fatigue domain scores were 31.9 and 34.7 in the NRS≥4 seladelpar and placebo groups, respectively. At baseline, 22% of patients in both of these groups were receiving concomitant antipruritic medications, which included cholestyramine, colestipol, rifampicin, gabapentin, and sertraline.

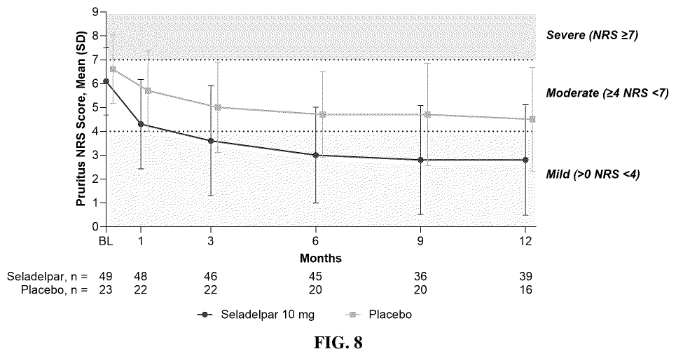

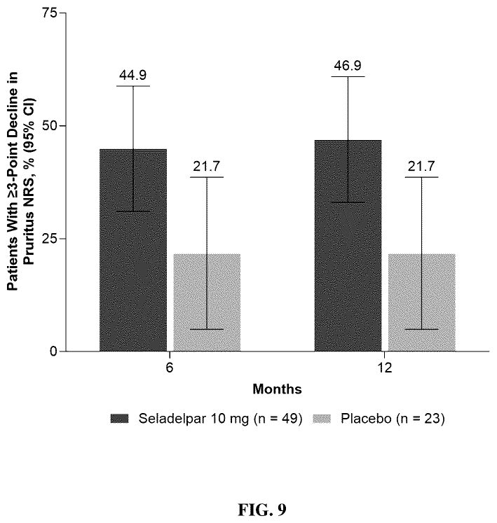

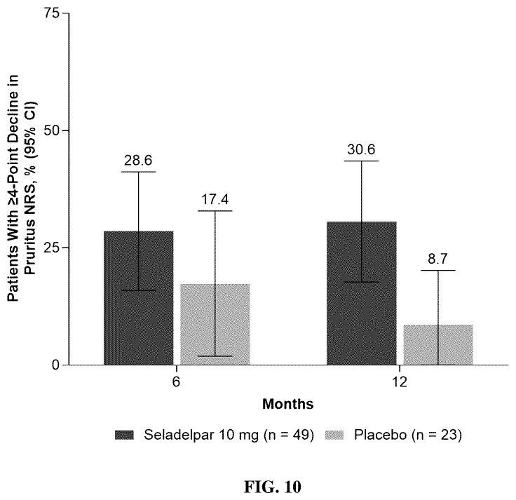

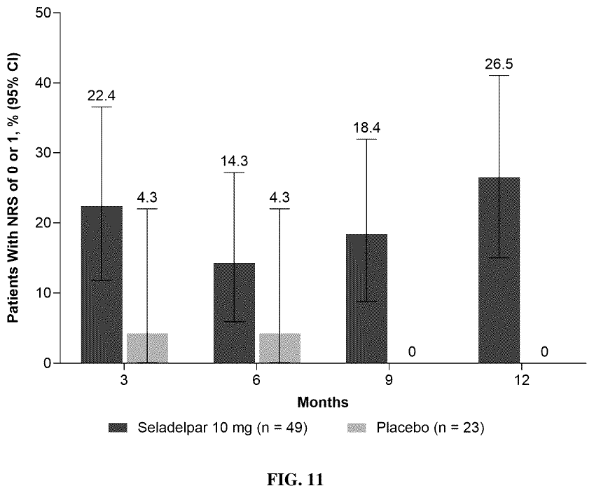

The reduction from baseline in the pruritus NRS score at 6 months was significantly greater in patients receiving seladelpar than in patients receiving placebo (−3.2 vs −1.7; LS mean difference, −1.5; 95% CI, −2.5 to −0.5; P=0.005) among patients with NRS≥4 at baseline. While mean pruritus NRS scores in the placebo group remained in the moderate itch range (NRS≥4 to <7) throughout the duration of the study, mean scores for patients receiving seladelpar fell into the mild itch range (NRS>0 to <4) at 3 months and remained within that range through 12 months of treatment (FIG. 8). For patients with NRS≥4 at baseline, 47% of patients on seladelpar demonstrated a ≥3-point decrease in their pruritus NRS score vs 22% on placebo at 12 months (FIG. 9); a ≥4-point decrease in NRS was achieved by 31% of patients on seladelpar compared with 9% of patients on placebo (FIG. 10). In patients who entered the study with NRS≥4, 27% who received seladelpar demonstrated near resolution of their pruritus (NRS=0 or 1) at 12 months of the study compared with 0% in the placebo group (FIG. 11).

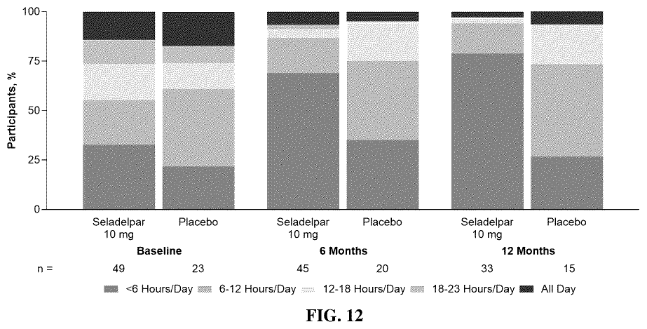

Also, reductions in 5-D Itch score over the 12-month treatment period in patients with NRS≥4 at baseline were overall greater in patients treated with seladelpar than placebo. Per 5-D Itch duration domain data, seladelpar reduced the number of hours spent itching per day as soon as 6 months and through 12 months when compared with placebo (FIG. 12).

From change from baseline in the PBC-40 total score, itch domain, and sleep disturbance question a consistent benefit on itch and sleep was seen with the PBC-40; most other domains, including fatigue, also suggested a treatment effect favoring seladelpar.

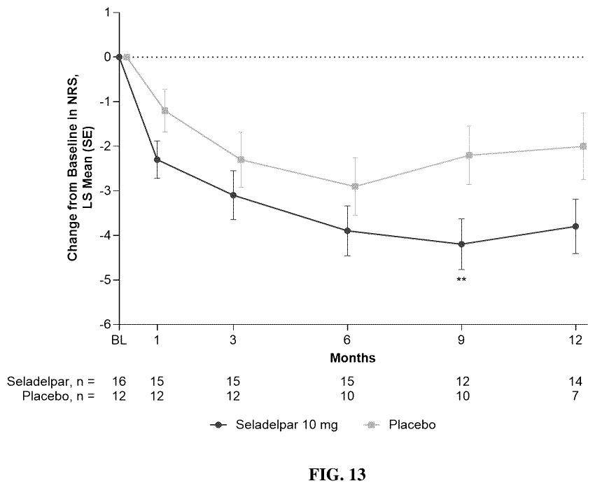

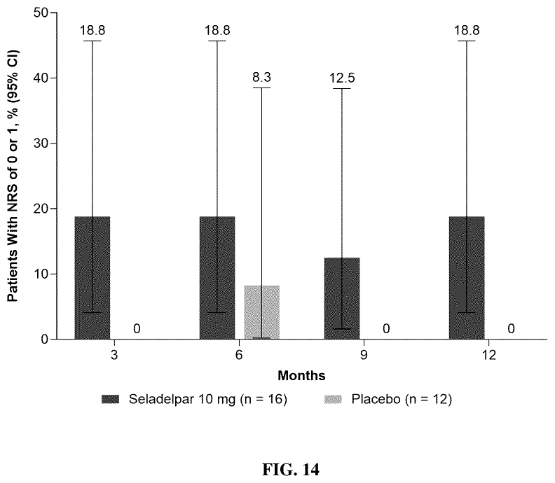

A total of 16 patients (13%) in the seladelpar group and 12 patients (18%) in the placebo group had severe pruritus (NRS≥7) at baseline. Among these patients, the reduction from baseline in NRS at 12 months was greater in patients who were treated with seladelpar than in patients who received placebo (change from baseline, −3.8 points vs −2.0 points) (FIG. 13). Further, 19% of patients who received seladelpar demonstrated near resolution of their pruritus (NRS=0 or 1) at 12 months compared with 0% in the placebo group (FIG. 14).

Reductions in 5-D Itch domains from baseline to 6 and 12 months were generally greater in patients who were treated with seladelpar than in patients who received placebo. The 5-D Itch sleep item also showed an improvement from baseline in the seladelpar group compared with the placebo group through 12 months of treatment.

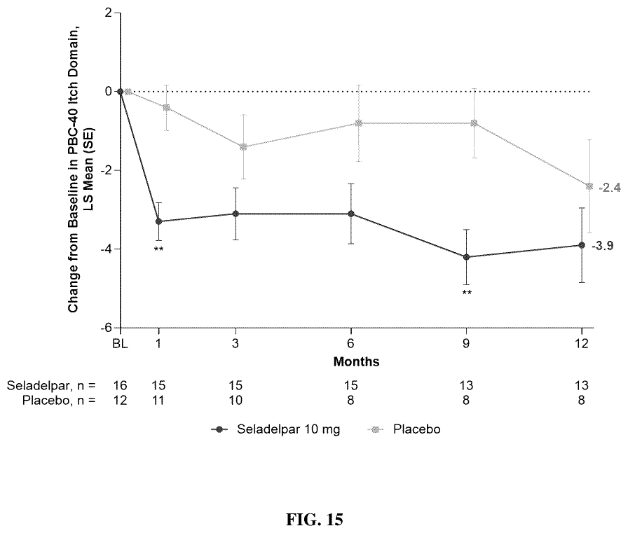

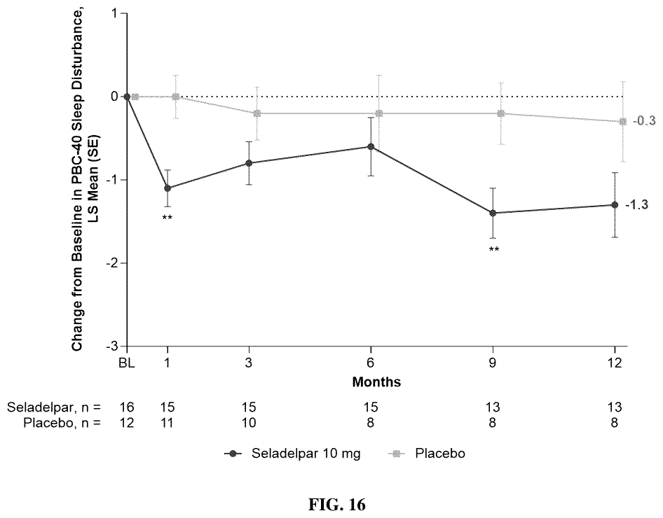

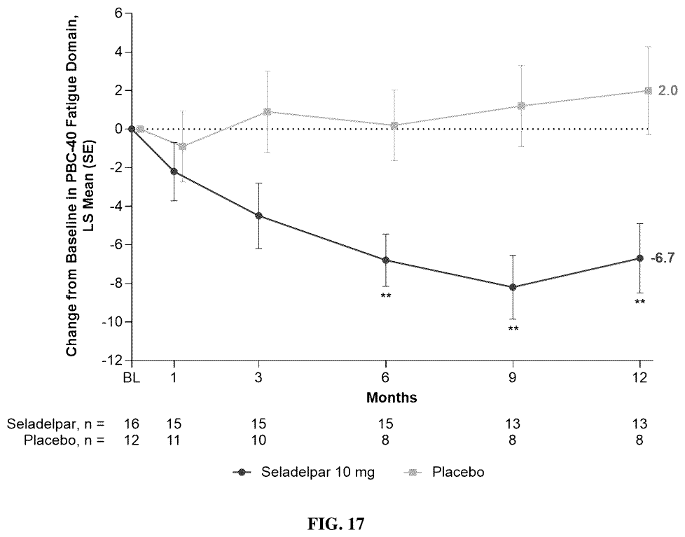

Changes from baseline in the PBC-40 itch domain, sleep disturbance question, and fatigue domain were analyzed in patients with NRS≥7 at baseline (FIGS. 15-17). A decrease in the LS mean PBC-40 itch domain score was observed as early as 1 month (P<0.05) and continued through 12 months of seladelpar treatment (FIG. 15). Improvement in the PBC-40 sleep disturbance question also occurred as soon as 1 month (P<0.05) and through 12 months of treatment (FIG. 16). Lastly, a decrease in the LS mean PBC-40 fatigue domain score was observed from 1 month through 12 months of treatment with seladelpar (FIG. 17); the LS mean difference between arms was −8.7 points at 12 months, favoring seladelpar vs placebo (P<0.05).

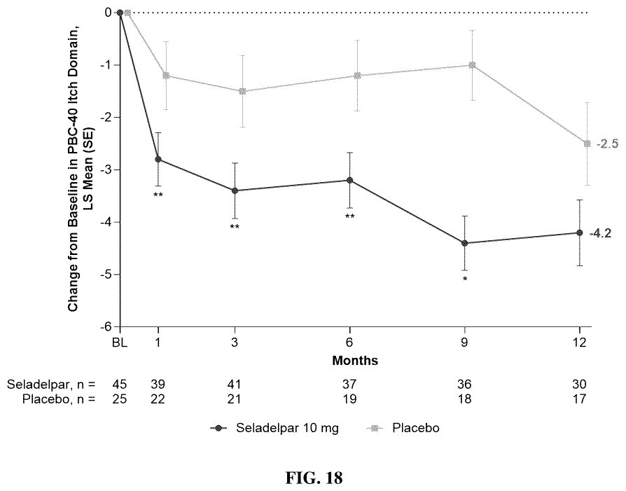

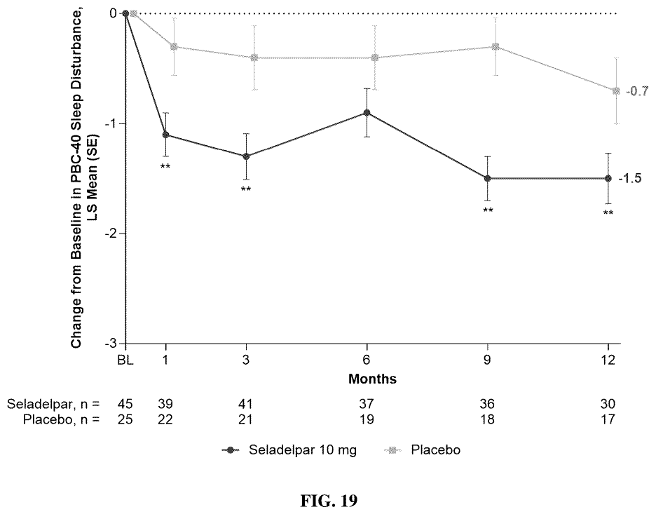

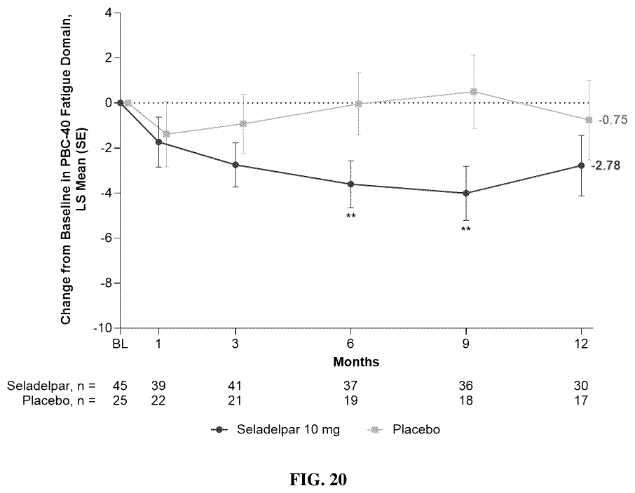

A total of 45 patients (35%) in the seladelpar group and 25 patients (38%) in the placebo group had PBC-40≥7 at baseline. Changes from baseline in the PBC-40 itch domain, sleep disturbance question, and fatigue domain were analyzed for patients with PBC-40 itch ≥7 at baseline (FIGS. 18-20). A decrease in the LS mean PBC-40 itch domain score was observed as early as 1 month (P<0.05) and continued through 12 months of seladelpar treatment (FIG. 18). Improvements from PBC-40 itch ≥7 to <7 occurred in 40% and 20% of patients receiving seladelpar and placebo, respectively, at 12 months. A decrease in the LS mean PBC-40 sleep disturbance question occurred as soon as 1 month (P<0.05) and through 12 months of treatment (FIG. 19). A decrease in the LS mean PBC-40 fatigue domain score over time also occurred in patients with PBC-40 itch 7 at baseline who were treated with seladelpar (FIG. 20).

For overall trial population, there was a pattern of improvement with shifts to more mild or moderate pruritus observed with seladelpar relative to placebo.

A responder analysis assessed patients with no itch or near no itch at baseline. At baseline, 49 patients (38%) in the seladelpar group and 25 patients (38%) in the placebo group had no or near no itch (NRS=0 or 1). Of these patients, 6% receiving seladelpar and 12% receiving placebo had an NRS score ≥2 at 12 months.

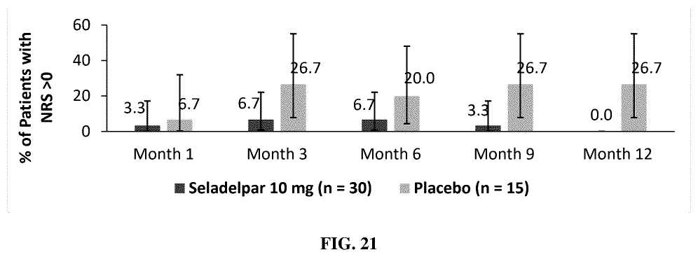

FIG. 21 shows % of patients with NRS>0 after seladelpar or placebo treatment, among patients who had no itch (NRS=0) at baseline. As shown in FIG. 21, among patients without itch at baseline, no patient receiving seladelpar developed pruritus at Month 12. Accordingly, it is contemplated that seladelpar is effective in preventing pruritus in patients who does not have pruritus before treatment.

Additionally, the proportions of patients who experienced AEs were similar in the seladelpar and placebo groups regardless of baseline itch severity (Table 6). The types and incidences of the most common AEs by preferred term were similar to those observed in the primary analysis. Pruritus reported as an AE was more common in patients receiving placebo than seladelpar.

TABLE 6

| | Patients With NRS <4 | | | | Patients With NRS ≥4 | | | |
|---|---|---|---|---|---|---|---|---|
| Patient | at Baseline | | | | at Baseline | | | |
| Incidence, n | Seladelpar | | Placebo | | Seladelpar | | Placebo | |
| (%) | (n = 79) | | (n = 42) | | (n = 49) | | (n = 23) | |
| Any AE | 68 | (86.1) | 34 | (81.0) | 43 | (87.8) | 21 | (91.3) |
| Grade ≥3 AEs | 9 | (11.4) | 2 | (4.8) | 5 | (10.2) | 3 | (13.0) |
| (per CTCAE) | | | | | | | | |
| SAEs | 5 | (6.3) | 3 | (7.1) | 4 | (8.2) | 1 | (4.3) |
| Treatment- | 0 | | 0 | | 0 | | 0 | |
| related SAEs | | | | | | | | |
| AEs leading to | 1 | (1.3) | 1 | (2.4) | 3 | (6.1) | 2 | (8.7) |
| treatment | | | | | | | | |
| discontinuation | | | | | | | | |
| AEs leading to | 0 | | 1 | (2.4) | 3 | (6.1) | 2 | (8.7) |
| study | | | | | | | | |
| discontinuation | | | | | | | | |

| | | | | |
|---|---|---|---|---|
| AEs leading to | 0 | 0 | 0 | 0 |
| death | | | | |

As shown from above, seladelpar reduced pruritus severity in patients with PBC compared with placebo, leading to clinically meaningful declines in NRS. Seladelpar reduced itch to mild levels for patients with moderate to severe pruritus, with a higher percentage of patients achieving a 3- or 4-point decline in NRS at month 12 vs placebo. Seladelpar led to near resolution of itch in almost 20% of patients and improvements in sleep and fatigue among patients with severe pruritus. Further, Seladelpar reduced itch to non-clinically significant levels in 2 times as many patients compared with placebo for patients with clinically significant itch, with improvements in quality of life, including decreases in fatigue and sleep disturbance.

On the other hand, no patient on seladelpar treatment developed pruritus compared to 27% on placebo at month 12.

Further, seladelpar was overall safe and well tolerated regardless of baseline itch.

### Example 10: Reduction in Lipids in PBC Patients with or without Statin

In the Phase 3 clinical trial of Example 9, 22% of patients in the seladelpar group (28/128) and 26% of patients in the placebo group (17/65) reported statin use at baseline. Statins are medications that can help lower cholesterol levels, which may reduce a patient's risk of heart disease and stroke.

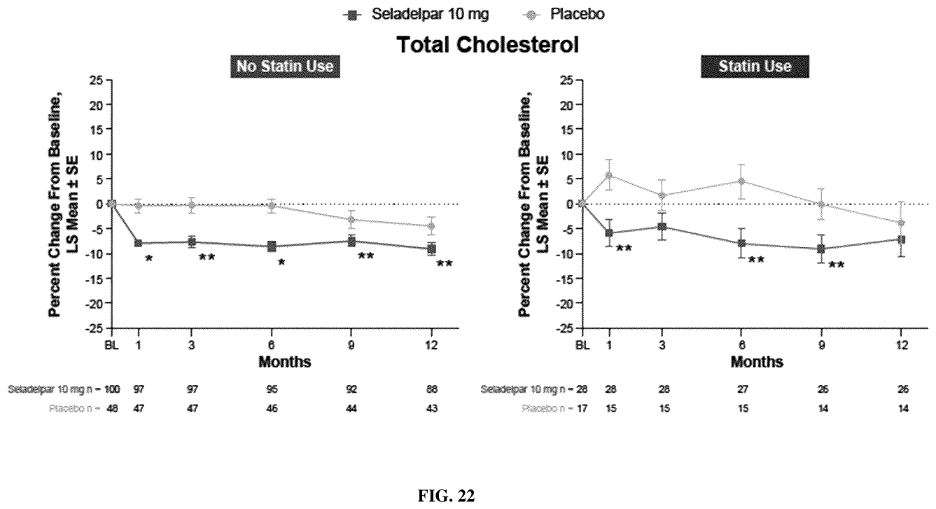

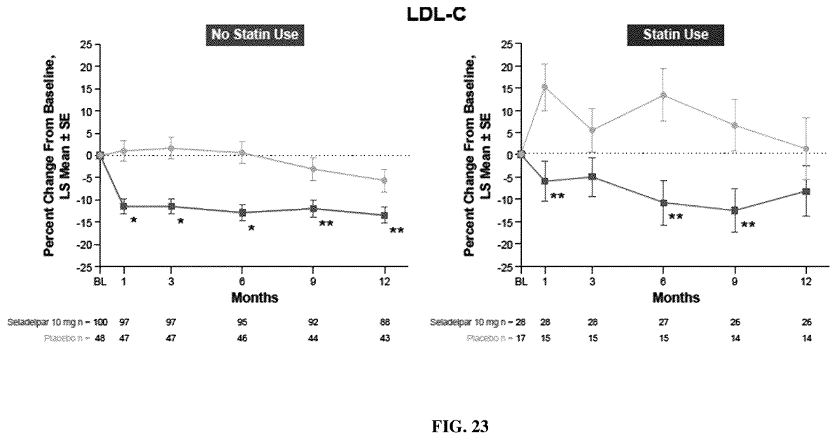

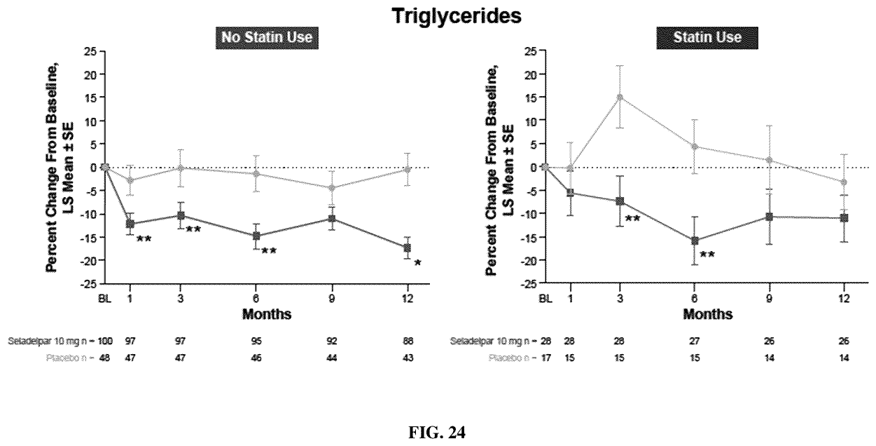

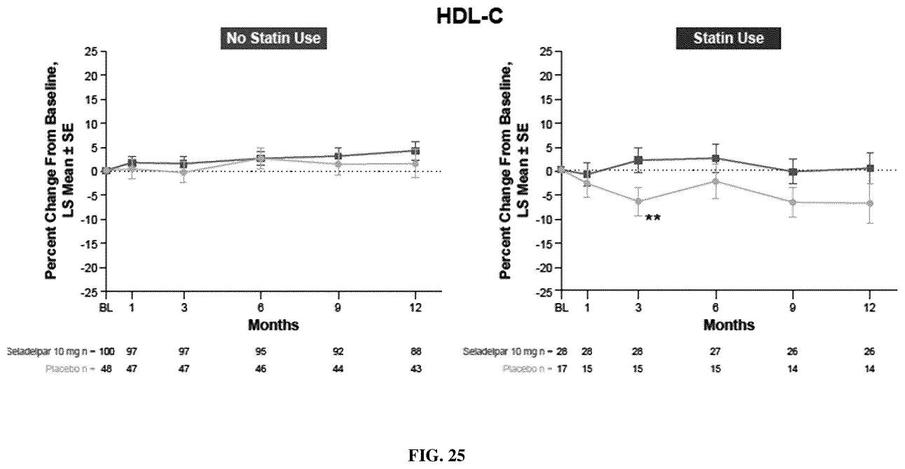

To analyze changes in lipids in patients with or without statin use at baseline among those randomized to seladelpar or placebo in the clinical trial, change from baseline in lipids was analyzed for patients receiving seladelpar or placebo by baseline statin subgroup and the results are shown in FIGS. 22-25. In FIGS. 22-25, percentage change from baseline, with or without statin, for total cholesterol level (FIG. 22), LDL level (FIG. 23), triglycerides (FIG. 24), HDL level (FIG. 25) are shown. The statin may be selected from atorvastatin, simvastatin, rospivastatin, gravistatin, and lovastatin.

As shown in FIGS. 22-25, compared with placebo, seladelpar resulted in greater reductions in total cholesterol, LDL-C, and triglyceride levels; similar results were seen between patients who were using statins at baseline and those who were not. On the other hand, in both the placebo and seladelpar treatment groups, HDL level remained stable. Accordingly, Seladelpar resulted in reductions in total cholesterol, low-density lipoprotein cholesterol, and triglyceride levels, regardless of statin use at baseline.

Further, concomitant use of seladelpar and statins was overall well tolerated, with no safety concerns identified. Incidence rates of adverse effects (AEs), serious AEs, and Grade ≥3 AEs were similar across treatment groups, regardless of statin use. Further, Muscle-related AEs also occurred at similar rates across treatment groups, regardless of statin use, and none led to treatment discontinuation. All muscle-related AEs occurring in the seladelpar group were Grades 1 or 2 in severity; one Grade 3 event of myalgia was reported in the placebo group. There were no adverse events associated with creatine kinase elevations regardless of statin use. This analysis supports that seladelpar can be administered safely to patients with PBC who are also on a statin, and suggests that lipid levels may be further improved with seladelpar use in these patients.

### Example 11: Safety of Seladelpar for Patient Subgroups

### Cirrhosis

In the Phase 3 clinical trial, approximately 14% of the population has cirrhosis. In patients with PBC and cirrhosis, seladelpar decreased cholestatic and liver injury markers compared with placebo, similar to effects seen in patients without cirrhosis in the RESPONSE trial. Patients treated with seladelpar had greater decreases in ALP, GGT, and ALT levels compared with patients receiving placebo. Total bilirubin (TB), INR, and MELD scores remained stable between the treatment groups.

The AE profile and incidence of liver enzyme elevations with seladelpar were similar to those of placebo in patients with or without compensated cirrhosis. Two patients with cirrhosis receiving seladelpar experienced liver-related AEs of hepatomegaly (Grade 1) and ascites (Grade 1), respectively; the patient with ascites then experienced an SAE of esophageal varices hemorrhage (Grade 3). All muscle-related AEs occurring in the seladelpar group were Grades 1 or 2 in severity and were not associated with creatine kinase (CK) changes. Further, there was no evidence of renal impairment in patients with or without cirrhosis. Accordingly, it is contemplated that seladelpar is safe and well tolerated in patients with PBC and compensated cirrhosis.

Cirrhosis with Portal Hypertension (PHT)

In Phase 3 clinical trials, among 56 patients who were diagnosed with cirrhosis at baseline, 27 had signs of portal hypertension (PHT) at baseline. Among them, 21 patients were randomized to seladelpar (15 of whom were on 10 mg) and 6 were randomized to placebo. These patients were analyzed for adverse effects (AEs).

Overall, 5/6 (83%) of patients on placebo and 15/21 (71%) of patients on seladelpar experienced an AE. Serious AEs (SAEs) occurred in 1/6 (17%) patients on placebo and 1/21 (5%) patients on seladelpar, but all were deemed unrelated to the study drug. Of those randomized to placebo, 2/6 (33%) patients discontinued treatment due to an AE compared with 1/21 (5%) patients randomized to seladelpar.

Incidences of liver-related AEs were similar across patients on placebo (2/6, 33%) or seladelpar (3/21, 14%); these events included hepatomegaly, ascites, hyperbilirubinaemia, and portal hypertensive gastropathy.

Liver-related laboratory abnormalities occurred in 2/6 (33%) placebo-treated patients and 1/21 (5%) seladelpar-treated patients. Of those with liver-related laboratory abnormalities, 1 patient randomized to placebo discontinued treatment, due to hyperbilirubinaemia.

Overall, in this pooled analysis of patients with primary biliary cholangitis (PBC) who had a diagnosis of cirrhosis as well as clinical signs of portal hypertension (PHT), safety outcomes and liver related adverse events (AEs) were overall similar between seladelpar and placebo. In this subpopulation of patients, treatment with seladelpar was associated with a lower postbaseline incidence of alanine aminotransferase (ALT) or aspartate aminotransferase (AST)>3×the upper limit of normal (ULN) and elevated total bilirubin (TB)>2×ULN compared with placebo. Overall, these data suggest that seladelpar can be safely administered as a second-line treatment to patients with PBC who have cirrhosis and signs of PHT.

### Example 12: Manufacturing of Seladelpar Capsule Formulation

Dosage form for seladelpar used in foregoing examples were prepared by the following process or a process similar to the following process.

Seladelpar drug substance was de-lumped (milled). Microcrystalline cellulose, de-lumped seladelpar drug substance, croscarmellose sodium, butylated hydroxytoluene, and mannitol were screened through 20-mesh screen and added to a V-blender. The screened components were blended in the V-blender for 22 minutes 30 seconds±30 seconds at 16 RPM (i.e., 360±8 revolutions). Magnesium stearate was screened through a 40-mesh screen and added to the V-blender. The components were blended in the V-blender for 5 minutes 37 seconds±30 seconds at 16 RPM (i.e., 90±8 revolutions). The pre-compaction blend (Blend 1) was discharged to double-lined PE bags.

The roller compactor was installed with “S” non-interlocking rolls and set to a target roller speed of 2 RPM, a target screw speed of 17 RPM, and a target roll force of 5,000 lbf (PAR: 4000 to 8000 lbf). Blend 1 was roller compacted at target settings and the process parameters are adjusted as required to achieve the target range ribbon density. The compacted ribbons were collected on a 20-mesh screen and manually sieved. Material passing through the 20-mesh screen was collected in double-lined PE bags as fines. Ribbons retained on 20-mesh screen were collected into double-lined PE bags. If fines were >5.0% of roller compaction output, the fines were roller compacted again (Step 2 to Step 4) and the ribbons were collected into the same double-lined PE bags in Step 5. The Comil® was set up with a 0.032″ screen (PAR: 0.024″ to 0.032″) and adjusted to 1,000±200 RPM. The ribbons (Step 5) were passed through the Comil and milled ribbons are collected into double-lined PE bags.

The quantity of extragranular components (microcrystalline cellulose, mannitol, colloidal silicon dioxide, and magnesium stearate) was adjusted based on the yield of milled ribbons (Unit Operation 3). Microcrystalline cellulose, mannitol, remaining Fines from Unit Operation 3, colloidal silicon dioxide, and milled ribbons (Step 8 from Unit Operation 3) were sieved through a 20-mesh screen and added to the V-blender. The screened extragranular components were blended in the V-blender for 9 minutes 22 seconds±30 seconds at 16 RPM (i.e., 150±8 revolutions).

Magnesium stearate was screened through a 40-mesh screen and added to the V-blender and blended for 5 minutes 37 seconds±30 seconds at 16 RPM (i.e., 90±8 revolutions) (Blend 2). Blend 2 was charged into double-lined PB bags in a HDPE drum and stored at CRT.

Resulting composition of seladelpar capsules is shown below in Table 7.

TABLE 7

| | | Conc. | Quantity (mg) per | |
|---|---|---|---|---|
| | | (% | Capsule Strength | |
| Component | Function | w/w) | 5 mg | 10 mg |
| Seladelpar drug substance¹ | Active | 8.82 | 7.052 | 14.104 |
| Mannitol | Filler | 36.93 | 29.543 | 59.086 |
| (Pearlitol ® 200 SD) | | | | |
| Microcrystalline cellulose | Diluent | 29.25 | 23.4 | 46.8 |
| (Avicel ® PH 302) | | | | |
| Microcrystalline cellulose | Diluent | 19.41 | 15.525 | 31.05 |
| (Avicel PH 101) | | | | |
| Croscarmellose sodium | Disintegrant | 3.00 | 2.4 | 4.8 |
| (Ac-di-sol ®) | | | | |
| Magnesium stearate | Lubricant | 1.50 | 1.2 | 2.4 |
| (Hyqual ®, vegetable source) | | | | |
| Colloidal silicon dioxide | Glidant | 1.00 | 0.8 | 1.6 |
| (Cab-O-Sil ® M5P) | | | | |
| Butylated hydroxytoluene | Antioxidant | 0.10 | 0.08 | 0.16 |

| | | | |
|---|---|---|---|
| Total Theoretical Capsule Fill Weight | 100 | 80.0 | 160.0 |

¹seladelpar lysine dihydrate

Unless otherwise defined, all technical and scientific terms used herein have the same meaning as commonly understood by one of ordinary skill in the art to which this invention belongs.

The inventions illustratively described herein may suitably be practiced in the absence of any element or elements, limitation, or limitations, not specifically disclosed herein. Thus, for example, the terms “comprising”, “including,” “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention has been specifically disclosed by preferred embodiments and optional features, modification, improvement, and variation of the inventions embodied therein herein disclosed may be resorted to by those skilled in the art, and that such modifications, improvements, and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

1. A method of treating primary biliary cholangitis (PBC) in a patient in need thereof, the method comprising administering a therapeutically effective amount of seladelpar or a pharmaceutically acceptable salt thereof to the patient, wherein administration of seladelpar or a pharmaceutically acceptable salt thereof results in one or more of: (a) an improvement in cholestasis; (b) an improvement in liver injury; and (c) an improvement or prevention of pruritus.

2. The method of claim 1, wherein the administration results in: (a) an improvement in cholestasis; (b) an improvement in liver injury; and (c) an improvement or prevention of pruritus.

3. The method of claim 1, wherein the administration results in one or more of: decrease and/or normalization in serum alkaline phosphatase (ALP) level, decrease and/or normalization in serum total bilirubin (TB) level, decrease in serum aspartate aminotransferase (AST) level, decrease in alanine aminotransferase (ALT) level, decrease in gamma-glutamyltransferase (GGT), decrease in pruritus Numerical Rating Scale (NRS), decrease in PBC-40 itch domain, and decrease in 5-D itch scale, wherein each decrease is relative to baseline.

4. The method of claim 1, wherein the seladelpar or a pharmaceutically acceptable salt thereof is administered for up to 12 months.

5. The method of claim 1, wherein the patient has severe pruritus (NRS≥7) at baseline.

6. The method of claim 1, wherein the patient has moderate pruritus (NRS≥4, <7) at baseline.

7. The method of claim 1, wherein the patient has moderate to severe pruritus (NRS≥4) at baseline.

8. The method of claim 1, wherein the patient has no pruritus (NRS=0) at baseline, or near no pruritus (NRS=0 or 1) at baseline.

9. The method of claim 1, wherein the patient has clinically significant pruritus (PBC-40 itch domain ≥7) at baseline.

10. The method of claim 3, wherein the patient has moderate to severe (NRS≥4) or severe (NRS>=7) pruritus at baseline, and administration of seladelpar or a pharmaceutically acceptable salt for six months or more results in complete or near resolution of pruritus (NRS=0 or 1).

11. The method of claim 4, wherein the patient has moderate to severe (NRS≥4) or severe (NRS>=7) pruritus at baseline, and administration of seladelpar or a pharmaceutically acceptable salt for about twelve months results in complete or near resolution of pruritus (NRS=0 or 1).

12. The method of claim 3, wherein the patient has moderate to severe (NRS>=4) or severe (NRS>=7) pruritus at baseline, and administration of seladelpar or a pharmaceutically acceptable salt for six months or more results in complete resolution of pruritus (NRS=0).

13. The method of claim 4, wherein the patient has moderate to severe (NRS>=4) or severe (NRS>=7) pruritus at baseline, and administration of seladelpar or a pharmaceutically acceptable salt for about twelve months results in complete resolution of pruritus (NRS=0).

14. The method of claim 1, wherein the patient has no pruritus (NRS=0) at the baseline, and does not develop pruritus (NRS=0) after administration of seladelpar or a pharmaceutically acceptable salt for about 12 months.

15. The method of claim 1, wherein the administration results in one or more of further results an improvement in at least one of sleep disturbance and fatigue.

16. The method of claim 15, wherein the administration results in decrease in decrease in at least one of PBC-40 sleep disturbance domain and fatigue.

17. The method of claim 16, wherein the patient has severe pruritus (NRS≥7) or clinically significant pruritus (PBC-40 itch domain ≥7).

18. The method of claim 1, comprising administering a therapeutically effective amount of seladelpar lysine.

19. The method of claim 1, wherein the therapeutically effective amount is equivalent to 5 mg/day or 10 mg/day of seladelpar.

20. The method of claim 1, wherein the therapeutically effective amount is equivalent to 10 mg/day of seladelpar.

21. The method of claim 1, wherein the therapeutically effective amount is equivalent to 5 mg/day of seladelpar.

22. A method of treating primary biliary cholangitis (PBC) in a patient in need thereof, the method comprising administering seladelpar or a pharmaceutically acceptable salt thereof to the patient in an amount equivalent to 5 mg/day or 10 mg/day of seladelpar, wherein administration of seladelpar or a pharmaceutically acceptable salt thereof results in one or more of: (a) an improvement in cholestasis; (b) an improvement in liver injury; and (c) an improvement or prevention of pruritus.

23. The method of claim 22, wherein the administration results in: (a) an improvement in cholestasis; (b) an improvement in liver injury; and (c) an improvement or prevention of pruritus.

24. The method of claim 22, wherein the administration results in one or more of: decrease and/or normalization in serum alkaline phosphatase (ALP) level, decrease and/or normalization in serum total bilirubin (TB) level, decrease in serum aspartate aminotransferase (AST) level, decrease in alanine aminotransferase (ALT) level, decrease in gamma-glutamyltransferase (GGT), decrease in pruritus Numerical Rating Scale (NRS), decrease in PBC-40 itch domain, and decrease in 5-D itch scale, wherein each decrease is relative to baseline.

25. The method of claim 22, wherein the seladelpar or a pharmaceutically acceptable salt thereof is administered for up to 12 months.

26. The method of claim 22, comprising administering 14.1 mg/day of seladelpar lysine dihydrate.

27. A method of treating a liver disease in a patient in need thereof, comprising administering a therapeutically effective amount of seladelpar or a pharmaceutically acceptable salt thereof to the patient, wherein the patient does not have decompensated cirrhosis.

28. The method of claim 27, wherein the liver disease is primary biliary cholangitis (PBC).

29. The method of claim 27, wherein seladelpar or a pharmaceutically acceptable salt thereof is administered orally.

30. The method of claim 27, wherein the patient is intolerant of, or has an inadequate response to ursodeoxycholic acid (UDCA) therapy.

31. The method of claim 30, wherein the patient has serum alkaline phosphatase (ALP) level at or above 1.5 times or higher of a normal level after the ursodeoxycholic acid (UDCA) therapy.

32. The method of claim 31, wherein the patient has serum alkaline phosphatase (ALP) level at or above 1.67 times or higher of a normal level after the ursodeoxycholic acid (UDCA) therapy.

33. The method of claim 27, wherein seladelpar or a pharmaceutically acceptable salt thereof is administered as a monotherapy.

34. The method of claim 27, further comprising administering a therapeutically effective amount of ursodeoxycholic acid (UDCA).

35. The method of claim 34, wherein ursodeoxycholic acid (UDCA) is administered in an amount of 5-30 mg/kg/day.

36. The method of claim 34, wherein ursodeoxycholic acid (UDCA) is administered in an amount of 13-15 mg/kg/day.

37. The method of claim 34, wherein ursodeoxycholic acid (UDCA) is administered in an amount of 18-22 mg/kg/day.

38. The method of claim 34, wherein ursodeoxycholic acid (UDCA) is administered in an amount of 8 mg/kg/day or lower.

39. The method of claim 27, comprising administering a therapeutically effective amount of seladelpar lysine.

40. The method of claim 27, wherein the amount of seladelpar is 5 mg/day or 10 mg/day.

41. The method of claim 27, further comprising monitoring for evidence of decompensation if the patient has cirrhosis.

42. A method of administering seladelpar therapy in a subject in need thereof, comprising administration of a therapeutically effective amount of seladelpar or a pharmaceutically acceptable salt thereof to a patient, wherein the patient does not have decompensated cirrhosis.

43. The method of claim 42, wherein seladelpar or a pharmaceutically acceptable salt thereof is seladelpar lysine.

44. The method of claim 42, wherein the amount of seladelpar is 5 mg/day or 10 mg/day.

45. A method of treating a liver disease in a patient in need thereof, comprising administering a therapeutically effective amount of seladelpar or a pharmaceutically acceptable salt thereof to the patient and monitoring or testing the patient for signs of drug-induced liver injury or abnormalities.

46. The method of claim 45, wherein the liver disease is primary biliary cholangitis (PBC).

47. The method of claim 45, comprising administering a therapeutically effective amount of seladelpar lysine.

48. The method of claim 45, wherein the amount of seladelpar is 5 mg/day or 10 mg/day.

49. The method of claim 45, comprising monitoring one or more of ALT, AST, total bilirubin, ALP, jaundice, fatigue, nausea, right upper quadrant pain or tenderness, fever, rash, and eosinophilia.

50. The method of claim 45, comprising monitoring for signs of muscle injury or abnormalities.

51. The method of claim 45, comprising monitoring creatine phosphokinase, muscle pain, or muscle tenderness.

52. A method of administering seladelpar therapy in a subject in need thereof, comprising administration of a therapeutically effective amount of seladelpar or a pharmaceutically acceptable salt thereof to a patient and monitoring or testing the patient for signs of drug-induced liver injury or abnormalities.

53. The method of claim 52, wherein the amount of seladelpar is 5 mg/day or 10 mg/day.

54. The method of claim 52, wherein signs of drug-induced liver injury or abnormalities comprises one or more of ALT, AST, total bilirubin, ALP, jaundice, fatigue, nausea, right upper quadrant pain or tenderness, fever, rash, and eosinophilia.

55. A method of treating a liver disease in a patient in need thereof, comprising administering a therapeutically effective amount of seladelpar or a pharmaceutically acceptable salt thereof to the patient and testing and optionally treating the patient for bone density and/or bone health.

56. The method of claim 55, wherein the liver disease is primary biliary cholangitis (PBC).

57. The method of claim 55, wherein seladelpar or a pharmaceutically acceptable salt thereof is administered orally.

58. The method of claim 55, wherein the patient is intolerant of, or has an inadequate response to ursodeoxycholic acid (UDCA).

59. The method of claim 55, wherein seladelpar or a pharmaceutically acceptable salt thereof is administered as a monotherapy.

60. The method of claim 55, further comprising administering a therapeutically effective amount of ursodeoxycholic acid (UDCA).

61. The method of claim 60, wherein ursodeoxycholic acid (UDCA) is administered in an amount of 13-15 mg/kg/day.

62. The method of claim 60, wherein ursodeoxycholic acid (UDCA) is administered at subtherapeutic dose.

63. A method of providing seladelpar therapy to a patient in need thereof, comprising administration of a therapeutically effective amount of seladelpar, further comprising monitoring and optionally treating the patient for bone density and/or bone health.

64. The method of claim 63, comprising administering a therapeutically effective amount of seladelpar lysine.

65. The method of claim 63, wherein the amount of seladelpar is 5 mg/day or 10 mg/day.

66. The method of claim 65, wherein the amount of seladelpar is 5 mg/day.

67. The method of claim 65, wherein the amount of seladelpar is 10 mg/day.

68. A method of treating a liver disease in a patient in need thereof, comprising administering a therapeutically effective amount of seladelpar or a pharmaceutically acceptable salt thereof to the patient, wherein the patient does not have complete biliary obstruction.

69. The method of claim 68, wherein the liver disease is primary biliary cholangitis (PBC).

70. The method of claim 68, wherein seladelpar or a pharmaceutically acceptable salt thereof is administered orally.

71. The method of claim 68, wherein the patient is intolerant of, or has an inadequate response to ursodeoxycholic acid (UDCA).

72. The method of claim 68, wherein seladelpar or a pharmaceutically acceptable salt thereof is administered as a monotherapy.

73. The method of claim 68, further comprising administering a therapeutically effective amount of ursodeoxycholic acid (UDCA).

74. The method of claim 68, comprising administering a therapeutically effective amount of seladelpar lysine.

75. The method of claim 68, wherein the amount of seladelpar is 5 mg/day or 10 mg/day.

76. The method of claim 68, wherein the amount of seladelpar is 5 mg/day.

77. The method of claim 68, wherein the amount of seladelpar is 10 mg/day.

78. The method of claim 68, further comprising testing the patient for biliary obstruction.

79. The method of claim 78, further comprising stopping the administration of seladelpar or a pharmaceutically acceptable salt thereof if biliary obstruction is suspected.

80. A method of providing seladelpar therapy to a patient in need thereof, comprising administering a therapeutically effective amount of seladelpar or a salt thereof, wherein the patient is not identified to have complete biliary obstruction.

81. The method of claim 80, comprising administering a therapeutically effective amount of seladelpar lysine.

82. The method of claim 80, wherein the amount of seladelpar is 5 mg/day or 10 mg/day.

83. The method of claim 82, wherein the amount of seladelpar is 10 mg/day.

84. The method of claim 80, further comprising testing the patient for biliary obstruction.

85. The method of claim 80, further comprising stopping the administration of seladelpar or a pharmaceutically acceptable salt thereof if biliary obstruction is suspected.

86. A method of treating a liver disease in a patient in need thereof, wherein the patient has renal impairment, the method comprising administering a therapeutically effective amount of seladelpar or a pharmaceutically acceptable salt thereof to the patient, wherein the amount of seladelpar is 5 mg/day or 10 mg/day.

87. The method of claim 86, wherein the liver disease is primary biliary cholangitis (PBC).

88. The method of claim 86, wherein seladelpar or a pharmaceutically acceptable salt thereof is administered orally.

89. The method of claim 86, wherein the patient is intolerant of, or has an inadequate response to ursodeoxycholic acid (UDCA).

90. The method of claim 86, wherein seladelpar or a pharmaceutically acceptable salt thereof is administered as a monotherapy.

91. The method of claim 86, further comprising administering a therapeutically effective amount of ursodeoxycholic acid (UDCA).

92. The method of claim 91, wherein UDCA is administered in an amount of 13-15 mg/kg/day.

93. The method of claim 86, comprising administering a therapeutically effective amount of seladelpar lysine.

94. The method of claim 86, wherein the renal impairment is mild, moderate, or severe renal impairment.

95. A method of administering seladelpar therapy in a patient in need thereof, wherein the patient has renal impairment, the method comprising administering a therapeutically effective amount of seladelpar or a pharmaceutically acceptable salt thereof, wherein the amount of seladelpar is 5 mg/day or 10 mg/day.

96. The method of claim 95, comprising administering a therapeutically effective amount of seladelpar lysine.

97. The method of claim 95, wherein the renal impairment is mild, moderate, or severe renal impairment.

98. A pharmaceutical composition comprising: seladelpar or a pharmaceutically acceptable salt thereof in an amount equivalent to 5 mg/day or 10 mg of seladelpar, butylated hydroxytoluene, colloidal silicon dioxide, croscarmellose sodium, magnesium stearate, mannitol, and microcrystalline cellulose.

99. The pharmaceutical composition of claim 98, comprising 14.1 mg of seladelpar lysine dihydrate.

100. The pharmaceutical composition of claim 98, wherein each of colloidal silicon dioxide, croscarmellose sodium, magnesium stearate, mannitol, and microcrystalline cellulose are substantially free of peroxides.

101. The pharmaceutical composition of claim 98, wherein the composition is substantially free of peroxides.

102. The pharmaceutical composition of claim 98, comprising granules.

103. The pharmaceutical composition of claim 98, wherein the composition is made by roller compaction and/or dry granulation.

104. A pharmaceutical dosage form comprising the pharmaceutical composition of claim 98 in a capsule.

105. A method of increasing serum level of one or more metabolism markers in a patient in need thereof, the method comprising administering a therapeutically effective amount of seladelpar or a pharmaceutically acceptable salt thereof to the patient, wherein said one or more metabolism markers are selected from the group consisting of carnitine, acetylcarnitine, acylcarnitines, N,N,N-trimethyl-5-aminovalerate, and branched-chain amino acid catabolites.

106. A method of decreasing serum level of one or more metabolism markers in a patient in need thereof, the method comprising administering a therapeutically effective amount of seladelpar or a pharmaceutically acceptable salt thereof to the patient, wherein said one or more metabolism markers are selected from dicarboxylates, saturated and unsaturated fatty acids, and lipid-derived inflammatory mediators.

107. A method of decreasing serum level of at least one cholesterol level in a patient in need thereof, the method comprising administering a therapeutically effective amount of seladelpar or a pharmaceutically acceptable salt thereof to the patient, wherein said cholesterol level is total cholesterol level, low-density lipoprotein (LDL) level, or triglyceride level.

108. The method of claim 107, wherein the method further comprises administering at least one statin to the patient, wherein the administration does not cause significant adverse effects.

109. The method of claim 107, wherein said at least one statin is selected from atorvastatin, cerivastatin, fluvastatin, lovastatin, pitavastatin, pravastatin, rosuvastatin, simvastatin, cerivastatin, analogs thereof, and a combination thereof.

110. A method of treating primary biliary cholangitis (PBC) in a patient in need thereof, the method comprising administering a therapeutically effective amount of seladelpar or a pharmaceutically acceptable salt thereof and a therapeutically effective amount of one or more statins, to the patient.

111. The method of claim 110, wherein said at least one statin is selected from atorvastatin, simvastatin, rospivastatin, gravistatin, and lovastatin.

112. The method of claim 110, wherein the therapeutically effective amount for seladelpar a pharmaceutically acceptable salt thereof or is equivalent to 5 mg/day or 10 mg/day of seladelpar.

113. The method of claim 110, wherein the administering results in reduction of at least one of total cholesterol level, low-density lipoprotein (LDL) level, and triglyceride level.

114. The method of claim 110, wherein the administering is for up to 12 months.

115. A method of increasing expression level of one or more genes selected from OCTN2, CPT1A, CPT2, CACT, and CRAT, comprising administering a therapeutically effective amount of seladelpar or a pharmaceutically acceptable salt thereof.

116. A method of determining activities of seladelpar in a subject, comprising measuring the serum level of one or more of metabolites selected from the group consisting of: carnitine, acetylcarnitine, other acylcarnitines (i.e., short-, medium- and long-chain acylcarnitines), N,N,N-trimethyl-5-aminovalerate (TMAVA), branched-chain amino acid catabolites, dicarboxylates, saturated and unsaturated fatty acids, and lipid-derived inflammatory mediators.

117. A method of determining activities of seladelpar in a subject, comprising measuring the serum level of the expression level of one or more genes selected from OCTN2, CPT1A, CPT2, CACT, and CRAT in the subject.

118. A method of treating primary biliary cholangitis (PBC) in a patient in need thereof, comprising administering a therapeutically effective amount of seladelpar or a pharmaceutically acceptable salt thereof to a subject, and measuring (a) the serum level of one or more of metabolites selected from the group consisting of: carnitine, acetylcarnitine, other acylcarnitines (i.e., short-, medium- and long-chain acylcarnitines), N,N,N-trimethyl-5-aminovalerate (TMAVA), branched-chain amino acid catabolites, dicarboxylates, saturated and unsaturated fatty acids, and lipid-derived inflammatory mediators; and/or (b) the expression level of one or more genes selected from OCTN2, CPT1A, CPT2, CACT, and CRAT in the subject.

119. The method of claim 1, wherein:
(a) the patient is less than 65 years old, or 65 years old or older;
(b) the patient was less than 50 years old at PBC diagnosis, or 50 years old or older;
(c) the patient is white, Asian, black, Latino, or non-Latino;
(d) the patient is male or female;
(e) the patient is from North America, Europe, or rest-of-world;
(f) the patient has serum ALP level of <350 U/L or ≥350 U/L at baseline;
(g) the patient has Total Bilirubin (TB) level of <0.6×ULN or ≥0.6×ULN at baseline;
(h) the patient has Total Bilirubin (TB) level of <1×ULN or ≥1×ULN at baseline;
(i) the patient has pruritus NRS of <4 or ≥4 at baseline; or
(j) the patient has cirrhosis or does not have cirrhosis.

120. A method of treating primary biliary cholangitis (PBC) in a patient in need thereof, the method comprising administering a therapeutically effective amount of seladelpar or a pharmaceutically acceptable salt thereof to the patient, wherein:
(a) the patient is less than 65 years old, or 65 years old or older;
(b) the patient was less than 50 years old at PBC diagnosis, or 50 years old or older;
(c) the patient is white, Asian, black, Latino, or non-Latino;
(d) the patient is male or female;
(e) the patient is from North America, Europe, or rest-of-world;
(f) the patient has serum ALP level of <350 U/L or ≥350 U/L at baseline;
(g) the patient has Total Bilirubin (TB) level of <0.6×ULN or ≥0.6×ULN at baseline;
(h) the patient has Total Bilirubin (TB) level of <1×ULN or ≥1×ULN at baseline;
(i) the patient has pruritus NRS of <4 or ≥4 at baseline; or
(j) the patient has cirrhosis or does not have cirrhosis.
